(19) United States
(12) Patent Application Publication
(10) Pub. No.: US 2026/0109796 A1
(43) Pub. Date: Apr. 23, 2026

(54) MATERIALS AND METHODS FOR LIGHT ADJUSTABLE OPHTHALMIC DEVICES

(71) Applicant: Johnson & Johnson Surgical Vision, Inc., Irvine, CA (US)

(72) Inventors: Shivkumar Mahadevan, Jacksonville, FL (US); Azaam Alli, Jacksonville, FL (US); Scott Joslin, Ponte Vedra, FL (US)

(21) Appl. No.: 19/360,190

(22) Filed: Oct. 16, 2025

Related U.S. Application Data
(60) Provisional application No. 63/708,680, filed on Oct. 17, 2024.

(51) Int. Cl. C08F 220/40; A61F 2/14; B29D 11/00; B29K 33/00; B29K 105/00; B29K 105/24; C07D 311/16; C07D 405/12; C08F 220/36; G02C 7/04

(52) U.S. Cl. CPC C08F 220/40; A61F 2/14; C07D 311/16; C07D 405/12; C08F 220/365; G02C 7/049; A61F 2240/001; B29D 11/00009; B29K 2033/12; B29K 2105/0002; B29K 2105/24; C08F 2800/20; C08F 2810/20

(57) ABSTRACT

This invention is directed to compounds and compositions which are designed for use in light adjustable ophthalmic devices, such as intraocular implants, intraocular lenses, phakic intraocular lenses, contact lenses, orthokeratology lenses, rigid gas permeable lenses, corneal inlays, corneal outlays, or corneal inserts. The inventive compositions are produced from reactive monomer mixtures which when polymerized form polymers or polymeric networks having editable refractive indices. Such inventive compositions when exposed to light of certain wavelengths undergo intramolecular cycloaddition reactions thereby changing the refractive index in the irradiated regions. When used in ophthalmic devices, the inventive compositions enable post-manufacturing and/or post-operative adjustments to the optics of the devices by spatially modifying the refractive index. For example, the vision of a patient with an intraocular lens made of the inventive compositions can be fine-tuned after implantation and healing by a light treatment of the intraocular lens.

### CROSS-REFERENCE TO RELATED APPLICATIONS

This application claims the benefit under 35 U.S.C. § 119(e) of U.S. Provisional Patent Application No. 63/708,680, filed Oct. 17, 2024, which is incorporated herein by reference in its entirety.

### FIELD OF THE DISCLOSURE

This invention is directed to compounds and compositions which are designed for use in light adjustable ophthalmic devices, such as intraocular implants, intraocular lenses, phakic intraocular lenses, contact lenses, orthokeratology lenses, rigid gas permeable lenses, corneal inlays, corneal outlays, or corneal inserts. The inventive compositions are produced from reactive monomer mixtures which when polymerized form polymeric networks having editable refractive indices. Such inventive compositions when exposed to light of certain wavelengths undergo intramolecular cycloaddition reactions thereby changing the refractive index in the irradiated regions. When used in ophthalmic devices, the inventive compositions enable post-manufacturing and/or post-operative adjustments to the optics of the devices by spatially modifying the refractive index. For example, the vision of a patient with an intraocular lens made of the inventive compositions can be fine-tuned after implantation and healing by a light treatment of the intraocular lens.

### BACKGROUND OF THE DISCLOSURE

Cataract surgery is commonly performed to replace the natural crystalline lens of an eye that has become opaque. Materials that are used to replace the natural crystalline lens must be soft and have excellent flexibility so that, once formed into a lens, they may be folded and passed through an incision which is typically about two millimeters. Furthermore, the material must have excellent transparency and little to no glistening. Having a high refractive index allows for a thinner lens to be used. A material with a high Abbe number demonstrates less dispersion. These physical properties, in turn, allow for improved optics and less light scattering. Combining a high refractive index with a high Abbe number provides preferable optical characteristics for an intraocular lens material.

One of the first patents in this area, U.S. Pat. No. 4,573,998, to Mazzocco, discloses a deformable intraocular lens that can be rolled to fit through a relatively small incision. The deformable lens is inserted into the eye while it is held in its rolled configuration, then released inside the chamber of the eye. The elastic properties of the lens cause it to resume its molded shape after insertion into the eye. Mazzocco discloses polyurethane elastomers, silicone elastomers, hydrogel polymer compounds, organic or synthetic gel compounds and combinations thereof as suitable materials for the deformable lens.

Friction from inside the delivery device and physician force during delivery can damage the lens. To overcome this issue, some delivery devices are coated to provide extra lubricity. For example, U.S. Pat. No. 8,323,799, to Hu, discloses a soft, flexible highly lubricious coatings for polymeric intraocular lens insertion cartridges that allow intraocular lenses to be easily inserted through small bore cartridges suitable for use with small (less than 3 millimeters) incisions. While such coatings are helpful, there is a need to further reduce the friction forces imposed on the lens during insertion, for instance, by more lubricious and tough lens materials.

Occasionally, cataract surgery leads to sub-optimal vision for the patient because of misplacement and/or misalignment of the lens during surgery and/or during the healing process requiring the use of eyeglasses or secondary surgical procedures to correct. Alternatively, lens materials have been developed in which the refractive index can be modified post-operatively by photochemical reactions, for example, by a laser beam directed to various regions of the implanted intraocular lens. In this way, the optics including the optical power of the intraocular lens can be adjusted non-invasively. Such lens materials, based on photochemically allowed, intermolecular [2+2]cycloaddition reactions of pendant coumarin groups within a polymeric network, are described in U.S. Pat. Nos. 8,109,999, 10,457,658, 10,723,713, 10,829,451, 11,001,576, 11,040,990, 11,078,177, and 11,111,226. However, these materials are limited by the mobility of the pendant coumarin groups within the polymeric network which in turn affects the rate and yield of the cycloaddition reactions responsible for modifying the optics of the implanted intraocular lens. As the cycloaddition reactions occur, the local crosslink density increases further reducing the rate of the cycloaddition reactions (by limiting the mobility of the pendant coumarin groups) as well as perhaps inducing some additional strain in the intraocular lens (crosslinking occurs between different polymer chains and if the cycloaddition reactions occur between two coumarin pendant groups on the same polymer chain, then cyclic structures are formed of various ring sizes). As a result, these materials may not have the range of adjustability needed to correct all sub-optimal conditions and may require irradiation times that are not practical.

Accordingly, there is a need for a material, with a relatively high refractive index and Abbe number, which can be used to form a flexible intraocular lens which can be simply rolled or folded into a configuration which will fit through a small incision with minimal or no damage and which can be adjusted post-operatively to improve the vision of a patient by spatially modifying the refractive index of the intraocular lens in a reasonable time and without creating potential material defects, preferably using visible light.

### SUMMARY OF THE DISCLOSURE

This invention is directed to compounds and compositions which are designed for use in light adjustable ophthalmic devices, such as intraocular implants, intraocular lenses, phakic intraocular lenses, contact lenses, orthokeratology lenses, rigid gas permeable lenses, corneal inlays, corneal outlays, or corneal inserts. The inventive compositions are produced from reactive monomer mixtures which when polymerized form polymers or polymeric networks having editable refractive indices. Such inventive compositions when exposed to light of certain wavelengths undergo intramolecular [2+2]cycloaddition reactions thereby changing the refractive index in the irradiated regions. Because the [2+2]cycloaddition reactions are intramolecular instead of intermolecular, the inventive compositions avoid the drawbacks associated with previously described technology in which pendant coumarin groups dimerize within a polymeric network, thereby forming crosslinks. When used in ophthalmic devices, the inventive compositions enable post-manufacturing and/or post-operative adjustments to the optics of the devices by spatially modifying the refractive index by selective light exposure. For example, the vision of a patient with an intraocular lens made of the inventive compositions can be fine-tuned after implantation and healing by a light treatment of the intraocular lens. Such light treatments may include spatially selective irradiation of the lens with a laser beam.

In one aspect, the invention provides for a compound having a chemical structure depicted by Formula I:

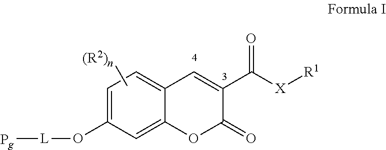

wherein P_{g} is a polymerizable group, L is a linking group, X is O or NH, R¹ is a substituent with at least one double bond capable of undergoing an intramolecular [2+2]cycloaddition reaction with the coumarin double bond in the 3-4 position to form a multi-ring structure including one cyclobutane ring, and R² is independently at each occurrence hydrogen, a C₁-C₆ alkyl group, a C₁-C₆ alkoxy group, a C₁-C₆ thioalkyl group, a C₃-C₇ cycloalkyl group, phenyl, benzyl, halo, hydroxy, amino, or NR³R⁴, wherein R³ and R⁴ are independently hydrogen or a C₁-C₆ alkyl group.

In another aspect, the invention provides for a composition made by a free radical polymerization of a reactive monomer mixture comprising a compound having a chemical structure depicted by Formula I:

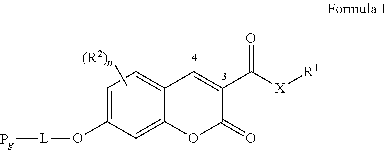

wherein P_{g} is a polymerizable group, L is a linking group, X is O or NH, R¹ is a substituent with at least one double bond capable of undergoing an intramolecular [2+2]cycloaddition reaction with the coumarin double bond in the 3-4 position to form a multi-ring structure including one cyclobutane ring, and R² is independently at each occurrence hydrogen, a C₁-C₆ alkyl group, a C₁-C₆ alkoxy group, a C₁-C₆ thioalkyl group, a C₃-C₇ cycloalkyl group, phenyl, benzyl, halo, hydroxy, amino, or NR³R⁴, wherein R³ and R⁴ are independently hydrogen or a C₁-C₆ alkyl group. In yet another aspect, the invention provides for an ophthalmic device made from any of the aforementioned compositions, such as intraocular implants, intraocular lenses, phakic intraocular lenses, contact lenses, orthokeratology lenses, rigid gas permeable lenses, corneal inlays, corneal outlays, or corneal inserts.

In more aspects, the invention provides for methods of making ophthalmic devices from the aforementioned compositions as well as for methods of modifying the refractive indices of said ophthalmic devices and thereby modifying the optical properties of said ophthalmic devices by selective irradiation using a targeting light source such as a laser.

In more aspects, the invention provides for an ophthalmic device made by a free radical polymerization of a reactive monomer mixture comprising:
a) a compound comprising a polymerizable group and at least two double bonds capable of undergoing a [2+2]cycloaddition reaction;
b) one or more monomers suitable for making the ophthalmic device;
c) a cross-linking agent;
d) a free radical initiator; and
e) one or more light absorbers selected from ultraviolet (UV) and ultraviolet/high energy visible (HEV) light absorbers, present in a total amount effective to form a protective layer that limits transmission of UV and/or HEV light beyond a penetration depth from a surface of the ophthalmic device.

### BRIEF DESCRIPTION OF FIGURES

FIG. 1 is a chart of refractive index and Abbe number as a function of irradiation time using 368 nm ultraviolet light for Example 4 discs.

FIG. 2 is a chart of refractive index and Abbe number as a function of irradiation time using 368 nm ultraviolet light for Example 5 discs.

FIG. 3 is a chart plotting the absolute value of the change in refractive index of Example 3 discs versus the fluence using 715 nm visible light.

FIG. 4 shows the UV-VIS transmission spectra of coated discs wherein the coatings absorb UV-HEV light.

FIGS. 5A-5B show infrared spectra of the exposed side and the shielded side of discs of Example 53 (FIG. 5A), and infrared spectra of a shielded side of an irradiated disc, and a non-irradiated control disc (FIG. 5B).

FIG. 6 shows infrared spectra of the exposed side and the shielded side of discs of Example 53, compared to the infrared spectrum of CECA.

FIG. 7 shows IR spectra of discs of Example 53 after 1 week of photo-exposure.

FIG. 8 shows IR spectra of discs of Example 53 after 3 weeks of photo-exposure

FIG. 9 shows FTIR spectra of exposed discs of Example 53 containing CECA (40 μm increments) after 12 weeks of photo-exposure.

FIG. 10 shows reduction in absorbance at ˜1608 and 1558 versus ˜1725 cm⁻¹ upon CECA cyclization in sample discs of Example 55 at different depths from the exposed side surface, after 8 weeks of photo-exposure.

FIG. 11 shows depth of penetration progression rates in discs of Example 54 with continuous photo-exposure for 4, 8 and 12 weeks, at 1.1 mW/cm².

FIG. 12 shows and depth of penetration progression rates in discs of Example 55 with continuous photo-exposure for 4, 8 and 12 weeks, at 1.1 mW/cm².

### DETAILED DESCRIPTION OF THE DISCLOSURE

It is to be understood that the disclosure is not limited to the details of construction or process steps set forth in the following description. The disclosure is capable of other aspects and of being practiced or being carried out in various ways using the teaching herein.

### A. Definitions

With respect to the terms used in this disclosure, the following definitions are provided.

Unless defined otherwise, all technical and scientific terms used herein have the same meaning as commonly understood by one of ordinary skill in the art to which the disclosure belongs. The polymer definitions are consistent with those disclosed in the Compendium of Polymer Terminology and Nomenclature, IUPAC Recommendations 2008, edited by: Richard G. Jones, Jaroslav Kahovec, Robert Stepto, Edward S. Wilks, Michael Hess, Tatsuki Kitayama, and W. Val Metanomski. All publications, patent applications, patents, and other references mentioned herein are incorporated by reference. High energy visible (HEV) light is described and categorized in the Spectral Bands Task Force Technical Report by the American National Standards Institute (ANSI) Accredited Standards Committee Z80. In this technical report, the terminology and nomenclature for the 380-500 nanometer spectrum of electromagnetic radiation has been standardized.

The term “reactive monomer mixture” or “reactive mixture” refers to a mixture of components (both reactive and non-reactive) which are mixed together and when subjected to polymerization conditions, form the presently disclosed compositions and ophthalmic devices. The reactive mixture may include reactive components such as monomers, macromers, prepolymers, cross-linkers, initiators, diluents, and additional components, including, but not limited to, wetting agents, release agents, dyes, light absorbing compounds, such as ultraviolet light (UV) absorbers, ultraviolet-high energy visible light (UV/HEV) absorbers, pigments, visible light (VIS) absorbers, and photochromic compounds, any of which may be reactive or non-reactive but are capable of being retained within the resulting biomedical device, e.g., an ophthalmic device, as well as active components, including pharmaceutical and nutraceutical compounds, and any diluents. It will be appreciated that a wide range of additives may be added based upon the biomedical device and its intended use. In some aspects, concentrations of components of the reactive mixture are given as weight percents of all components in the reaction mixture, excluding diluent. When diluents are used, their concentrations are given as weight percents based upon the amount of all components in the reaction mixture and the diluent.

“Reactive components” are the components in the reactive monomer mixture which become part of the chemical structure of the polymer or polymeric network of the resulting composition by covalent bonding. Diluents and processing aids which do not become part of the structure of the polymer or polymeric network are not reactive components.

“Retained components” are the polymerizable compounds (such as monomers, macromers, oligomers, prepolymers, and cross-linkers) in the reactive mixture, as well as any other components in the reactive mixture which are intended to substantially remain in the polymeric network after polymerization and all work-up steps (such as extraction steps) and packaging steps have been completed. Retained components may be retained in the polymer or polymeric network by covalent bonding, hydrogen bonding, electrostatic interactions, entanglement, the formation of interpenetrating polymeric networks, the formation of semi-interpenetrating polymeric networks, or any other means. Components that are intended to release from the biomedical device once it is in use are still considered “retained components.” For example, pharmaceutical or nutraceutical components in an ophthalmic lens which are intended to be released during use are considered “retained components.” Components that are intended to be removed from the polymer or polymeric network during the manufacturing process (e.g., by extraction), such as diluents, are “non-retained components.”

“Target macromolecule” means the macromolecule being synthesized from the reactive monomer mixture comprising monomers, macromers, prepolymers, cross-linkers, initiators, additives, diluents, and the like.

The term “polymerizable compound” means a compound containing one or more polymerizable groups. The term encompasses, for instance, monomers, macromers, oligomers, prepolymers, cross-linkers, and the like.

“Polymerizable groups” are groups that can undergo chain growth polymerization, such as free radical and/or ionic polymerization (e.g., cationic polymerization), for example, a carbon-carbon double bond which can polymerize when subjected to radical polymerization initiation conditions. Non-limiting examples of free radical polymerizable groups include (meth)acrylates, styrenes, vinyl ethers, (meth)acrylamides, N-vinyllactams, N-vinylamides, O-vinylcarbamates, O-vinylcarbonates, and other vinyl groups. Preferably, the free radical polymerizable groups comprise (meth)acrylate, (meth)acrylamide, N-vinyllactam, N-vinylamide, and styryl functional groups, and mixtures of any of the foregoing. More preferably, the free radical polymerizable groups comprise (meth)acrylates, (meth)acrylamides, and mixtures thereof. The polymerizable group may be unsubstituted or substituted. For instance, the nitrogen atom in (meth)acrylamide may be bonded to a hydrogen, or the hydrogen may be replaced with alkyl or cycloalkyl (which themselves may be further substituted).

Any type of free radical polymerization may be used including but not limited to bulk, solution, suspension, and emulsion as well as any of the controlled radical polymerization methods such as stable free radical polymerization, nitroxide-mediated living polymerization, atom transfer radical polymerization, reversible addition fragmentation chain transfer polymerization, organotellurium mediated living radical polymerization, and the like.

The term “mono-functional” refers to a component having one polymerizable group. The term “multi-functional” refers to a component having two or more polymerizable groups.

A “monomer” is a mono-functional molecule which can undergo chain growth polymerization, and in particular, free radical polymerization, thereby creating a repeating unit in the chemical structure of the target macromolecule. Some monomers have di-functional impurities that can act as cross-linking agents.

A “hydrophilic monomer” is a monomer which yields a clear single-phase solution when mixed with deionized water at 25° C. at a concentration of 5 weight percent. A “hydrophilic component” is a monomer, macromer, prepolymer, initiator, cross-linker, additive, or polymer which yields a clear single-phase solution when mixed with deionized water at 25° C. at a concentration of 5 weight percent. A “hydrophobic component” is a monomer, macromer, prepolymer, initiator, cross-linker, additive, or polymer which is slightly soluble or insoluble in deionized water at 25° C.

A “macromolecule” is an organic compound having a number average molecular weight of greater than 1500 Daltons and may be reactive or non-reactive.

A “macromonomer” or “macromer” is a macromolecule that has one group that can undergo chain growth polymerization, and in particular, free radical polymerization, thereby creating a repeating unit in the chemical structure of the target macromolecule. Typically, the chemical structure of the macromer is different than the chemical structure of the target macromolecule, that is, the repeating unit of the macromer's pendent group is different than the repeating unit of the target macromolecule or its mainchain. The difference between a monomer and a macromer is merely one of chemical structure, molecular weight, and molecular weight distribution of the pendent group. As a result, and as used herein, the patent literature occasionally defines monomers as polymerizable compounds having relatively low molecular weights of about 1,500 Daltons or less, which inherently includes some macromers. Furthermore, the patent literature occasionally defines macromers as having one or more polymerizable groups, essentially broadening the common definition of macromer to include prepolymers. As a result and as used herein, di-functional and multi-functional macromers, prepolymers, and cross-linkers may be used interchangeably.

A “polymer” is a target macromolecule composed of the repeating units of the monomers used during polymerization.

A “homopolymer” is a polymer made from one monomer; a “copolymer” is a polymer made from two or more monomers; a “terpolymer” is a polymer made from three monomers. A “block copolymer” is composed of compositionally different blocks or segments. Diblock copolymers have two blocks. Triblock copolymers have three blocks. “Comb or graft copolymers” are made from at least one macromer.

A “repeating unit” is the smallest group of atoms in a polymer that corresponds to the polymerization of a specific monomer or macromer.

An “initiator” is a molecule that can decompose into radicals which can subsequently react with a monomer to initiate a free radical polymerization reaction. A thermal initiator decomposes at a certain rate depending on the temperature; typical examples are azo compounds such as 1,1′-azobisisobutyronitrile and 4,4′-azobis(4-cyanovaleric acid), peroxides such as benzoyl peroxide, tert-butyl peroxide, tert-butyl hydroperoxide, tert-butyl peroxybenzoate, dicumyl peroxide, and lauroyl peroxide, peracids such as peracetic acid and potassium persulfate as well as various redox systems. A photo-initiator decomposes by a photochemical process; typical examples are derivatives of benzil, benzoin, acetophenone, benzophenone, camphorquinone, and mixtures thereof as well as various monoacyl and bisacyl phosphine oxides and combinations thereof.

A “cross-linking agent” is a di-functional or multi-functional monomer or macromer which can undergo free radical polymerization at two or more locations on the molecule, thereby creating branch points and a polymeric network. Common examples are ethylene glycol dimethacrylate, tetraethylene glycol dimethacrylate, trimethylolpropane trimethacrylate, methylene bisacrylamide, triallyl cyanurate, and the like.

A “prepolymer” is a reaction product of monomers which contains remaining polymerizable groups capable of undergoing further reaction to form a polymer.

As used herein, the term “(meth)” designates optional methyl substitution. Thus, a term such as “(meth)acrylates” denotes both methacrylates and acrylates.

Wherever chemical structures are given, it should be appreciated that alternatives disclosed for the substituents on the structure may be combined in any combination. Thus, if a structure contained substituents R* and R**, each of which contained three lists of potential groups, 9 combinations are disclosed. The same applies for combinations of properties.

When a subscript, such as “n” in the generic formula [***]n, is used to depict the number of repeating units in a polymer's chemical formula, the formula should be interpreted to represent the number average molecular weight of the macromolecule.

The term “individual” includes humans and non-human vertebrates.

The term “biomedical device” refers to any article that is designed to be used while either in or on mammalian tissues or fluids, and preferably in or on human tissue or fluids. Examples of these devices include but are not limited to wound dressings, sealants, tissue fillers, drug delivery systems, coatings, adhesion prevention barriers, catheters, implants, stents, and ophthalmic devices such as intraocular implants, intraocular lenses, and contact lenses. The biomedical devices may be ophthalmic devices, particularly ophthalmic implants or ophthalmic lenses made from the reactive monomer compositions described herein.

The term “ocular surface” includes the surface and glandular epithelia of the cornea, conjunctiva, lacrimal gland, accessory lacrimal glands, nasolacrimal duct and meibomian gland, and their apical and basal matrices, puncta and adjacent or related structures, including eyelids linked as a functional system by both continuity of epithelia, by innervation, and the endocrine and immune systems.

The term “ophthalmic device” refers to any device which resides in or on the eye or any part of the eye, including the ocular surface. These devices can provide optical correction, cosmetic enhancement, vision enhancement, therapeutic benefit (for example as bandages) or delivery of active components such as pharmaceutical and nutraceutical components, or a combination of any of the foregoing. Examples of ophthalmic devices include but are not limited intraocular implants, intraocular lenses, phakic intraocular lenses, contact lenses, orthokeratology lenses, rigid gas permeable lenses, corneal inlays, corneal outlays, or corneal inserts.

“Intraocular lens” refers to a lens implanted in an eye. In some aspects, the intraocular lens is implanted in the eye to replace an existing crystalline lens (such as, for example, because the existing lens has been clouded over by a cataract, or as a form of refractive surgery to change the eye's optical power).

The term “contact lens” refers to an ophthalmic device that can be placed on the cornea of an individual's eye. The contact lens may provide corrective, cosmetic, or therapeutic benefit, including wound healing, the delivery of drugs or nutraceuticals, diagnostic evaluation or monitoring, ultraviolet light absorption, visible light absorption, or glare reduction, or any combination thereof. A contact lens can be made of any appropriate material known in the art and can be a soft lens, a hard lens, or a hybrid lens containing at least two distinct portions with different physical, mechanical, or optical properties, such as modulus, water content, light transmission, or combinations thereof.

The terms “halogen” or “halo” indicate fluorine, chlorine, bromine, and iodine.

“Alkyl” or “aliphatic” are used interchangeably herein and refer to an optionally substituted linear or branched alkyl group containing the indicated number of carbon atoms and may contain double bonds, e.g., allyl. If no number is indicated, then alkyl (including any optional substituents on alkyl) may contain any of 1 to 16 carbon atoms, including 1, 2, 3, 4, 5, 6, 7, 8, 9, 10, 11, 12, 13, 14, 15, and 16 carbon atoms. Preferably, the alkyl group contains 1 to 10 carbon atoms, including 1, 2, 3, 4, 5, 6, 7, 8, 9, and 10 carbon atoms, alternatively 1 to 8 carbon atoms, including 1, 2, 3, 4, 5, 6, 7, and 8 carbon atoms, alternatively 1 to 6 carbon atoms, including 1, 2, 3, and 4 carbon atoms, or alternatively 1 to 4 carbon atoms, including 1, 2, 3, and 4. Examples of alkyl include methyl, ethyl, propyl, isopropyl, butyl, iso-, sec- and tert-butyl, pentyl, hexyl, heptyl, 3-ethylbutyl, and the like. Examples of substituents on alkyl include 1, 2, or 3 groups independently selected from hydroxy, amino, amido, oxa, carboxy, alkyl carboxy, carbonyl, alkoxy, thioalkyl, carbamate, carbonate, halogen, phenyl, benzyl, and combinations thereof. “Heteroalkyl” or “heteroaliphatic” refers to an alkyl or aliphatic group wherein at least one methylene group —CH₂— is replaced by O, S, NH, NR^{ζ} (where R^{ζ} is C₁-C₂₀ alkyl group), ketone —CO—, ester —CO₂— including carbonate —OCO₂—, amide —CONH— or —CONR^{ζ} (where R^{ζ} is C₁-C₂₀ alkyl group), urea —NHCONH— or —NR^{ζ} CONR^{ζ}— (where R^{ζ} is C₁-C₂₀ alkyl group), urethane-NHCO₂— or —NR^{ζ} CO₂— (where R^{ζ} is C₁-C₂₀ alkyl group) and combinations thereof “Alkylene” means a divalent alkyl group, such as —CH₂—, —CH₂CH₂—, —CH₂CH₂CH₂—, —CH₂CH(CH₃)CH₂—, and —CH₂CH₂CH₂CH₂—.

“Haloalkyl” refers to an alkyl group as defined above substituted with one or more halogen atoms, where each halogen is independently F, Cl, Br or I. A preferred halogen is F. Preferred haloalkyl groups contain 1-6 carbons, more preferably 1-4 carbons, and still more preferably 1-2 carbons. “Haloalkyl” includes perhaloalkyl groups, such as —CF₃— or —CF₂CF₃—. “Haloalkylene” means a divalent haloalkyl group, such as —CH₂CF₂—.

“Hydroxy” refers to an —OH group.

“Hydroxyalkyl” refers to an alkyl group, as defined herein, substituted with at least one hydroxy group. Representative examples of hydroxyalkyl include, but are not limited to, hydroxymethyl, 2-hydroxyethyl, 3-hydroxypropyl, 2,3-dihydroxypropyl, 2,3-dihydroxypentyl, 4-hydroxybutyl, 2-ethyl-4-hydroxyheptyl, 3,4-dihydroxybutyl, and 5-hydroxypentyl.

“Cycloalkyl” or “cycloaliphatic” are used interchangeably herein and refer to an optionally substituted cyclic hydrocarbon containing the indicated number of ring carbon atoms. If no number is indicated, then cycloalkyl may contain 3 to 20 ring carbon atoms (e.g., 3 to 12 ring carbon atoms). Cycloaliphatic groups can be monocyclic, bicyclic, tricyclic, bridged, fused, and/or spirocyclic. Cycloaliphatic groups can also have one or more double bonds, provided that the group is not fully aromatic. Preferred monocyclic cycloaliphatic groups are C₃-C₈ cycloalkyl groups, C₃-C₇ cycloalkyl, more preferably C₄-C₇ cycloalkyl, and still more preferably C₅-C₆ cycloalkyl. Examples of cycloalkyl include cyclopropyl, cyclobutyl, cyclopentyl, cyclohexyl, cycloheptyl and cyclooctyl. Cycloaliphatic groups also include cycloalkylalkyl groups such as cyclohexylmethyl, cyclohexylethyl, cyclohexylpropyl and the like. Examples of substituents on cycloalkyl or cycloaliphatic groups include 1, 2, or 3 groups independently selected from alkyl, hydroxy, amino, amido, oxa, carbonyl, alkoxy, thioalkyl, amido, carbamate, carbonate, halo, phenyl, benzyl, and combinations thereof. Moreover, cycloaliphatic groups can comprise cyclic ketones, cyclic esters (lactones), cyclic amides (lactams), cyclic carbonates, cyclic ureas, cyclic urethanes, and combinations thereof, any of which may further include optional substituents. “Cycloalkylene” means a divalent cycloalkyl group, such as 1,2-cyclohexylene, 1,3-cyclohexylene, or 1,4-cyclohexylene.

“Heterocycloalkyl” or “heterocycloaliphatic” refers to a cycloalkyl ring or ring system as defined above in which at least one ring carbon has been replaced with a heteroatom selected from nitrogen, oxygen, and sulfur. The heterocycloalkyl ring is optionally fused to or otherwise attached to other heterocycloalkyl rings and/or non-aromatic hydrocarbon rings and/or phenyl rings. Preferred heterocycloalkyl groups have from 5 to 7 members. More preferred heterocycloalkyl groups have 5 or 6 members. Moreover, heterocycloaliphatic groups can comprise cyclic ketones, cyclic esters (lactones), cyclic amides (lactams), cyclic carbonates, cyclic ureas, cyclic urethanes, and combinations thereof, any of which may further include optional substituents. “Heterocycloalkylene” means a divalent heterocycloalkyl group.

“Aryl” refers to an optionally substituted aromatic hydrocarbon ring system containing at least one aromatic ring. The aryl group contains the indicated number of ring carbon atoms. If no number is indicated, then aryl may contain 6 to 14 ring carbon atoms. The aromatic ring may optionally be fused or otherwise attached to other aromatic hydrocarbon rings or non-aromatic hydrocarbon rings. Examples of aryl groups include phenyl, naphthyl, and biphenyl. Preferred examples of aryl groups include phenyl. Examples of substituents on aryl include 1, 2, or 3 groups independently selected from alkyl, hydroxy, amino, amido, oxa, carboxy, alkyl carboxy, carbonyl, alkoxy, thioalkyl, carbamate, carbonate, halo, phenyl, benzyl, and combinations thereof. “Arylene” means a divalent aryl group, for example 1,2-phenylene, 1,3-phenylene, or 1,4-phenylene.

“Arylalkyl” refers to an aryl group, as defined herein, appended to the parent molecular moiety through an alkyl group, as defined herein. Representative examples of arylalkyl include phenylmethyl (i.e. benzyl), phenylethyl, and phenylpropyl.

“Heteroaryl” refers to an aryl ring or ring system, as defined above, in which at least one ring carbon atom has been replaced with a heteroatom selected from nitrogen, oxygen, and sulfur. The heteroaryl ring may be fused or otherwise attached to one or more heteroaryl rings, aromatic or nonaromatic hydrocarbon rings or heterocycloalkyl rings. Examples of heteroaryl groups include pyridyl, furyl, and thienyl. “Heteroarylene” means a divalent heteroaryl group.

“Alkoxy” refers to an alkyl group attached to the parent molecular moiety through an oxygen bridge. Examples of alkoxy groups include, for instance, methoxy, ethoxy, propoxy and isopropoxy. “Thioalkyl” means an alkyl group attached to the parent molecule through a sulfur bridge. Examples of thioalkyl groups include, for instance, methylthio, ethylthio, n-propylthio and iso-propylthio. “Aryloxy” refers to an aryl group attached to a parent molecular moiety through an oxygen bridge. Examples include phenoxy. “Arylthio” refers to an aryl group attached to a parent molecular moiety through a sulfur bridge. Examples include phenylthio. “Cyclic alkoxy” means a cycloalkyl group attached to the parent moiety through an oxygen bridge.

“Alkylamine” refers to an alkyl group attached to the parent molecular moiety through an —NH bridge. Alkyleneamine means a divalent alkylamine group, such as —CH₂CH₂NH—.

“Alkyleneoxy” refers to groups of the general formula -(alkylene-O)ₚ— or —(O-alkylene)ₚ—, wherein alkylene is as defined above, and p is from 1 to 200, or from 1 to 100, or from 1 to 50, or from 1 to 25, or from 1 to 20, or from 1 to 10, wherein each alkylene is independently optionally substituted with one or more groups independently selected from hydroxyl, halo (e.g., fluoro), amino, amido, ether, carbonyl, carboxyl, and combinations thereof. If p is greater than 1, then each alkylene may be the same or different and the alkyleneoxy may be in block or random configuration. When alkyleneoxy forms a terminal group in a molecule, the terminal end of the alkyleneoxy may, for instance, be a hydroxy or alkoxy (e.g., HO—[CH₂CH₂O]ₚ— or CH₃O—[CH₂CH₂O]ₚ—). Examples of alkyleneoxy include polyethyleneoxy, polypropyleneoxy, polybutyleneoxy, and poly(ethyleneoxy-co-propyleneoxy).

“Oxaalkylene” refers to an alkylene group as defined above where one or more non-adjacent CH₂ groups have been substituted with an oxygen atom, such as —CH₂CH₂OCH(CH₃)CH₂—. “Thiaalkylene” refers to an alkylene group as defined above where one or more non-adjacent CH₂ groups have been substituted with a sulfur atom, such as —CH₂CH₂SCH(CH₃)CH₂—.

The term “linking group” refers to a moiety that links a polymerizable group to the parent molecule. The linking group may be any moiety that is compatible with the compound of which it is a part, and that does not undesirably interfere with the polymerization of the compound, is stable under the polymerization conditions as well as the conditions for the processing and storage of the final product. For instance, the linking group may be a bond, or it may comprise one or more alkylene, haloalkylene, amide, amine, alkyleneamine, carbamate, ester (—CO₂—), arylene, heteroarylene, cycloalkylene, heterocycloalkylene, alkyleneoxy, oxaalkylene, thiaalkylene, haloalkyleneoxy (alkyleneoxy substituted with one or more halo groups, e.g., —OCF₂—, —OCF₂CF₂—, —OCF₂CH₂—), or combinations thereof. The linking group may optionally be substituted with 1 or more substituent groups. Suitable substituent groups may include those independently selected from alkyl, halo (e.g., fluoro), hydroxyl, HO-alkyleneoxy, MeO-alkyleneoxy, ether, amine, carbonyl, carbamate, and combinations thereof. The linking group may also be substituted with a polymerizable group, such as (meth)acrylate (in addition to the polymerizable group to which the linking group is linked).

Preferred linking groups include C₁-C₈ alkylene (preferably C₂-C₆ alkylene) and C₁-C₈ oxaalkylene (preferably C₂-C₆ oxaalkylene), each of which is optionally substituted with 1 or 2 hydroxy groups. Preferred linking groups also include carboxylate, amide, C₁-C₈ alkylene-carboxylate-C₁-C₈ alkylene, or C₁-C₈ alkylene-amide-C₁-C₈ alkylene.

When the linking group is comprised of combinations of moieties as described above (e.g., alkylene and cycloalkylene), the moieties may be present in any order.

The term “electron withdrawing group” (EWG) refers to a chemical group which withdraws electron density from the atom or group of atoms to which the electron withdrawing group is attached. Examples of EWGs include, but are not limited to, cyano, amide, ester, keto, or aldehyde. A preferred EWG is cyano.

The term “light absorbing compound” refers to a chemical material that absorbs light within the ultraviolet-visible spectrum (e.g., in the 100 to 780 nanometer range). A “high energy radiation absorber,” “UV/HEV absorber,” “UV/HEV absorbing compound,” or “high energy light absorbing compound” is a chemical material that absorbs various wavelengths of ultraviolet light, high energy visible light, or both. A material's ability to absorb certain wavelengths of light can be determined by measuring its UV/VIS transmission spectrum. Compounds that exhibit no absorption at a particular wavelength will exhibit substantially 100 percent transmission at that wavelength. Conversely, compounds that completely absorb at a particular wavelength will exhibit substantially 0% transmission at that wavelength. If the amount of a material's transmission is indicated as a percentage for a particular wavelength range, it is to be understood that the percentage represents the average percent transmission within that range.

When the compounds described herein contain olefinic double bonds or other centers of geometric asymmetry, and unless otherwise specified, it is intended that the compounds include the cis, trans, Z- and E-configurations. Likewise, all tautomeric and salt forms are also intended to be included.

The term “optional substituent” means that a hydrogen atom in the underlying moiety is optionally replaced by a substituent. Any substituent may be used that is sterically practical at the substitution site and is synthetically feasible. Identification of a suitable optional substituent is well within the capabilities of an ordinarily skilled artisan. Examples of an “optional substituent” include, without limitation, C₁-C₆ alkyl, C₁-C₆ alkoxy, C₁-C₆ thioalkyl, C₃-C₇ cycloalkyl, aryl, halo, hydroxy, amino, NR^{τ}R^{ττ}, benzyl, SO₃H, or SO₃Na, wherein R^{τ} and R^{ττ} are independently H or C₁-C₆ alkyl. The foregoing substituents may be optionally substituted by an optional substituent (which, unless otherwise indicated, is preferably not further substituted). For instance, alkyl may be substituted by halo (resulting, for instance, in CF₃).

Unless otherwise indicated, ratios, percentages, parts, and the like are by weight.

Unless otherwise indicated, numeric ranges, for instance as in “from 2 to 10,” are inclusive of the numbers defining the range (e.g., 2 and 10).

“Abbe number,” also known as the V-number or constringence of a transparent material, is a measure of the material's dispersion, i.e., its variation of refractive index versus wavelength, with high values indicating low dispersion. The Abbe number of a material is defined as: V_{D}=(N_{D}−1)/(N_{F}−N_{C}), where N_{D}, N_{F} and N_{C} are the refractive indices of the material at the wavelengths of the Fraunhofer D-, F- and C-spectral lines 589.3 nanometers, 486.1 nanometers and 656.3 nanometers, respectively).

“Refractive index” is defined as: n=c/v, where c is the speed of light in a vacuum and v is the phase velocity of light in the medium.

### B. Compounds

This invention is directed to compounds (referred to as “inventive compounds” herein and are the subject matter of this disclosure) which are designed for use in light adjustable ophthalmic devices, such as intraocular implants. The inventive compounds are monomers used in reactive monomer mixtures which when polymerized form polymers or polymeric networks having editable refractive indices. The inventive compounds provide pendant coumarin moieties in the polymers or polymeric networks which can undergo intramolecular [2+2]cycloaddition reactions when exposed to light of certain wavelengths, thereby changing the refractive index in the irradiated regions due to the loss of conjugation. When used in ophthalmic devices such as intraocular lenses, the optical properties, such as the power of the lens, can be adjusted post-operatively by a laser treatment.

In one aspect, the invention provides for a compound having a chemical structure depicted by Formula I:

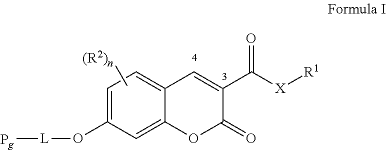

wherein P_{g} is a polymerizable group, L is a linking group, X is O or NH, R¹ is a substituent with at least one double bond capable of undergoing an intramolecular [2+2]cycloaddition reaction with the coumarin double bond in the 3-4 position to form a multi-ring structure including one cyclobutane ring, and R² is independently at each occurrence hydrogen, a C₁-C₆ alkyl group, a C₁-C₆ alkoxy group, a C₁-C₆ thioalkyl group, a C₃-C₇ cycloalkyl group, phenyl, benzyl, halo, hydroxy, amino, or NR³R⁴, wherein R³ and R⁴ are independently hydrogen or a C₁-C₆ alkyl group. In addition to the cyclobutane ring, the multi-ring structure formed by the [2+2]cycloaddition reaction can contain rings having between four and twenty-five carbon atoms; between four and fifteen carbon atoms; or between four and ten carbon atoms.

The polymerizable group in Formula I can be a (meth)acrylate, (meth)acrylamide, N-vinyl lactam, N-vinyl amide, vinyl carbonate, vinyl carbamate, vinyl ether, or styryl; preferably the polymerizable group is (meth)acrylate.

The linking group in Formula I can be an alkylene, oxaalkylene, alkyleneoxy, cycloalkylene, heterocycloalkylene, arylene, heteroarylene, alkylene-amide-alkylene, alkylene-amine-alkylene, or combinations thereof. Preferably, the linking group is an alkylene having between one and twenty-five carbon atoms; more preferably, the linking group is hexamethylene (CH₂)₆.

The R¹ group in Formula I can be any substituent with at least one double bond capable of undergoing an intramolecular [2+2]cycloaddition reaction with the coumarin double bond in the 3-4 position to form a multi-ring structure including one cyclobutane ring. In some aspects, the R¹ group can aliphatic, cycloaliphatic, aryl, heteroaliphatic, heterocycloaliphatic, heteroaryl, or combinations thereof. In other aspects, the R¹ group can be allyl, 2-cyclohexylidene-ethyl, furan-2-ylmethyl or —[CR⁵R⁶]ₙCH═CR⁷R⁸, wherein R⁵, R⁶, R⁷, and R⁸ are independently hydrogen or a C₁-C₆ alkyl group, or when R⁷ and R⁸ are connected to form cycloaliphatic ring, the cycloaliphatic ring can contain between five and eight carbon atoms and wherein n is between 1 and 20, e.g., between 1 and 10, or between 1 and 5.

Preferably, the R¹ group in Formula I has the chemical structure:

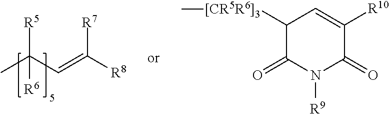

wherein R⁵, R⁶, R⁷, and R⁸ are independently hydrogen or a C₁-C₆ alkyl group, or when R⁷ and R⁸ are connected to form cycloaliphatic ring, the cycloaliphatic ring can contain between five and eight carbon atoms; and wherein R⁹ and R¹⁰ are independently hydrogen or a C₁-C₂₀ alkyl group.

Some preferred examples of the inventive compounds of Formula I are 3,7-dimethyloct-6-en-1-yl 7-((6-(acryloyloxy)hexyl)oxy)-2-oxo-2H-chromene-3-carboxylate, having the chemical structure shown in Formula II:

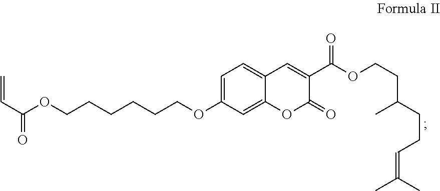 [SMILES: C=CC(=O)OCCCCCCOc1ccc2cc(C(=O)OCCC(C)CCC=C(C)C)c(=O)oc2c1]

3-(3-(2-ethylhexyl)-5-methyl-2,4-dioxo-3,4-dihydropyrimidin-1(2H)-yl)propyl 7-((6-(acryloyloxy)hexyl)oxy)-2-oxo-2H-chromene-3-carboxylate, having the chemical structure shown in Formula III:

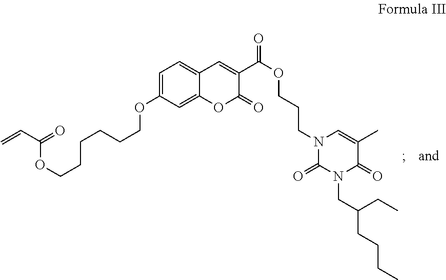 [SMILES: C=CC(=O)OCCCCCCOc1ccc2cc(C(=O)OCCCn3cc(C)c(=O)n(CC(CC)CCCC)c3=O)c(=O)oc2c1]

3-(3-butyl-5-methyl-2,4-dioxo-3,4-dihydropyrimidin-1(2H)-yl)propyl 7-((6-(acryloyloxy)hexyl)oxy)-2-oxo-2H-chromene-3-carboxylate, having the chemical structure shown in Formula IV:

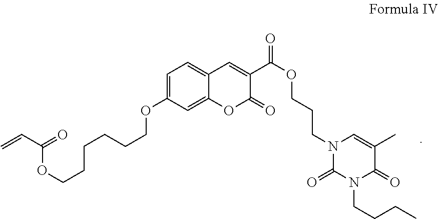 [SMILES: C=CC(=O)OCCCCCCOc1ccc2cc(C(=O)OCCCn3cc(C)c(=O)n(CCCC)c3=O)c(=O)oc2c1]

### C. Method of Making the Inventive Compounds

The inventive compounds of Formula I can be prepared by the two-step synthesis shown in Scheme 1. The first step is a piperidine-promoted, Pechmann condensation between a 2,4-dihydroxybenzaldehyde and a disubstituted malonate or malonamide, and the second step is an alkylation reaction at the 7-hydroxy position using a substrate P_{g}-L-Cl having a suitable leaving group such as but not limited to Cl as shown in Scheme 1 (see Example 1).

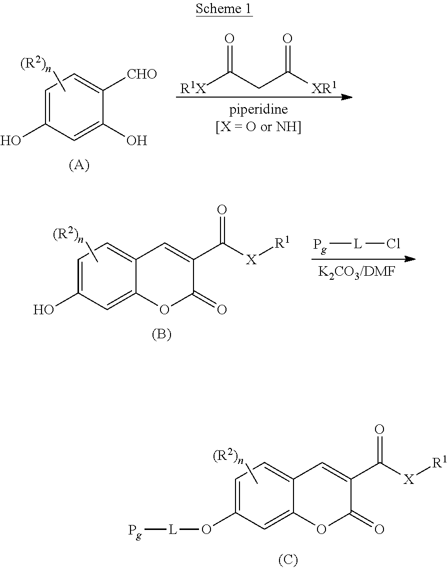

Alternatively, diethyl malonate (or equivalent) can be used to make an intermediate coumarin ethyl ester, that is, compound (B) in Scheme 1, which can then be alkylated for example using 2-((6-chlorohexyl)oxy)tetrahydro-2H-pyran at the 7-hydroxy position to form ethyl 2-oxo-7-((6-((tetrahydro-2H-pyran-2-yl)oxy)hexyl)oxy)-2H-chromene-3-carboxylate, which can then by hydrolyzed to form for example 2-oxo-7-((6-((tetrahydro-2H-pyran-2-yl)oxy)hexyl)oxy)-2H-chromene-3-carboxylic acid. Then, the protecting group can be removed, and the resulting hydroxy group acylated with a polymerizable acid chloride, for example, acryloyl chloride, to form, 7-((6-((meth)acryloyloxy)hexyl)oxy)-2-oxo-2H-chromene-3-carboxylic acid. Finally, the carboxylic acid group is converted into an ester or amide group using any known means of esterification or amide formation, such as carbodiimide coupling (see Example 2).

### D. Compositions

This invention is directed to compositions (referred to as “inventive compositions” herein and are the subject matter of this disclosure) which are designed for use in light adjustable ophthalmic devices, such as intraocular implants, intraocular lenses, phakic intraocular lenses, contact lenses, orthokeratology lenses, rigid gas permeable lenses, corneal inlays, corneal outlays, or corneal inserts. The inventive compositions are produced from reactive monomer mixtures comprising the inventive compound of Formula I which when polymerized form polymers or polymeric networks having editable refractive indices. Such inventive compositions when exposed to light of certain wavelengths undergo intramolecular cycloaddition reactions thereby changing the refractive index in the irradiated regions. When used in ophthalmic devices, such as intraocular lenses, the inventive compositions enable post-manufacturing and/or post-operative adjustments to the optics of the devices by spatially modifying the refractive index by selective light exposure. For example, the vision of a patient with an intraocular lens made of the inventive compositions can be fine-tuned after implantation and healing by a laser treatment of the intraocular lens.

In one aspect, the invention provides a composition made by free radical polymerization of a reactive monomer mixture comprising: a compound having the chemical structure depicted by Formula I:

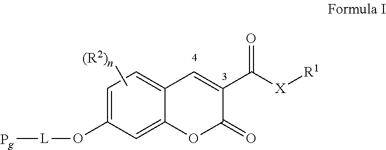

wherein P_{g} is a polymerizable group, L is a linking group, X is O or NH, R¹ is a substituent with at least one double bond capable of undergoing an intramolecular [2+2]cycloaddition reaction with the coumarin double bond in the 3-4 position to form a multi-ring structure including one cyclobutane ring, and R² is independently at each occurrence hydrogen, a C₁-C₆ alkyl group, a C₁-C₆ alkoxy group, a C₁-C₆ thioalkyl group, a C₃-C₇ cycloalkyl group, phenyl, benzyl, halo, hydroxy, amino, or NR³R⁴, wherein R³ and R⁴ are independently hydrogen or a C₁-C₆ alkyl group. In addition to the cyclobutane ring, the multi-ring structure formed by the [2+2]cycloaddition reaction can contain rings having between four and twenty-five carbon atoms; between four and fifteen carbon atoms; or between four and ten carbon atoms.

The polymerizable group in Formula I in the inventive compositions can be (meth)acrylate, (meth)acrylamide, N-vinyl lactam, N-vinyl amide, vinyl carbonate, vinyl carbamate, vinyl ether, or styryl; preferably, the polymerizable group is (meth)acrylate.

The linking group in Formula I in the inventive compositions can be an alkylene, oxaalkylene, alkyleneoxy, cycloalkylene, heterocycloalkylene, arylene, heteroarylene, alkylene-amide-alkylene, alkylene-amine-alkylene, or combinations thereof, preferably, the linking group is an alkylene having between one and twenty-five carbon atoms; more preferably, the linking groups is hexamethylene (CH₂)₆.

The R¹ group in Formula I in the inventive composition preferably has the chemical structure selected from the group consisting of:

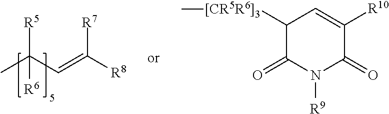

wherein R⁵, R⁶, R⁷, and R⁸ are independently hydrogen or a C₁-C₆ alkyl group, or when R⁷ and R⁸ are connected to form cycloaliphatic ring, the cycloaliphatic ring can contain between five and eight carbon atoms; and wherein R⁹ and R¹⁰ are independently hydrogen or a C₁-C₂₀ alkyl group.

Some preferred inventive compositions are made from reactive monomer mixtures including the following monomers: 3,7-dimethyloct-6-en-1-yl 7-((6-(acryloyloxy)hexyl)oxy)-2-oxo-2H-chromene-3-carboxylate, having the chemical structure shown in Formula II:

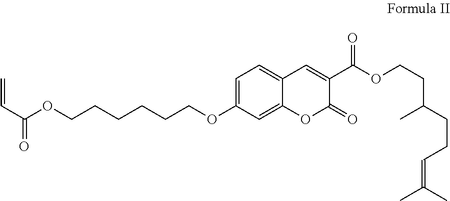 [SMILES: C=CC(=O)OCCCCCCOc1ccc2cc(C(=O)OCCC(C)CCC=C(C)C)c(=O)oc2c1]

3-(3-(2-ethylhexyl)-5-methyl-2,4-dioxo-3,4-dihydropyrimidin-1(2H)-yl)propyl 7-((6-(acryloyloxy)hexyl)oxy)-2-oxo-2H-chromene-3-carboxylate, having the chemical structure shown in Formula III:

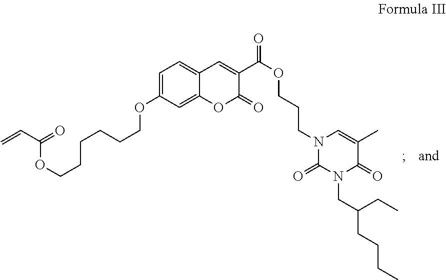 [SMILES: C=CC(=O)OCCCCCCOc1ccc2cc(C(=O)OCCCn3cc(C)c(=O)n(CC(CC)CCCC)c3=O)c(=O)oc2c1]

and 3-(3-butyl-5-methyl-2,4-dioxo-3,4-dihydropyrimidin-1(2H)-yl)propyl 7-((6-(acryloyloxy)hexyl)oxy)-2-oxo-2H-chromene-3-carboxylate, having the chemical structure shown in Formula IV:

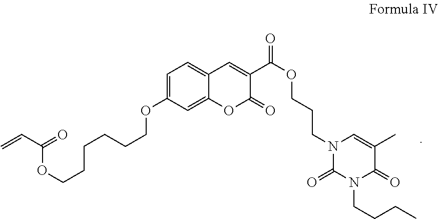 [SMILES: C=CC(=O)OCCCCCCOc1ccc2cc(C(=O)OCCCn3cc(C)c(=O)n(CCCC)c3=O)c(=O)oc2c1]

In some aspects, the inventive compositions can be made from reactive monomer mixtures having between 1 weight percent and 99 weight percent of the compound of Formula I; between 20 weight percent and 80 weight percent of the compound of Formula I; between 30 weight percent and 60 weight percent of the compound of Formula I; or between 30 weight percent and 50 weight percent of the compound of Formula I (calculated excluding diluents).

In some aspects, the inventive compositions can be made from reactive monomer mixtures further comprising additional monomers suitable for making a biomedical or ophthalmic device. The additional monomers are preferably selected from the group consisting of aliphatic (meth)acrylates, cycloaliphatic (meth)acrylates, aryl (meth)acrylates, hydrophilic monomers, and combinations thereof. The inventive compositions may further comprise cross-linking agents, polymerization initiators, light absorbing compounds, such as ultraviolet light (UV) absorbers, ultraviolet-high energy visible light (UV/HEV) absorbers, visible light (VIS) absorbers, and photochromic compounds, and diluents.

### Aliphatic (Meth)Acrylates

The aliphatic (meth)acrylates that can be used to make the inventive compositions comprise linear and branched alkyl groups having between one and twenty-five carbon atoms; preferably, linear and branched alkyl groups having between one and fifteen carbon atoms; and more preferably, linear and branched alkyl groups having between one and ten carbon atoms. In particular aspects, the aliphatic (meth)acrylates are selected from the group consisting of methyl (meth)acrylate, ethyl (meth)acrylate, n-butyl (meth)acrylate, iso-butyl (meth)acrylate, t-butyl (meth)acrylate, n-hexyl (meth)acrylate, 2-ethylhexyl (meth)acrylate, octyl (meth)acrylate, iso-decyl (meth)acrylate, heptadecyl (meth)acrylate, dodecyl (meth)acrylate, 2-propylheptyl (meth)acrylate, stearyl (meth)acrylate, and any combination thereof. In other aspects, the aliphatic (meth)acrylate comprises a linear alkyl group containing between four and eight carbon atoms. Preferred aliphatic (meth)acrylates are n-butyl (meth)acrylate and n-hexyl (meth)acrylate.

In some aspects, the aliphatic (meth)acrylate has an alkyl group with at least one carbon-carbon double bond, for example, allyl (meth)acrylate.

The aliphatic (meth)acrylates that can be used to make the inventive compositions comprise linear and branched hydroxyalkyl groups having between one and twenty-five carbon atoms; preferably, linear and branched hydroxyalkyl groups having between one and fifteen carbon atoms; and more preferably, linear and branched hydroxyalkyl groups having between one and ten carbon atoms. In particular aspects, the aliphatic (meth)acrylates are selected from the group consisting of 2-hydroxyethyl (meth)acrylate, 2-hydroxypropyl (meth)acrylate, 3-hydroxypropyl (meth)acrylate, 2,3-dihydroxypropyl (meth)acrylate, 4-hydroxybutyl (meth)acrylate, 6-hydroxyhexyl (meth)acrylate, 1,1-dimethyl-2-hydroxyethyl (meth)acrylate, and any combination thereof. In other aspects, the aliphatic (meth)acrylate comprises a linear hydroxyalkyl group containing between four and eight carbon atoms. Preferred aliphatic (meth)acrylates are 4-hydroxybutyl (meth)acrylate and 6-hydroxyhexyl (meth)acrylate.

The aliphatic (meth)acrylates that can be used to make the inventive compositions comprise linear and branched (meth)acryloxy)alkyl alkanoates having a generic chemical structure R′CO₂[CH₂]ₙOCOC═CHR″, wherein R′ is a linear or branched alkyl group, R″ is hydrogen or a methyl group, and the number of carbon atoms in the molecules is between six and thirty carbon atoms; preferably, between ten and twenty-five carbon atoms; and more preferably, between ten and twenty carbon atoms. In particular aspects, the aliphatic (meth)acrylates are selected from the group consisting of 4-((meth)acryloyloxy)butyl 2,2-dimethylbutanoate and 2-((meth)acryloyloxy)ethyl 2,2-dimethylbutanoate.

In some aspects, the reactive monomer mixtures from which the inventive compositions are made can include at least one aliphatic (meth)acrylate in an amount equal to or less than 5 weight percent; in an amount equal to or less than 10 weight percent; equal to or less than 20 weight percent; equal to or less than 30 weight percent; equal to or less than 40 weight percent; equal to or less than 50 weight percent; equal to or less than 60 weight percent; equal to or less than 70 weight percent; equal to or less than 80 weight percent; equal to or less than 90 weight percent; or equal to or less than 95 weight percent (excluding any diluents). In some aspects, the reactive monomer mixtures from which the inventive compositions are made can include at least one aliphatic (meth)acrylate between 30 weight percent and 70 weight percent; between 35 weight percent and 65 weight percent; or between 40 weight percent and 60 weight percent (calculated excluding any diluents).

### Cycloaliphatic (Meth)Acrylates

The cycloaliphatic (meth)acrylates that can be used to make the inventive compositions comprise cycloaliphatic groups having between one and four cycloaliphatic rings, and the rings can be monocyclic, bicyclic, tricyclic, bridged, fused, and/or spirocyclic. The cycloaliphatic (meth)acrylates can also have one or more double bonds in the rings, provided that any double bond conjugation is not fully aromatic.

In some aspects, the cycloaliphatic (meth)acrylates are selected from cyclohexyl (meth)acrylate, cyclopentyl (meth)acrylate, cyclohexylmethyl (meth)acrylate, 2-cyclohexylethyl (meth)acrylate, 3-cyclohexylpropyl (meth)acrylate, norbornyl (meth)acrylate, isobornyl (meth)acrylate, ((1R,2S,4R)-bicyclo[2.2.1]hept-5-en-2-yl)methyl (meth)acrylate, ethylene glycol dicyclopentenyl ether (meth)acrylate, poly(ethylene glycol) dicyclopentenyl ether (meth)acrylate, 2,2-bis(cyclopent-1-en-1-yloxy)ethyl (meth)acrylate, (1R,3S,5f,7r)-2-methyladamantan-2-yl (meth)acrylate, and any combination thereof. In other aspects, the cycloaliphatic (meth)acrylate is selected from cyclohexyl (meth)acrylate, cyclopentyl (meth)acrylate, cyclohexylmethyl (meth)acrylate, 2-cyclohexylethyl (meth)acrylate, 3-cyclohexylpropyl (meth)acrylate, ethylene glycol dicyclopentenyl ether (meth)acrylate, and any combination thereof. In some aspects, the cycloaliphatic (meth)acrylate is cyclohexyl (meth)acrylate. In some aspects, the cycloaliphatic (meth)acrylate is cyclohexylmethyl (meth)acrylate. In some aspects, the cycloaliphatic (meth)acrylate is 2-cyclohexylethyl (meth)acrylate. In some aspects, the cycloaliphatic (meth)acrylate is 3-cyclohexylepropyl (meth)acrylate In some aspects, the cycloaliphatic (meth)acrylate is ethylene glycol dicyclopentenyl ether acrylate.

In some aspects, the cycloaliphatic (meth)acrylates do not include optional substituents on the cycloaliphatic ring, or ring system, or anywhere else on the monomer. In other aspects, the cycloaliphatic ring or ring system includes at least one hydroxy group.

In some aspects, the reactive monomer mixtures from which the inventive compositions are made can include at least one cycloaliphatic (meth)acrylate in an amount equal to or less than 5 weight percent; in an amount equal to or less than 10 weight percent; equal to or less than 20 weight percent; equal to or less than 30 weight percent; equal to or less than 40 weight percent; equal to or less than 50 weight percent; equal to or less than 60 weight percent; equal to or less than 70 weight percent; equal to or less than 80 weight percent; equal to or less than 90 weight percent; or equal to or less than 95 weight percent (excluding any diluents). In some aspects, the reactive monomer mixtures from which the inventive compositions are made can include at least one cycloaliphatic (meth)acrylate between 30 weight percent and 70 weight percent; between 35 weight percent and 65 weight percent; or between 40 weight percent and 60 weight percent (calculated excluding any diluents).

### Aromatic (Meth)Acrylates

The aromatic (meth)acrylates that can be used to make the inventive compositions comprise at least one aryl group. In some aspects, the aryl group is a phenyl group. In other aspects, the aryl group is an arylalkyl group (e.g., benzyl, 2-phenylethyl, 3-phenylpropyl, or 4-phenylbutyl), an aryloxyalkyl group (e.g., phenoxymethyl, 2-phenoxyethyl, or 3-phenoxypropyl), or an arylthioalkyl group (e.g., phenylthiomethyl, 2-phenylthioethyl, or 3-phenylthiopropyl). In some aspects, the aromatic (meth)acrylate is selected from 2-phenylethyl (meth)acrylate, 2-phenoxyethyl (meth)acrylate, 3-phenylpropyl (meth)acrylate, 4-phenylbutyl (meth)acrylate, 3-phenoxypropyl (meth)acrylate, 1,3-bis(phenylthio)-2-propyl (meth)acrylate, poly(ethylene glycol) phenyl ether (meth)acrylate, and any combination thereof. In other aspects, the aromatic (meth)acrylate is a combination of 2-phenylethyl acrylate and 2-phenylethyl methacrylate. The aromatic (meth)acrylate can comprise at least one aryl group with at least one optional substituent. In yet another aspect, the optional substituent is at least one aliphatic group comprising at least one carbon-carbon double bond, provided that any conjugation is not fully aromatic. In some aspects, the aromatic (meth)acrylate is cinnamyl (meth)acrylate.

In some aspects, the reactive monomer mixtures from which the inventive compositions are made can include at least one aromatic (meth)acrylate in an amount equal to or less than 5 weight percent; in an amount equal to or less than 10 weight percent; equal to or less than 20 weight percent; equal to or less than 30 weight percent; equal to or less than 40 weight percent; equal to or less than 50 weight percent; equal to or less than 60 weight percent; equal to or less than 70 weight percent; equal to or less than 80 weight percent; equal to or less than 90 weight percent; or equal to or less than 95 weight percent (excluding any diluents). In some aspects, the reactive monomer mixtures from which the inventive compositions are made can include at least one aromatic (meth)acrylate between 30 weight percent and 70 weight percent; between 35 weight percent and 65 weight percent; or between 40 weight percent and 60 weight percent (calculated excluding any diluents).

### Hydrophilic Monomers

Any hydrophilic monomer can be used to make the inventive compositions usually to balance the biological, physical, and mechanical properties of said inventive compositions. In some aspects, the hydrophilic monomer is selected from N-vinyl pyrrolidone, N-vinyl-N-methyl acetamide, N-methyl methacrylamide, N-vinyl acetamide, N,N-dimethyl acrylamide, N-hydroxyethylacrylamide, N-(2-hydroxypropyl)acrylamide, N-(3-hydroxypropyl)acrylamide, N-(2-hydroxyethyl)(meth)acrylamide, N-(2-hydroxypropyl)(meth)acrylamide, N-(3-hydroxypropyl)(meth)acrylamide, poly(ethylene glycol) methyl ether (meth)acrylate, poly(ethylene glycol) (meth)acrylate, and any combination thereof.

In some aspects, the reactive monomer mixtures from which the inventive compositions are made can include at least one hydrophilic monomer in an amount equal to or less than 5 weight percent; equal to or less than 10 weight percent; equal to or less than 20 weight percent; equal to or less than 30 weight percent; equal to or less than 40 weight percent; equal to or less than 50 weight percent; equal to or less than 60 weight percent; equal to or less than 70 weight percent; equal to or less than 80 weight percent; equal to or less than 90 weight percent; or equal to or less than 95 weight percent (excluding any diluents). In some aspects, the reactive monomer mixtures from which the inventive compositions are made can include at least one hydrophilic monomer between 30 weight percent and 70 weight percent; between 35 weight percent and 65 weight percent; or between 40 weight percent and 60 weight percent (calculated excluding any diluents).

### Cross-Linking Agents

Any cross-linking agent can be used to make the inventive compositions and polymeric networks. In some aspects, the cross-linking agent is selected from the group consisting of aliphatic cross-linking agents, cycloaliphatic cross-linking agents, aromatic cross-linking agents, and combinations thereof. Preferably, the cross-linking agents is an aliphatic cross-linking agent. In one aspect, the aliphatic cross-linking agent has between one and twenty-five carbon atoms. In another aspect, the aliphatic cross-linking agent can be oligomeric. In other aspects, the aliphatic cross-linking agent is selected from the group consisting of ethylene glycol di(meth)acrylate, diethylene glycol di(meth)acrylate, triethylene glycol di(meth)acrylate, tetraethylene glycol di(meth)acrylate, trimethylolpropane tri(meth)acrylate, glycerol tri(meth)acrylate, methylene bis(meth)acrylamide, poly(ethylene glycol) di(meth)acrylate, and any combination thereof. In some aspects, the aliphatic cross-linking agent is selected from the group consisting of ethylene glycol di(meth)acrylate, diethylene glycol di(meth)acrylate, triethylene glycol di(meth)acrylate, tetraethylene glycol di(meth)acrylate, trimethylolpropane tri(meth)acrylate, and any combination thereof.

The cycloaliphatic cross-linking agent comprises at least one cycloaliphatic group. Preferred cycloaliphatic cross-linking agents have between one and four cycloaliphatic rings. In particular aspects, the cycloaliphatic cross-linking agent is tricyclo[5.2.1.0^{2,6}]decanedimethanol di(meth)acrylate. A preferred cycloaliphatic cross-linking agent is tricyclo[5.2.1.0^{2,6}]decanedimethanol diacrylate. The aromatic cross-linking agent comprises at least one aryl group. Preferred aromatic cross-linking agents are triallyl cyanurate, 1,4-phenylene diacrylate, 1,4-phenylene dimethacrylate, 2,2-bis(4-methacryloxyphenyl)-propane, 2,2-bis[4-(2-acryloxyethoxy)phenyl]propane, 2,2-bis[4-(2-hydroxy-3-methacryloxypropoxy)-phenyl]propane, 4-vinylbenzyl methacrylate, 2,2-bis(3-allyl-4-hydroxyphenyl)propane, 2,2-bis(3-allyl-4-acetoxyphenyl)propane, divinylbenzene, and combinations thereof.

In some aspects, the reactive monomer mixtures from which the inventive compositions are made can include at least one cross-linking agent in an amount equal to or less than 2 weight percent; equal to or less than 3 weight percent; equal to or less than 4 weight percent; equal to or less than 5 weight percent; or equal to or less than 10 weight percent (excluding any diluents). In some aspects, the reactive monomer mixtures from which the inventive compositions are made can include at least one cross-linking agent between 0.1 weight percent and 10 weight percent; between 1 weight percent and 5 weight percent; or between 1 weight percent and 3 weight percent (calculated excluding any diluents). Preferred inventive compositions contain cross-linking agents and are polymeric networks.

### Initiators

Any initiator or initiator system can be used to make the inventive compositions, such as polymers and polymeric networks. In some aspects, the free radical initiator is selected from the group consisting of a thermal initiator, a photochemical initiator, and combinations thereof. The thermal initiator can be a peroxide (e.g., lauroyl peroxide and benzoyl peroxide), a hydroperoxide (e.g., cumene or t-butyl hydroperoxide), a peracid or perester (e.g., iso-propyl percarbonate), an azo compound (e.g., azobisisobutyronitrile), and the like, that generate free radicals at moderately elevated temperatures. Preferred thermal initiators are benzoyl peroxide and azobisisobutyronitrile. The photochemical initiator or photoinitiator can be various derivatives of benzil, benzoin, acetophenone, benzophenone, camphorquinone, and mixtures thereof as well as various monoacyl and bisacyl phosphine oxides and combinations thereof. Preferred photochemical initiators are 1-hydroxycyclohexyl phenyl ketone, 2-hydroxy-2-methyl-1-phenyl-propan-1-one, bis(2,6-dimethoxybenzoyl)-2,4-4-trimethylpentyl phosphine oxide, bis(2,4,6-trimethylbenzoyl)-phenyl phosphine oxide, 2,4,6-trimethylbenzyldiphenyl phosphine oxide, benzoin methyl ester and a combination of camphorquinone and ethyl 4-(N,N-dimethylamino)benzoate. A more preferred photochemical initiator is bis(2,4,6-trimethylbenzoyl)-phenyl phosphineoxide.

Commercially available (from IGM Resins B.V., The Netherlands) visible light initiator systems include Omnirad 819, Omnirad 1700, Omnirad 1800, Omnirad 819, Omnirad 1850 and Lucrin® TPO. Commercially available (from IGM Resins B.V.) UV photoinitiators include Darocur® 1173 and Darocur® 2959. These examples and other photoinitiators which can be used are disclosed in Volume III, Photoinitiators for Free Radical Cationic & Anionic Photopolymerization, 2nd Edition by J. V. Crivello & K. Dietliker; edited by G. Bradley; John Wiley and Sons; New York; 1998.

In some aspects, the reactive monomer mixtures from which the inventive compositions are made can include at least one free radical initiator between 0.01 weight percent and 5 weight percent; between 0.1 weight percent and 1 weight percent; or between 0.1 weight percent and 0.5 weight percent (calculated excluding any diluents).

### Light Absorbing Compounds

Any light absorbing compound can be used to make the inventive compositions. The light absorbing compound can be an ultraviolet light (UV) absorber, an ultraviolet-high energy visible light (UV/IEV) absorber, visible light (VIS) absorber, and combinations thereof. Common ultraviolet and visible light absorbers are benzotriazoles, azo-dyes, acetophenones, benzophenones, and anthraquinones. Benzotriazoles are a preferred category of UV absorbers and/or UV/HEV absorbers, for example, 2-(2′-hydroxy-5-methacrylyloxyethylphenyl)-2H-benzotriazole, 3-(3-(tert-butyl)-5-(5-chloro-2H-benzo[d][1,2,3]triazol-2-yl)-4-hydroxyphenyl)propyl methacrylate, or 2-(tert-butyl)-6-(5-chloro-2H-benzo[d][1,2,3]triazol-2-yl)-4-vinylphenol.

Some preferred UV/HEV light absorbers for ophthalmic devices are disclosed in U.S. Pat. No. 10,935,695 and 11,95,824 and in U.S. Published Application No. 2020/040732 which are hereby incorporated in their entireties into this application. In one aspect, the ultraviolet-high energy visible light absorber has the chemical structure depicted by Formula V:

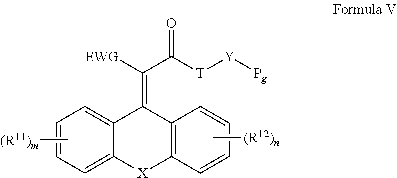

wherein m and n are independently 0, 1, 2, 3, or 4; T is a bond, O, or NR¹³; X is O, S, NR, SO, or SO₂; Y is a linking group; P_{g} is a polymerizable group; R¹³ at each occurrence is independently H, C₁-C₆ alkyl, cycloalkyl, heterocycloalkyl, aryl, heteroaryl, or Y—P_{g}; R¹¹ and R¹², when present, are independently at each occurrence C₁-C₆ alkyl, C₁-C₆ alkoxy, C₁-C₆ thioalkyl, C₃-C₇ cycloalkyl, aryl (preferably unsubstituted phenyl or phenyl substituted with alkyl or halo), halo, hydroxy, amino, NR¹⁴R¹⁵, or benzyl, wherein R¹⁴ and R¹⁵ are independently H or C₁-C₆ alkyl, or two adjacent R¹ or R¹² groups, together with the carbon atoms to which they are attached, combine to form a cycloalkyl or aryl ring; and EWG is an electron withdrawing group. A preferred EWG is cyano. In some aspects, the ultraviolet-high energy visible light absorber of Formula V is selected from the group consisting of 2-(2-cyano-2-(9H-thioxanthen-9-ylidene)acetamido)ethyl methacrylate, 2-(2-cyano-2-(9H-xanthen-9-ylidene)acetamido)ethyl methacrylate, 2-(2-cyano-2-(10-methylacridin-9(10H)-ylidene)acetamido)ethyl methacrylate, 2-(2-cyano-2-(2-methoxy-10-propylacridin-9(10H)-ylidene)acetamido)ethyl methacrylate, 2-(2-cyano-2-(2-methoxy-10-butylacridin-9(10H)-ylidene)acetamido)ethyl methacrylate, 2-(2-cyano-2-(9H-xanthen-9-ylidene)acetamido)ethyl methacrylate, (E)-2-(2-cyano-2-(3-methoxy-9H-xanthen-9-ylidene)acetamido)ethyl methacrylate, (E)-2-(2-(3-butoxy-9H-xanthen-9-ylidene)-2-cyanoacetamido)ethyl methacrylate, and any combination thereof. A preferred example of the ultraviolet-high energy visible light absorber of Formula V is 2-(2-cyano-2-(9H-thioxanthen-9-ylidene)acetamido)ethyl methacrylate.

In another aspects, the ultraviolet-high energy visible light absorber has the chemical structure depicted by Formula VI:

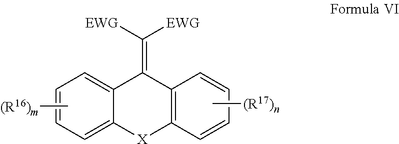

wherein m and n are independently 0, 1, 2, 3, or 4; X is O, S, NR¹⁸, SO, or SO₂; R¹⁸ at each occurrence is independently H, C₁-C₆ alkyl, cycloalkyl, heterocycloalkyl, aryl, heteroaryl, or Y—P_{g}, wherein Y is a linking group and P_{g} is a polymerizable group; R¹⁶ and R¹⁷, when present, are independently at each occurrence C₁-C₆ alkyl, C₁-C₆ alkoxy, C₁-C₆ thioalkyl, C₃-C₇ cycloalkyl, aryl (preferably unsubstituted phenyl or phenyl substituted with alkyl or halo), halo, hydroxy, amino, NR¹⁹R²⁰, or benzyl, wherein R¹⁹ and R²⁰ are independently H or C₁-C₆ alkyl, two adjacent R¹⁶ or R¹⁷ groups, together with the carbon atoms to which they are attached, combine to form a cycloalkyl or aryl ring, Y—P_{g}, or T-Y—P_{g}, wherein T is a bond, O, or NR¹⁸; and EWG is an electron withdrawing group. A preferred EWG is cyano. In some aspects, the ultraviolet-high energy visible light absorber of Formula VI is selected from the group consisting of 3-((9-(dicyanomethylene)-9H-xanthen-3-yl)oxy)propyl methacrylate, 3-(9-(dicyanomethylene)acridin-10(9H)-yl)propyl methacrylate, and combinations thereof. A preferred example of the ultraviolet-high energy visible light absorber of Formula VI is 3-((9-(dicyanomethylene)-9H-xanthen-3-yl)oxy)propyl methacrylate.

In some aspects, the reactive monomer mixtures from which the inventive compositions are made can include at least one light absorbing compound between 0.01 weight percent and 10 weight percent; between 0.01 weight percent and 5 weight percent; between 0.1 weight percent and 1 weight percent; or between 0.1 weight percent and 0.5 weight percent (excluding any diluents).

### Diluents

Any solvent can be used as a diluent to make the inventive compositions. Such diluents can be used to dissolve all of the reactive monomer mixture components (and subsequently removed by evaporation); can be used to plasticize the inventive compositions to reduce, minimize, or eliminate mechanical strain in a crosslinked polymeric network during polymerization; or can be used to control certain physical and mechanical properties such as water content or to control cure kinetics. Common organic solvents can be used as well as primary, secondary, tertiary alcohols, and any combinations thereof. In some aspects, no diluents are used to make the inventive compositions. In other aspects, the reactive monomer mixtures from which the inventive compositions are made can include at least one diluent between 0.01 weight percent and 15 weight percent; between 0.1 weight percent and 10 weight percent; between 0.1 weight percent and 5 weight percent; or between 1 weight percent and 3 weight percent (calculated including the diluents).

### Physical Properties

In some aspects, the inventive compositions can exhibit water contents is less than or equal to 15 weight percent; less than or equal to 10 weight percent; water contents less than or equal to 5 weight percent; or water contents less than or equal to 3 weight percent. In other aspects. the inventive compositions can also exhibit no glistening.

In some aspects, the unexposed (that is, not exposed to UV light) inventive compositions can exhibit Abbe numbers greater than or equal to 25; greater than or equal to 30; greater than or equal to 35; greater than or equal to 40; greater than or equal to 45; or greater than or equal to 50. In other aspects, the unexposed inventive compositions can exhibit refractive indices greater than or equal to 1.40; greater than or equal to 1.45; or greater than or equal to 1.50.

### E. Ophthalmic Devices

In some aspects, the presently disclosed subject matter provides a biomedical or ophthalmic device comprising any of the inventive compositions as described immediately hereinabove.

In particular aspects, the ophthalmic device comprises a lens, inlay, outlay, or insert selected from an intraocular implant, intraocular lens, phakic intraocular lens, contact lens, orthokeratology lens, rigid gas permeable lens, corneal inlay, corneal outlay, or corneal insert. Preferred ophthalmic devices are intraocular implants, intraocular lenses, and phakic intraocular lenses. A more preferred ophthalmic device is an intraocular lens.

In specific aspects, the ophthalmic device is an intraocular implant or lens. More specifically, the presently disclosed subject matter also provides intraocular implants or lenses made at least partially or completely from the inventive compositions described herein. Such intraocular implants or lenses can include an optic portion and one or more haptic portions. In some aspects, the inventive compositions can make up part or all of the optic portion of the intraocular implant or lens. In another aspect, the optic portion of the implant or lens can have a core made from one of the inventive compositions described herein surrounded by another material or polymer. Implants or lenses in which the optic portion is made up of at least partially of one of the inventive compositions disclosed herein can also have a haptic portion. The haptic portion can also be made of the inventive compositions disclosed herein or can be made of a different material, for example, another kind of polymer. In a specific aspect, both the optic portion and the haptic portion of the ophthalmic implant and/or lens are made from one inventive composition. In another aspect, optic portion and the haptic portion of the ophthalmic implant or lens are made from two different inventive compositions.

In some aspects, the intraocular implant or lens of the presently disclosed subject matter is a one-piece lens having a soft, foldable central optic region and an outer peripheral region (haptic-region) in which both regions are made of the same polymer. In other aspects, the optic and haptic regions can be formed from different types of polymers or materials, if desired. Some implants or lenses can also have haptic portions that are made up of different materials, for example, where one or more haptic portions is made from the same material as the optic portion and other haptic portions are made of materials other than inventive compositions disclosed herein. Multicomponent implants or lenses can be made by embedding one material in the other, concurrent extrusion processes, solidifying the hard material about the soft material, or forming an interpenetrating network of the rigid component into a preformed hydrophobic core. In instances where one or more haptic portions are made from a different material than the optic portion of the lens, the haptic portion can be attached to the optic portion in any manner known in the art, such as by drilling a hole or holes in the optic portion and inserting the haptic portion.

The inventive compositions described herein have been designed so that they are capable of being folded so that the intraocular lens can be inserted into the eye of an individual through a small incision. In some instances that incision will be less than 2.5 millimeters; in some instances that incision will be less than 2 millimeters. The haptic portion of the lens provides the required support for the implant or lens in the eye after insertion and unfolding of the lens and tends to help stabilize the position of the lens after insertion and the closure of the incision. The shape of the haptic portion design is not particularly limited and can be any desired configuration, for example, either a plate type or graduated thickness spiral filaments, also known as a C-loop design.

The optic portion of the intraocular lens can be approximately two millimeters to six millimeters in diameter prior to any changes due to hydration. The two millimeters to six millimeter diameter is fairly standard in the art and is generally chosen to cover the pupil in its fully dilated state under naturally occurring conditions. However, other sizes are contemplated and the presently disclosed subject matter is not limited to any particular diameter or size of intraocular lens. Furthermore, it is not necessary that the lens optic portion be circular; it could also be oval, square, or any other shape as desired.

The intraocular lens can further include one or more non-optical haptic components extending away from the outermost peripheral surface of the optic portion. The haptic components can be of any desired shape, for example, graduated spiral filaments or flat plate sections and are used to support the lens within the posterior chamber of the eye. Lenses having any desired design configuration can be fabricated. Should the intraocular lens include other components besides the optical and haptic portions, such other portions can be made of the same polymers as are the haptic and optic portions, or if desired, another material.

The intraocular implants or lenses may be inserted into the eye in any manner known in the art. For example, the intraocular lens may be folded prior to insertion into the eye using an intraocular lens inserter or by small, thin forceps of the type typically used by ophthalmic surgeons. After the implant or lens is in the targeted location, it is released to unfold. As is well known in the art, typically the crystalline lens that is to be replaced is removed prior to insertion of the intraocular lens. The intraocular lens of the presently disclosed subject matter can be made of a generally physiologically inert soft polymeric material that is capable of providing a clear, transparent, refractive lens body even after folding and unfolding. In some aspects, the foldable intraocular lens of the presently disclosed subject matter can be inserted into any eye by injection whereby the mechanically compliant material is folded and forced through a small tube such as a 1 millimeter to 3-millimeter inner diameter tube.

### F. Methods of Making and Adjusting Ophthalmic Devices

In other aspects, the invention provides a method for making an ophthalmic device, the method comprising: (a) providing an inventive composition disclosed herein; and (b) forming an ophthalmic device. Alternatively, the invention provides a method for making an ophthalmic device, the method comprising: (a) preparing a blank from an inventive composition disclosed herein; and (b) machining an ophthalmic device from the blank. Alternatively, the invention provides a method for making an ophthalmic device, the method comprising: molding the device from an inventive composition disclosed herein. Alternatively, the invention provides a method for making an ophthalmic device, the method comprising: (a) providing an inventive composition disclosed herein in a mold assembly; (b) forming an ophthalmic device; and (c) demolding the ophthalmic device from the mold assembly. Alternatively, the invention provides a method for making an ophthalmic device, the method comprising: (a) providing an inventive composition disclosed herein in a mold assembly; (b) forming an ophthalmic device by a free radical photopolymerization reaction; and (c) demolding the ophthalmic device from the mold assembly. In one aspect, the just mentioned free radical photopolymerization reaction comprises irradiating the mold assembly from the top and the bottom with 435 nanometer light emitting diodes having an intensity profile: 2250 seconds at 5 mW/cm² (2.5 mW/cm² top and 2.5 mW/cm² bottom), 2250 seconds at 10 mW/cm² (5 mW/cm²; top and 5 mW/cm² bottom), and 4500 seconds at 20 mW/cm² (10 mW/cm² top and 10 mW/cm² bottom). In other aspects, light emitting diodes of different wavelengths can be used in the aforementioned free radical photopolymerization reaction, for instance, light emitting diodes having average wavelengths of 420 nanometers or 405 nanometers. In yet other aspects, light emitting diodes having different average wavelengths can be used, for example, but not limited to, using 435 nanometer light emitting diodes on the top panels and 405 nanometer light emitting diodes on the bottom panels. In some other aspects, the just mentioned free radical photopolymerization reaction is conducted between 40° C. and 80° C.; between 40° C. and 60° C.; or between 40° C. and 50° C. A preferred free radical photopolymerization temperature is 45° C. In yet other aspects, the just mentioned free radical photopolymerization reaction is conducted in a low oxygen gas atmosphere, wherein the oxygen gas concentration is less than or equal to 2 percent; less than or equal to 1 percent; less than or equal to 0.5 percent; or less than or equal to 0.1 percent. Alternatively, the invention provides a method for making an ophthalmic device, the method comprising: (a) providing an inventive composition disclosed herein in a mold assembly; (b) forming an ophthalmic device by a thermal free radical polymerization reaction; and (c) demolding the ophthalmic device from the mold assembly. The just mentioned thermal free radical polymerization is conducted at temperatures between 40° C. and 120° C.; between 40° C. and 100° C., or between 40° C. and 90° C. The thermal free radical polymerization can be conducted in stages at different temperatures for different times, for instance, 45° C. for about 24 hours followed by 90° C. for about 20 hours. The just mentioned thermal free radical polymerization is conducted in a low oxygen gas atmosphere, wherein the oxygen gas concentration is less than or equal to 2 percent; less than or equal to 1 percent; less than or equal to 0.5 percent; or less than or equal to 0.1 percent.

In other aspects, the above methods can further comprise a step of polishing or lathing the lens surface of a manufactured ophthalmic device or lens. In certain aspects of any of the disclosed methods, the method can further comprise a step of extracting the ophthalmic device with a solvent. Preferred solvents include acetonitrile, isopropanol, ethyl acetate, ethanol, and mixtures thereof, and aqueous solutions of acetonitrile or isopropanol or ethanol. In certain aspects of the disclosed methods, the method can further comprise a step of annealing the ophthalmic device with a vacuum oven, heated nitrogen environment, heated dry air environment, or any combination thereof. In some aspects, any disclosed method can further comprise a step of hydrating the extracted ophthalmic device or lens with at least one aqueous solution. In some aspects, any disclosed method herein can further comprise a coating step. The coating step can comprise any known method of coating such as but not limited to dip coating, spray coating, vapor deposition (chemical or physical), spin coating, and various lamination techniques in which a preformed film is attached to the surface of an ophthalmic device or lens by heat, pressure, or adhesive means, and the like. The coating can protect the surface of the ophthalmic device or lens from damage during use, including any surgical procedure. The coating can also absorb ultraviolet and/or high energy visible light to prevent premature or unwanted activation of the photochemically-allowed, intramolecular [2+2]cycloaddition reactions, at least, at some distance from the coating within the ophthalmic device.

In particular aspects, any disclosed method can further comprise an irradiation step using a light source to activate the photochemically-allowed, intramolecular [2+2]cycloadditions reactions, thereby changing the refractive index in the irradiated areas, which can be used to modify the optical properties of the ophthalmic device or lens, as shown generically in Scheme 2, wherein R^{1a} and R^{1b} represent the chemical structures of R¹ on different sides of the double bond involved in the photochemically allowed, intramolecular [2+2]cycloadditions reactions and wherein X is O or NH. In some aspects, the light source of such an irradiation step can be a selective or targeting light source such as but not limited to a laser, LED, or arc lamp. Such light sources may be continuous or pulsed, and may be stationary (with or without a mask), or scanned over the lens using 1 or 2-axis galinometer scanners, a digital light projector, or some other similar means to selectively irradiated areas of the lens. In certain aspects, the light source is a laser, such as a femtosecond laser or diode laser. In certain aspects, the laser intensity and wavelength are selected according to the desired chemical process (e.g., one-photon, two-photon, or multiphoton absorption). In more specific aspects, the laser is a femtosecond laser which can be directed to selective regions of the ophthalmic device or lens. A femtosecond laser can be of a selected wavelength and pulsed at various frequencies. Preferred laser wavelengths for a one-photon absorption are shorter than or equal to 400 nanometers. Preferred laser wavelengths for a two-photon or multi-photon absorption are longer than or equal to 650 nanometers. Selective or targeted irradiation activates the photochemically-allowed, intramolecular [2+2]cycloaddition reactions in selected regions of the inventive composition, thereby changing the refractive index in the selected regions. By changing the refractive index, the power of the ophthalmic device can be modified. In some aspects, any of the methods disclosed herein can further comprise a step of sterilizing the ophthalmic device or lens. The ophthalmic device or lens can be sterilized by any known means such as, but not limited to, autoclaving, e-beam treatment, and ethylene oxide gas exposure. In other aspects, any irradiation step in any of the disclosed methods herein can be performed either before or after sterilization.

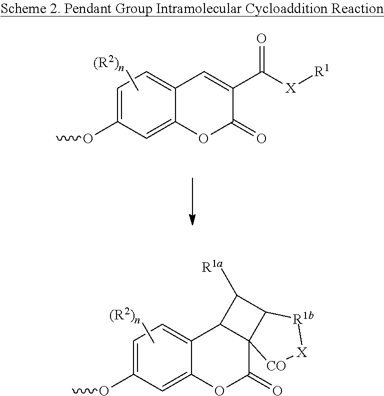

In some aspects, the invention provides a method for adjusting the power of an implanted ophthalmic device, such as an intraocular implant or lens, by selective irradiation of the implanted device, the method comprising: (a) implanting an ophthalmic device made from any one of the inventive compositions in an individual; and (b) adjusting the power of the ophthalmic device by selective irradiation, thereby correcting any vision deficiency. In certain embodiments, the light source for the irradiation step is a selective or targeting light source such as but not limited to a laser, LED, or arc lamp. Such light sources may be continuous or pulsed, and may be stationary (with or without a mask), or scanned over the lens using 1 or 2-axis galinometer scanners, a digital light projector, or some other similar means to selectively irradiated areas of the lens. In certain aspects, the light source is a laser, such as a femtosecond laser or diode laser. In certain aspects, the laser intensity and wavelength are selected according to the desired chemical process (e.g., one-photon, two-photon, or multiphoton absorption). In more specific aspects, the laser is a femtosecond laser which can be directed to selective regions of the ophthalmic device or lens. A femtosecond laser can be of a selected wavelength and pulsed at various frequencies. In other aspects, the implanted ophthalmic device is selected from the group consisting of intraocular implants, intraocular lenses, phakic intraocular lenses, contact lenses, orthokeratology lenses, rigid gas permeable lenses, corneal inlays, corneal outlays, or corneal inserts. Preferred ophthalmic devices are intraocular implants, intraocular lenses and phakic intraocular lenses. A more preferred ophthalmic device is an intraocular lens.

In other aspects, ophthalmic devices made of the inventive composition disclosed herein are coated. As already discussed above, the coating can be an ultraviolet (UV) light absorbing coating and/or an ultraviolet/high energy visible (HEV) light absorbing coating. The UV or UV/HEV light absorbing coating can be on the anterior surface, the posterior surface, or both the anterior and posterior surface of the ophthalmic device. A preferred configuration is having the UV or UV/HEV light absorbing coating only on the anterior surface of the ophthalmic device. A more preferred configuration is having the UV or UV/HEV light absorbing coating on both the anterior and posterior surface of the ophthalmic device due to the ease of manufacturing. The purpose of the UV or UV/HEV light absorbing coating is to prevent the photochemically-allowed, intramolecular [2+2]cycloaddition reactions, at least at some depth within the ophthalmic device, activated by exposure to light during regular use, e.g., by ambient light after implantation of an intraocular lens. The UV or UV/HEV light absorbing coating can also protect the surface of the ophthalmic device or lens from damage during use, including any surgical procedure.

Alternatively, instead of an UV or UV/HEV light absorbing coating, the ophthalmic device made of the inventive compositions disclosed herein can contain high concentrations of UV and/or UV/HEV light absorbers to prevent the photochemically-allowed intramolecular [2+2]cycloaddition reactions, at least at some depth within the ophthalmic device, activated by exposure to light during regular use, e.g., by ambient light after implantation of an intraocular lens. The high concentrations of UV and/or UV/HEV light absorbers can function similarly to a UV or UV/HEV light absorbing coating by absorbing such wavelengths but allowing the ophthalmic device or lens to be modified by a two-photon process, e.g., using a femtosecond laser, at wavelengths unaffected by the high concentrations of UV and/or UV/HEV light absorbers. In some embodiments, ophthalmic devices made of the inventive composition disclosed herein comprise sufficient concentrations of UV and/or UV/HEV light absorbers. In some embodiments, light absorbers are located within the ophthalmic device, evenly distributed throughout the ophthalmic device. In some embodiments, the concentration of UV and/or HEV light absorbers is sufficient to limit the transmission of certain wavelengths of light below a certain depth, also referred to herein as “penetration depth”, from the outer surface of the ophthalmic device. With the attenuation of light, the chromophore present in the outermost layer (forming a “protective layer” the thickness of which corresponds to the “penetration depth”) is allowed to undergo cyclization upon photoexposure, while the chromophore present below the depth of penetration remains unexposed and available to undergo the photochemically-allowed intramolecular [2+2]cycloaddition reactions. In some embodiments, the thickness of the protective layer does not exceed about 500 microns. In some embodiments, the thickness of the protective layer does not exceed about 400 microns. In some embodiments, the thickness of the protective layer does not exceed about 300 microns. In some embodiments, the thickness of the protective layer does not exceed about 200 microns. In some embodiments, the thickness of the protective layer does not exceed about 100 microns. In some embodiments, the thickness of the protective layer does not exceed about 50 microns. In some embodiments, the thickness of the protective layer does not exceed about 25 microns. In some embodiments, the concentration of UV and/or HEV light absorbers in the composition is from about 0.2% to about 5% by weight. In some embodiments, the concentration of UV and/or HEV light absorbers in the composition is from about 0.2% to about 0.5% by weight. In some embodiments, the concentration of UV and/or HEV light absorbers in the composition is from about 0.2% to about 1.0% by weight. In some embodiments, the concentration of UV and/or HEV light absorbers in the composition is from about 0.5% to about 2.5% by weight. In some embodiments, the concentration of UV and/or HEV light absorbers in the composition is from about 2.0% to about 3.0% by weight. In some embodiments, the concentration of UV and/or HEV light absorbers in the composition is from about 3.0% to about 4.0% by weight. In some embodiments, the concentration of UV and/or HEV light absorbers in the composition is from about 4.0% to about 5.0% by weight. While a coating or a protective layer, as described above, may limit the transmission of UV and/or HEV light beyond a certain penetration depth from a surface of the ophthalmic device during regular use (e.g., by ambient light after implantation of an intraocular lens), such protective layer will still allow for adjustments of the ophthalmic device properties beyond the penetration depth of ambient light, via controlled irradiation of the lens material (e.g., refractive index correction via a high-energy laser treatment after implantation of an intraocular lens).

Some aspects of the disclosure will now be described in detail in the following Examples.

### EXAMPLES

Unless otherwise noted, test samples for refractive index, Abbe number, ultraviolet-visible spectroscopy, water content glass transition temperature, and dynamic mechanical analysis were polymer buttons that had been extracted with acetonitrile and dried.

Refractive Index Test Method: Refractive index was measured using an Anton Paar Abbemat WR-wavelength refractometer. The instrument was equilibrated at either 25° C. or 35° C. for a minimum of 1 hour prior to use. The measurement wavelength was set at 589.3 nanometers. Using a pair of tweezers, the sample was placed on the quartz plate. The instrument lid was closed, and the refractive index was recorded after 60 seconds of dwell time. Measurements were performed on three polymer buttons, and the average was reported. In some examples, where it is noted, measurements were performed on both sides of the three polymer buttons, and the average of the six measurements was reported.

Abbe Number Test Method: Following the steps for measuring the refractive index at 589.3 nm, the refractive index at 486.1 nm and 656.3 nm were determined. Measurements were performed on three polymer buttons, and for each polymer button, the refractive index measurements at all three wavelengths were completed before measuring the next replicate. The Abbe number was calculated as follows: V_{D}=(N_{D}−1)/(N_{F}−N_{C}), where N_{D}, N_{F} and N_{C} are the refractive indices of the material at the wavelengths of the Fraunhofer D-, F- and C-spectral lines (589.3 nanometers, 486.1 nanometers and 656.3 nanometers, respectively). The average of the three measurements was reported. In some examples, where it is noted, measurements were performed on both sides of the three polymer buttons, and the average of the six measurements was reported.

Water Content Test Method: The water content (WC) was determined gravimetrically. In this method, three dry polymer discs were individually weighed and transferred to individual glass scintillation vials using sharp-tipped metal tweezers. About 10 mL of HPLC grade water was transferred into each vial, and the samples were incubated at 37° C. for 14 days. After incubation, the polymer discs were removed from the vials using a sharp-tipped metal tweezers and briefly blotted on all sides (flat surfaces and edge) using lint-free blotting paper to remove surface/excess water. Using a dry tweezers, each polymer disc was placed in a tared weighing pan and weighed individually. The water content of the polymer disc was calculated as follows: (% WC)=(wet weight−dry weight)/wet weight×100. The average and standard deviation of the water content were calculated, and the average value reported as the percent water content of the disc.

Ultraviolet-visible spectra of discs formed from the claimed compositions were measured on a Perkin Elmer Lambda 45 UV/VIS or an Agilent Cary 6000i UV/VIS scanning spectrometer as described above using a custom-made, adjustable holder to position the disc in the beam. The custom-made, adjustable holders were V shaped and allowed the discs to slide into place. Baseline correction was performed using empty custom-made, adjustable holders. For obtaining UV-VIS spectra on wet discs, another custom-made, adjustable holder was used to hold cuvettes engineered to hold the disc in the quartz cuvette in the location through which the incident light beam traverses. Baseline correction was performed using custom-made, adjustable holders and empty cuvettes (solvent, no discs). To ensure that the thickness of the samples is constant, all lenses were made using identical molds. Absorbance or transmission spectra are obtained by averaging three individual disc data.

Glass Transition Temperature Test Method: Because of the thickness and/or brittleness of the polymer discs, test samples were cut from the center of the polymer disc using a razor blade. The samples could not be punched out as with a thin film. Test samples were analyzed (in duplicate) on a DSC Q2000 TA instrument at heating rates of 10° C./minute and cooling rates of 5° C./minute under a nitrogen gas atmosphere. The glass transition temperatures were determined from the second heating scans unless noted otherwise.

Dynamic Mechanical Analysis (DMA): Dynamic mechanical analysis was performed using a solids analyzer model RSA G2 from TA Instruments in tension mode. Rectangular specimens were cut from the polymer discs having a width of about 3 millimeters, a length of about 5 millimeters, and a thickness of about 0.75 millimeters. The storage modulus (E′) was determined at 22° C. in the elastic regime, straining at one Hertz. Tan delta (E″/E′) was also determined by a temperature sweep analysis from 10° C. to 40° C. with a temperature sweep rate of 2° C./minute and a strain frequency of one Hertz, wherein E″ is the loss modulus. The units of E′ and E″ are megapascals (MPa). Tan delta is reported as the temperature (° C.) of maximum damping (tan δmax) and is used to estimate the glass transition temperature of the material.

The following abbreviations will be used throughout the Examples and have the following meanings:
LED: light emitting diode(s)
RAM: reactive monomer mixture(s)
RI (25): refractive index measured at 25° C.
RI (35): refractive index measured at 35° C.
Abbe #(25): Abbe number measured at 25° C.
Abbe #(35): Abbe number measured at 35° C.
WC: water content (weight percent)
T_{g}: glass transition temperature (° C.) as determined by differential scanning calorimetry (DSC)
UV/HEV: ultraviolet and/or high energy visible light
TLC: thin layer chromatography
mm: millimeter(s)
cm: centimeter(s)
μm: micrometer(s)
nm: nanometer(s)
L: liter(s)
mL: milliliter(s)
μL: microliter(s)
sec: second(s)
min: minute(s)
hr: hour(s)
mW: milliwatt(s)
J: joule(s)
g: gram(s)
mg: milligram(s)
μg: microgram(s)
mol: mole(s)
mmol: millimole(s)
Eq.: equivalent(s)
g/mol: grams/mole
Da or Dalton(s): gram(s)/mole
kDa: kilodalton(s)
M: molar
mM: millimolar
N: normal (eq/L)
PBS: phosphate buffered saline
DPBS: Dulbecco phosphate buffered saline which contains no calcium or magnesium ions
HCl: hydrochloric acid
DMF: dimethylformamide
ACN: acetonitrile
IPA: 2-propanol
DMSO: dimethyl sulfoxide
EtOAc: ethyl acetate
MeOH: methanol
DCM: dichloromethane or methylene chloride
Et₃N: triethylamine
3E3P: 3-ethyl 3-pentanol
D3O: 3,7-dimethyl-3-octanol (Vigon)
DIW: deionized water
IPA: isopropyl alcohol
PG: 1,2-propylene glycol
PBS: phosphate buffered saline
DIW: deionized water
EDC: 1-(3-dimethylaminopropyl)-3-ethylcarbodiimide hydrochloride
DMAP: N,N-dimethylaminopyridine
pTSA: p-toluenesulfonic acid
CECA: 3,7-dimethyloct-6-en-1-yl 7-((6-(acryloyloxy)hexyl)oxy)-2-oxo-2H-chromene-3-carboxylate (Adesis)

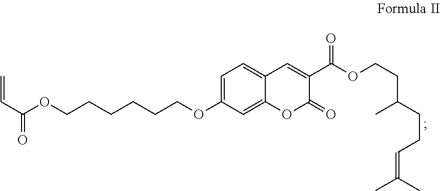 [SMILES: C=CC(=O)OCCCCCCOc1ccc2cc(C(=O)OCCC(C)CCC=C(C)C)c(=O)oc2c1]

HEMA: 2-hydroxyethyl methacrylate (Bimax)
PEGMEMA: poly(ethylene glycol) methyl ether methacrylate (Mₙ=500; Sigma-Aldrich)
EA: ethyl acrylate
EMA: ethyl methacrylate
NBA: n-butyl acrylate
NBMA: n-butyl methacrylate
HBA: 4-hydroxybutyl acrylate [CAS 2478-10-6](TCI or BASF)
NHA: n-hexyl acrylate [CAS 2499-95-8](Sigma-Aldrich)
NHMA: n-hexyl methacrylate
EHMA: 2-ethylhexyl methacrylate
MBDB: 4-(methacryloyloxy)butyl 2,2-dimethylbutanoate

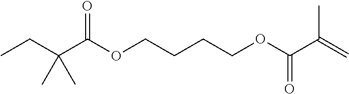 [SMILES: C=C(C)C(=O)OCCCCOC(=O)C(C)(C)CC]

ABDB: 4-(acryloyloxy)butyl 2,2-dimethylbutanoate

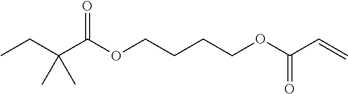 [SMILES: C=CC(=O)OCCCCOC(=O)C(C)(C)CC]

MEBD: 2-(methacryloyloxy)ethyl 2,2-dimethylbutanoate

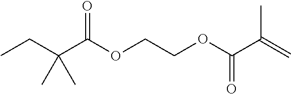 [SMILES: C=C(C)C(=O)OCCOC(=O)C(C)(C)CC]

AEBD: 2-(acryloyloxy)ethyl 2,2-dimethylbutanoate

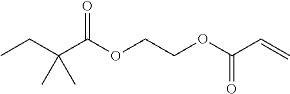

CHA: cyclohexyl acrylate [CAS 3066-71-5](TCI or Alfa Aesar)
CHMA: Cyclohexyl methyl acrylate

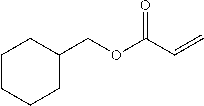

CHEA: 2-Cyclohexylethyl Acrylate

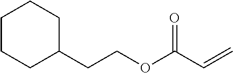

CHPA: 3-Cyclohexylpropyl Acrylate

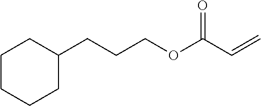

EGDCA: Ethylene glycol dicyclopentenyl ether acrylate [CAS 65983-31-5](Sigma-Aldrich)

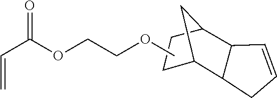

TCDA: Tricyclo[5.2.1.02,6]decanedimethanol diacrylate or dimethylol tricyclo decane diacrylate [CAS 42594-17-2](Sigma-Aldrich or Kyoeisha Chemical Co.)

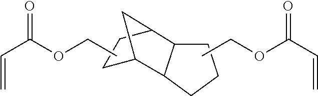

BCHA: ((1R,2S,4R)-bicyclo[2.2.1]hept-5-en-2-yl)methyl acrylate or cyclol acrylate or [(1S,4S)-2-bicyclo[2.2.1]hept-5-enyl]methyl prop-2-enoate [CAS 95-39-6](Monomer-Polymer and DAJAC Labs Inc.)

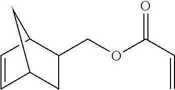

CAA: cinnamyl acrylate

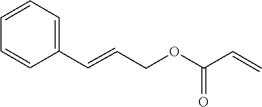

PEA: 2-phenylethyl acrylate [CAS 3530-36-7](MPD)
PEMA: 2-phenylethyl methacrylate [CAS 3683-12-3]
PPA: 3-phenylpropyl acrylate [CAS 85909-41-7]
Omnirad 819: bis(2,4,6-trimethylbenzoyl)-phenylphosphineoxide [CAS 162881-26-7](IGM Resins)
AIBN: azobisisobutyronitrile [CAS 78-67-1]
EGDMA: ethylene glycol dimethacrylate (Esstech)
TMPTMA: trimethylolpropane trimethacrylate (Esstech)
TEGDMA: tetraethylene glycol dimethacrylate (Esstech)
Norbloc: 2-(2′-hydroxy-5-methacrylyloxyethylphenyl)-2H-benzotriazole (Janssen)
UV1: 3-(3-(tert-butyl)-5-(5-chloro-2H-benzo[d][1,2,3]triazol-2-yl)-4-hydroxyphenyl)propyl methacrylate or 2-Methylacrylic acid, 3-[3-tert-butyl-5-(5-chlorobenzotriazol-2-yl)-4-hydroxyphenyl]-propyl ester (Adesis)

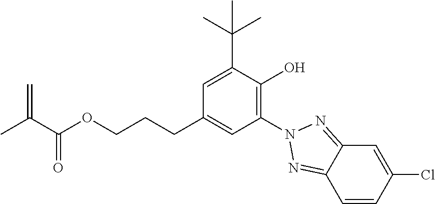 [SMILES: C=C(C)C(=O)OCCCc1cc(-n2nc3ccc(Cl)cc3n2)c(O)c(C(C)(C)C)c1]

UVAM:2-(tert-butyl)-6-(5-chloro-2H-benzo[d][1,2,3]triazol-2-yl)-4-vinylphenol

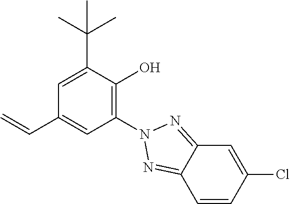 [SMILES: C=Cc1cc(-n2nc3ccc(Cl)cc3n2)c(O)c(C(C)(C)C)c1]

HEV1: 2-(2-cyano-2-(9H-thioxanthen-9-ylidene)acetamido)ethyl methacrylate (Adesis)

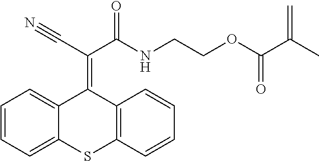 [SMILES: C=C(C)C(=O)OCCNC(=O)C(C#N)=C1c2ccccc2Sc2ccccc21]

HEV2: 3-((9-(dicyanomethylene)-9H-xanthen-3-yl)oxy)propyl methacrylate (Adesis)

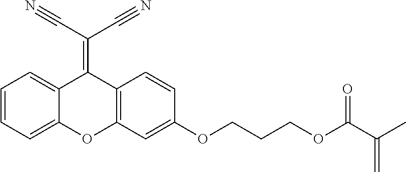 [SMILES: C=C(C)C(=O)OCCCOc1ccc2c(c1)Oc1ccccc1C2=C(C#N)C#N]

HEV3: 2-(2-cyano-2-(9H-xanthen-9-ylidene)acetamido)ethyl methacrylate

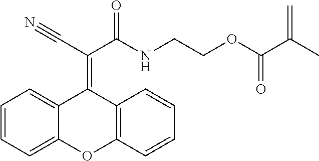 [SMILES: C=C(C)C(=O)OCCNC(=O)C(C#N)=C1c2ccccc2Oc2ccccc21]

HEV4: (E)-2-(2-cyano-2-(3-methoxy-9H-xanthen-9-ylidene)acetamido)ethyl methacrylate

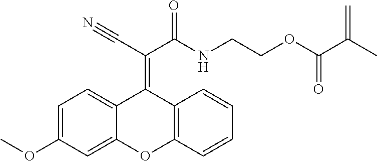 [SMILES: C=C(C)C(=O)OCCNC(=O)/C(C#N)=C1\c2ccccc2Oc2cc(OC)ccc21]

HEV5: (E)-2-(2-(3-butoxy-9H-xanthen-9-ylidene)-2-cyanoacetamido)ethyl methacrylate

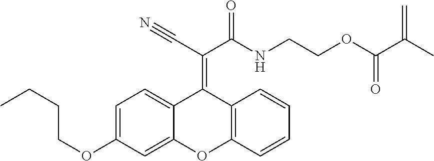 [SMILES: C=C(C)C(=O)OCCNC(=O)/C(C#N)=C1\c2ccccc2Oc2cc(OCCCC)ccc21]

### Preparations

### Synthesis of Cyclohexylmethyl Acrylate (CHMA)

Cyclohexyl methanol (25.0 g, 219.0 mmol) and triethylamine (33.46 g, 330.7 mmol) were dissolved in dichloromethane (450 mL) and cooled to about 0° C. using an ice bath. Acryloyl chloride (29.74 g, 328.5 mmol) was added over a period of 20 minutes while maintaining a constant temperature of about 0° C. After the addition was complete, the reaction mixture was stirred at 0° C. for 30 minutes followed by stirring at ambient temperature overnight. Thin layer chromatography was used to monitor the progress of the reaction. When the reaction was complete, triethylammonium chloride was filtered off, and the filtrate was treated with deionized water (200 mL) and extracted with dichloromethane (3×50 mL). The combined filtrate and organic extracts were washed with water (2×50 mL), brine (25 mL), dried over anhydrous Na₂SO₄, vacuum filtered, and concentrated by rotary evaporation. The crude product was then passed through a short plug of silica gel, eluting with 10% ethyl acetate in n-hexanes, to afford the desired product CHMA as a clear oil (98% yield). ¹H-NMR (500 MHz, CDCl₃): δ 6.39 (1H, dd, J=1.0, 17.0 Hz), 6.12 (1H, dd, J=10.0, 17.0 Hz), 5.81 (1H, dd, J=1.5, 10.0 Hz), 3.97 (2H, d, J=6.0 Hz), 1.76-1.62 (6H, m), 1.31-1.15 (3H, m), 0.95-1.01 (2H, m).

Synthesis of 2-Cyclohexylethyl Acrylate (CHEA): 2-Cyclohexylethyl acrylate was prepared by the same general procedure except that 2-cyclohexyl ethanol was used instead of cyclohexyl methanol (99% yield). ¹H-NMR (500 MHz, CDCl₃): δ 6.38 (1H, dd, J=1.1, 17.2 Hz), 6.11 (1H, dd, J=10.1, 17.2 Hz), 5.80 (1H, dd, J=1.4, 10.1 Hz), 4.18 (2H, t, J=7.0 Hz), 1.74-1.62 (5H, m), 1.58-1.54 (2H, m), 1.39-1.36 (1H, m), 1.27-1.13 (3H, m), 0.97-0.90 (2H, m).

Synthesis of 3-Cyclohexylpropyl Acrylate (CHPA): 3-Cyclohexylpropyl acrylate was prepared by the same general procedure except that 3-cyclohexyl propanol was used instead of cyclohexyl methanol (99% yield). ¹H-NMR (500 MHz, CDCl₃): δ 6.40 (1H, dd, J=1.0, 17.1 Hz), 6.11 (1H, dd, J=10.0, 17.1 Hz), 5.81 (1H, dd, J=1.5, 10.0 Hz), 4.13 (2H, t, J=7.1 Hz), 1.71-1.64 (7H, m), 1.25-1.20 (6H, m), 0.91-0.88 (2H, m).

### Synthesis of 2-(2-cyano-2-(9H-thioxanthen-9-ylidene)acetamido)-ethyl methacrylate is shown in Scheme 3

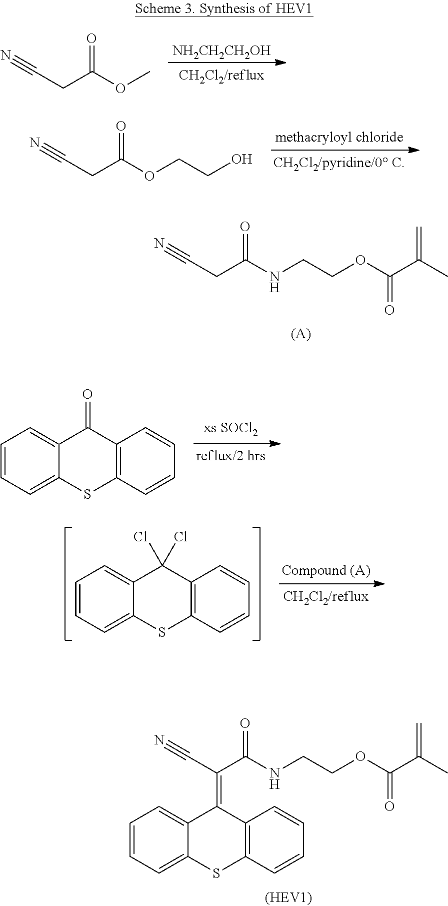

Methyl cyanoacetate (40 grams, 0.4037 mole) and 25 mL of dichloromethane were stirred in a 3 neck, 500 mL round bottom flask under equipped with a reflux condenser under a nitrogen environment. 2-aminoethanol (23.8 grams, 0.3897 mole, ˜0.97 eq.) was added to the solution via an addition funnel, after which the temperature increased, and the methylene chloride began to reflux. After the exotherm ceased, external heat was applied to continue a gentle reflux for a total of two hours, after which no ethanolamine was observed by thin layer chromatography.

The reaction may also be conducted at room temperature and is complete within a few hours.

The mixture was cooled to room temperature and all the methylene chloride was evaporated at reduced pressure. The residual oil was washed three times with 50 mL of ethyl acetate to remove unreacted starting material and non-polar impurities. The residual ethyl acetate was then removed under reduced pressure, and the resulting oil was used for acylation without any further purification.

The crude N-2-hydroxyethylacetamide derivative was dissolved in 150 mL of dichloromethane containing 40 grams of pyridine (˜0.5 mole) in a three-neck round bottom flask equipped with a reflux condenser, an addition funnel, and a magnetic stirring bar. The flask was immersed in an ice bath and allowed to cool down to around 0° C. Methacryloyl chloride (45.76 grams, ˜0.44 mole) was added dropwise from the addition funnel, and the resulting reaction mixture was allowed to warm up to room temperature while constantly stirring the system. Methanol (20 mL) was the added to the flask to quench any unreacted methacryloyl chloride. The volatile components were removed by rotary evaporation under reduced pressure, and the crude product dissolved in 800 mL of dilute aqueous HCl. The resulting aqueous solution was extracted three times with 100 mL of hexanes in a separatory funnel to remove any non-polar impurities. The organic layers were discarded. Sodium chloride was added to the aqueous layer which was then extracted three times with 300 mL of ethyl acetate. About 50 milligrams of BHT were added to the combined organic fractions as an inhibitor, and the ethyl acetate removed by rotary evaporation under reduced pressure. The crude product crystalized out of solution during solvent removal. When about 100 mL of ethyl acetate was left in the flask, 250 mL of hexanes was added, and the crude product was isolated by vacuum filtration using a fritted glass funnel. Thin layer chromatography indicated the presence of a single compound. The filter cake was washed two times with 150 mL of hexanes and then vacuum dried at 40° C., yielding 53 grams (about 70% yield) of 2-(2-cyanoacetamido)ethyl methacrylate (A). ¹H NMR (500 MHz, CDCl₃) δ 1.93 (3H, s, CH₃), 3.36 (2H, s, CNCH₂), 3.60 (2H, dd, CH₂NH), 4.26 (2H, t, CH₂OC═O), 5.59 (1H, m, vinylic), 6.11 (1H, bs, vinylic), 6.52 (1H, bs, NH).

A mixture of 9H-thioxanthene-9-one (2.12 grams, 0.01 mole) and thionyl chloride (5 mL, 8.2 grams, ˜0.07 mole) was refluxed in a 50 mL round bottom flask under a nitrogen atmosphere with constant stirring. After two hours, the red solution was evaporated to dryness ensuring that all unreacted thionyl chloride was removed from the system. 2-(2-Cyanoacetamido)ethyl methacrylate (A) (2.3 grams, 0.0117 mole, ˜1.17 eq.) and 15 mL of dichloromethane were added, and the resulting reaction mixture was heated to reflux under a nitrogen blanket. The reaction was monitored by thin layer chromatography. After two hours, no changes were observed in the chromatogram, and the reaction mixture was allowed to cool to room temperature. 2-(2-cyano-2-(9H-thioxanthen-9-ylidene)acetamido)ethyl methacrylate (HEV1) was isolated as yellow crystals (3.2 grams, 82% yield) after passing through a short silica gel column (CH₂Cl₂, followed by 8 weight % EtOAc in CH₂Cl₂). ¹H NMR (500 MHz, CDCl₃) δ 1.84 (3H, s, CH₃), 3.47 (2H, m, CH₂NH), 4.01 (2H, t, CH₂OC═O), 5.55 (1H, m, vinylic), 5.91 (1H, bs, NH), 5.98 (1H, bs, vinylic), 7.24 (1H, t, Ar—H), 7.31 (1H, t, Ar—H), 7.39 (2H, m, Ar—H), 7.49 (1H, d, Ar—H), 7.55 (1H, m, Ar—H), 7.61 (1H, d, Ar—H), 8.04 (1H, m, Ar—H).

### Synthesis of 3-((9-(dicyanomethylene)-9H-xanthen-3-yl)oxy)propyl methacrylate (HEV2) as shown in Scheme 4

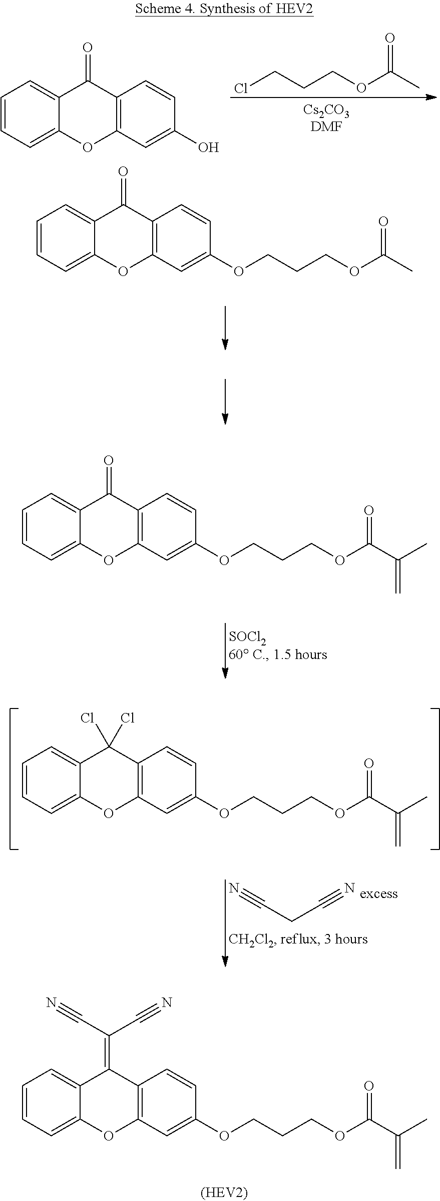

### Synthesis of 3-((9-oxo-9H-xanthen-3-yl)oxy)propyl acetate

A suspension of 3-hydroxy-9H-xanthen-9-one (42.4 grams, 0.2 mole), 70.0 grams Cs₂CO₃ (0.2 mole), and sodium iodide (cat. 200 milligrams) were dried under vacuum in a 500 mL round bottom flask containing a magnetic stirring bar. Anhydrous DMSO (250 mL) was added followed by 2-chloroethyl methacrylate (30.0 grams, 0.2 mole). The reaction mixture was heated overnight at 70° C. Monitoring by TLC indicated complete consumption of the hydroxyxanthenone along with the formation of a less polar derivative. The reaction mixture was cooled to room temperature and slowly poured into dilute aqueous hydrochloric acid with constant stirring. After stirring for thirty minutes, the off-white solid was isolated by vacuum filtration using a fritted glass funnel. The filter cake was washed with deionized water, followed by two washes with 200 mL of hexanes. The 3-((9-oxo-9H-xanthen-3-yl)oxy)propyl acetate was vacuum dried at 60° C. to constant weight.

### Synthesis of 3-((9-oxo-9H-xanthen-3-yl)oxy)propyl alcohol

27 grams of 3-((9-oxo-9H-xanthen-3-yl)oxy)propyl acetate was stirred in about 700 mL of methanol at room temperature, during which 20 mL of 10 N aqueous sodium hydroxide solution was added to the mixture, followed by about 30 mL of deionized water. Monitoring by TLC indicated that the hydrolysis reaction was complete within a few minutes. The mixture was slowly acidified by addition of dilute aqueous hydrochloride acid, after which 150 mL of deionized water was added while constantly stirring the system. The 3-((9-oxo-9H-xanthen-3-yl)oxy)propyl alcohol was isolated by vacuum filtration using a fritted glass funnel, washed with additional amounts of water, and finally dried in a vacuum oven at 60° C.

### Synthesis of 3-((9-oxo-9H-xanthen-3-yl)oxy)propyl methacrylate

25 grams of 3-((9-oxo-9H-xanthen-3-yl)oxy)propanol and 15 mL (10.89 grams) of triethylamine were stirred in 300 mL of anhydrous acetonitrile in a three neck, one liter round bottom flask equipped with a magnetic stirring bar and a reflux condenser. Methacryloyl chloride (9.9 grams) was added to the flask in a dropwise fashion, and mixture was stirred for an hour. The volatile components were evaporated under reduced pressure, and the resulting solids were washed and filtered over a fritted glass funnel and rinsed with deionized water. The residue was washed further with dilute aqueous hydrochloric acid, followed by additional washes with deionized water and finally washed with hexanes. The 3-((9-oxo-9H-xanthen-3-yl)oxy)propyl methacrylate was then dried in a rotary evaporator with bath temperature maintained below 20° C.

### Synthesis of 3-((9-(dicyanomethylene)-9H-xanthen-3-yl)oxy)propyl methacrylate (HEV2)

6.76 grams of 3-((9-oxo-9H-xanthen-3-yl)oxy)propyl methacrylate and 15 mL of thionyl chloride were heated for 2 hours at 65° C. (mantle temperature) in a round bottom flask equipped with a magnetic stirring bar and reflux condenser. The mixture was cooled to room temperature, and the excess thionyl chloride was evaporated under reduced pressure with the bath temperature maintained below 20° C. 3.96 grams of malononitrile was added to the flask, followed by 25 mL of anhydrous dichloromethane, and the mixture was stirred and heated at a gentle reflux for two hours. The mixture was cooled to room temperature and then flushed through a short silica gel plug eluting with methylene chloride and ethyl acetate. Volatile components were evaporated under reduced pressure with the temperature maintained below 20° C., after which the solids were suspended in cold methanol (100 mL) and stirred for 20 minutes. The crude product was isolated by vacuum filtration and the filter cake washed with additional cold methanol. Additional 3-((9-(dicyanomethylene)-9H-xanthen-3-yl)oxy)propyl methacrylate was obtained from the filtrate after evaporation of methanol under reduced pressure, followed by flash chromatography. ¹H NMR (500 MHz, CDCl₃) δ 1.95 (3H, CH₃), 2.25 (2H, m, CH₂), 4.20 (2H, t, CH₂ benzylic), 4.37 (2H, t, CH₂O ester), 5.59 (1H, m, vinylic), 6.12 (1H, m, vinylic), 6.90 (1H, d, Ar—H), 6.97 (1H, dd, Ar—H), 7.40 (1H, ddd, Ar—H), 7.45 (1H, dd, Ar—H), 7.68 (1H, ddd, Ar—H), 8.50 (1H, d, Ar—H), 8.57 (1H, dd, Ar—H).

### WORKING EXAMPLES

### Example 1—Synthesis of 3,7-dimethyloct-6-en-1-yl 7-((6-(acryloyloxy)hexyl)oxy)-2-oxo-2H-chromene-3-carboxylate (CECA) as shown in Scheme 5

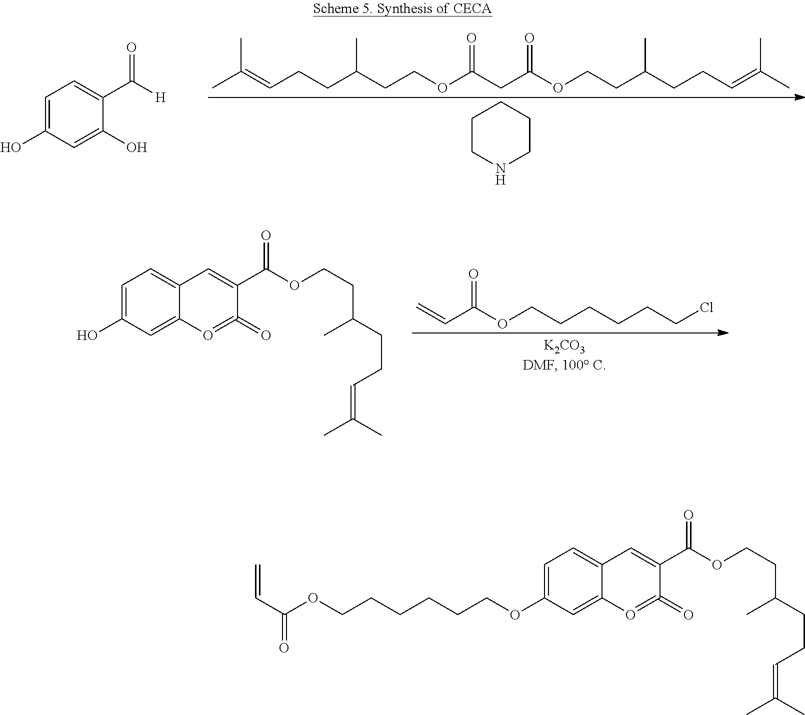

### Bis(3,7-dimethyloct-6-en-1-yl) malonate or Dicitronellyl malonate

Cesium carbonate (2.9 g) was dried under a vacuum while stirring the powder at 170° C. (mantle temperature) for 5 hours. The flask was cooled to room temperature and 100.00 g of citronellol (640 mmol) was added, followed by 40.2 g (304.3) of dimethyl malonate. The flask was placed on a rotary evaporator and rotated under a vacuum with the bath temperature set at 25° C. After 16 hours of stirring, TLC indicated the presence of a small amount of the monocitronellyl ester with the majority of the material being converted to the desired malonate diester. Bath temperature was raised to 35° C. and rotation under vacuum continued until no weight loss was observed in the reaction vessel (0.41 g after 6 hours, 0.55 g after 7.5 hours). The mixture was allowed to stir at 35° C. overnight, additional weight loss of 310 mg was observed from the flask and the reaction appeared complete by TLC. The mixture was cooled to room temperature, 50 mL of hexanes was added to the mixture, which was flushed through a short silica gel plug to remove the cesium carbonate. Volatiles were removed under reduced pressure. The proton NMR spectra of the crude mixture showed the presence of only the desired malonate diester and residual citronellol. ¹H NMR (500 MHz, CDCl₃) δ 5.08 (ddq, J=8.5, 5.7, 1.5 Hz, 1H), 4.23-4.12 (m, 2H), 3.35 (s, 1H), 2.06-1.88 (m, 2H), 1.74-1.64 (m, 4H), 1.60 (d, J=1.4 Hz, 3H), 1.54 (dddd, J=13.1, 7.9, 6.5, 5.2 Hz, 1H), 1.51-1.40 (m, 1H), 1.34 (dddd, J=13.4, 9.6, 6.4, 5.4 Hz, 1H), 1.18 (dddd, J=13.5, 9.5, 7.7, 5.9 Hz, 1H), 0.91 (d, J=6.6 Hz, 3H).

### Synthesis of 6-Chlorohexyl Acrylate

To a stirred and chilled solution of 6-chlorohexanol (50.0 g, 0.37 mol) and triethylamine (50 g, 0.49 mol) in 500 mL of dichloromethane, acryloyl chloride (36.16 g, 0.4 mol) was added in a dropwise fashion while maintaining the reaction temperature at 10° C. or lower at all times. Once the addition was complete, the mixture was allowed to warm up to room temperature while stirring the system for an additional hour. 20 mL of methanol was added to consume any unreacted acryloyl chloride present. Volatiles were evaporated under reduced pressure, organics were dissolved in 500 mL of hexanes and extracted with 3×200 mL of 1 N HCl, followed by brine. Evaporated volatiles under reduced pressure and used the isolated product “as is” for CECA preparation. ¹H NMR (500 MHz, CDCl₃) δ 6.39 (dd, J=17.4, 1.5 Hz, 1H), 6.11 (dd, J=17.3, 10.4 Hz, 1H), 5.81 (dd, J=10.4, 1.5 Hz, 1H), 4.15 (t, J=6.6 Hz, 2H), 3.53 (t, J=6.7 Hz, 2H), 1.83-1.74 (m, 2H), 1.73-1.63 (m, 2H), 1.53-1.35 (m, 4H).

### Synthesis of 3,7-dimethyloct-6-en-1-yl 7-hydroxy-2-oxo-2H-chromene-3-carboxylate

A 50 mL round bottom flask with a magnetic stir bar was charged with 3.76 g of 2,4-dihydroxybenzaldehyde (27.22 mmol), 14.50 g of crude dicitronellyl malonate (1.4 eq, assuming 100% purity) and stirred at room temperature. Piperidine (1.16 g, 0.5 eq) was added in a dropwise fashion while stirring the mixture and the system was heated to 45° C. (mantle temperature). The mixture became clumpy immediately and gradually turned a reddish color over time. Heating was continued at 45° C. while stirring until no change was observed by TLC. The reaction proceeded smoothly, after 1 hour, only the desired coumarin compound was observed as the product being formed. Mantle temperature was increased to 50° C. and stirring continued for 24 hours, after which the reaction appeared complete by TLC. The mixture was cooled to room temperature, and 150 mL of ethyl acetate added prior to extracting the organics with 100 mL of 1 N HCl, followed by brine. Volatiles were evaporated under reduced pressure and the residue was washed with 200 mL of hexanes. A significant amount of the desired product crashed out of solution at this point. The material was filtered over a fritted glass funnel and chromatographed after evaporating all volatiles under reduced pressure. The chromatographed product was added to the previously precipitated solids, redissolved in 15 mL of dichloromethane and 400 mL of hexanes was added to the mixture. The mixture is allowed the mixture to stand for 3 hours, after which, the precipitated product was filtered, washed with warm hexanes (2×50 mL) and dried under vacuum. ¹H NMR (500 MHz, CDCl₃) δ 8.53 (s, 1H), 8.42 (s, 1H), 7.49 (d, J=8.6 Hz, 1H), 7.04 (d, J=2.4 Hz, 1H), 6.93 (dd, J=8.6, 2.3 Hz, 1H), 5.12-5.05 (m, 1H), 4.43-4.31 (m, 2H), 1.99 (th, J=14.8, 6.3 Hz, 4H), 1.87-1.77 (m, 1H), 1.59 (s, 3H), 1.38 (ddt, J=11.7, 9.6, 5.8 Hz, 1H), 1.22 (ddt, J=13.4, 9.3, 6.8 Hz, 1H), 0.96 (d, J=6.3 Hz, 3H).

### Synthesis of 3,7-dimethyloct-6-en-1-yl 7-((6-(acryloyloxy)hexyl)oxy)-2-oxo-2H-chromene-3-carboxylate (CECA)

Potassium carbonate (19.26 g, 139.4 mmol) and sodium iodide (20.09 g, 13.94 mmol) were dried in a 500 mL, 3 neck round bottom flask equipped with a magnetic stir bar and reflux condenser under a vacuum at 140° C. for three hours. The system was cooled to <50° C. and 3,7-dimethyloct-6-en-1-yl 7-hydroxy-2-oxo-2H-chromene-3-carboxylate (16.0 g, 46.46 mmol) was added to the mixture and further dried under vacuum for an additional 30 minutes. The system was placed under a nitrogen blanket and 200 mL of anhydrous DMF added, followed by 9.75 g of 6-chlorohexyl acrylate (51.1 mmol) while constantly stirring the mixture. The mixture was heated at 100° C. (mantle temperature) and the reaction was monitored by TLC. The reaction appeared complete after ˜24 hours, and TLC indicated clean conversion to CECA and the presence of a small amount of its hydrolyzed 6-hydroxy derivative.

The mixture was cooled to room temperature, then 500 mL of 50% ethyl acetate in hexanes it was added, and the combined mixture was poured over 1N HCl. The organics were extracted with deionized water, followed by brine, concentrated under reduced pressure and CECA purified via chromatography on a silica gel column (10% EtOAc in hexanes to 30% EtOAc). Volatiles were removed under reduced pressure, 300 mL hexanes were added to the flask, and the flask placed in a freezer at −20° C. The resultant solids were filtered and washed over a fritted glass funnel and dried under vacuum to yield 21.06 g of CECA (90.9%) as a white solid. ¹H NMR (500 MHz, CDCl₃) δ 8.46 (s, 1H), 7.48 (d, J=8.7 Hz, 1H), 6.87 (dd, J=8.7, 2.4 Hz, 1H), 6.78 (d, J=2.3 Hz, 1H), 6.39 (dd, J=17.4, 1.5 Hz, 1H), 6.12 (dd, J=17.3, 10.4 Hz, 1H), 5.82 (dd, J=10.5, 1.4 Hz, 1H), 5.09 (tp, J=7.1, 1.4 Hz, 1H), 4.42-4.30 (m, 2H), 4.17 (t, J=6.6 Hz, 2H), 4.04 (t, J=6.4 Hz, 2H), 2.08-1.91 (m, 2H), 1.89-1.78 (m, 3H), 1.76-1.68 (m, 2H), 1.67 (t, J=1.3 Hz, 3H), 1.67-1.54 (m, 5H), 1.57-1.42 (m, 3H), 1.39 (dddd, J=13.4, 9.6, 6.4, 5.3 Hz, 1H), 1.22 (dddd, J=13.4, 9.5, 7.5, 6.0 Hz, 1H), 0.96 (d, J=6.4 Hz, 3H).

### Example 2—Synthesis of 3-(3-(2-ethylhexyl)-5-methyl-2,4-dioxo-3,4-dihydropyrimidin-1(2H)-yl)propyl 7-((6-(acryloyloxy)hexyl)oxy)-2-oxo-2H-chromene-3-carboxylate (TECA) as shown in Scheme 7

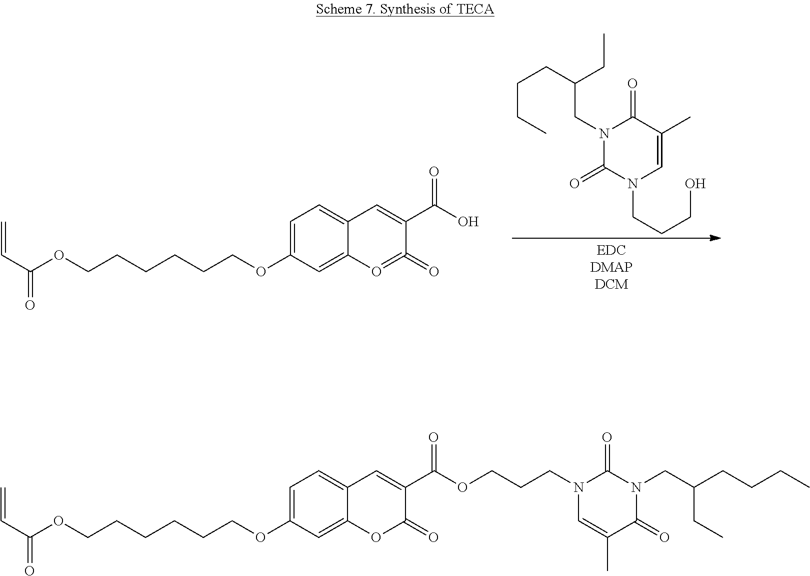

### Synthesis of 7-((6-(acryloyloxy)hexyl)oxy)-2-oxo-2H-chromene-3-carboxylic acid is a multi-step process starting with 2,4-dihydrobezaldehyde as described below

### Synthesis of Ethyl 7-hydroxy-2-oxo-2H-chromene-3-carboxylate

A 500 mL round bottom flask equipped with a magnetic stirrer and reflux condenser was charged with 33.12 g of 2,4-dihydrobezaldehyde (˜24 mol) and 95 g of diethyl malonate (˜2.5 eq.), followed by 15.0 g of piperidine (0.176 mol). The mixture was heated under a nitrogen atmosphere with constant stirring at 55° C. (mantle temperature) and the reaction was monitored by TLC. Once all the aldehyde was consumed, the mixture was cooled to room temperature and poured over ˜500 mL of 4N HCl, stirred for 30 minutes and filtered over a fritted glass funnel. The residual solids were then washed twice with deionized water (300 mL), followed by methanol (3×300 mL) and dried in a vacuum oven at 50° C. The isolated material was used as is for further derivatization. ¹H NMR (500 MHz, DMSO) δ 8.69 (d, J=0.6 Hz, 1H), 7.77 (d, J=8.7 Hz, 1H), 6.86 (dd, J=8.6, 2.3 Hz, 1H), 6.76-6.72 (m, 1H), 4.29 (q, J=7.1 Hz, 2H), 3.20 (s, 1H), 1.32 (t, J=7.1 Hz, 3H).

### Synthesis of Ethyl 2-oxo-7-((6-((tetrahydro-2H-pyran-2-yl)oxy)hexyl)oxy)-2H-chromene-3-carboxylate

Ethyl 7-hydroxy-2-oxo-2H-chromene-3-carboxylate (35 g, ˜0.15 mol), 2-((6-chlorohexyl)oxy)tetrahydro-2H-pyran (36.2 g, 1.1 eq.), and anhydrous potassium carbonate (25.8 g, 1.25 eq.) were suspended in 350 mL of DMSO and the mixture was stirred and heated at a mantle temperature of 90° C. under a nitrogen atmosphere. The reaction appeared complete after 24 hours, after which the mixture was cooled to room temperature, 500 mL of ethyl acetate was added, and the suspension was filtered over a fritted glass funnel. The filtrate was added to 700 mL of water and extracted with 3×350 mL of ethyl acetate. The combined organics were washed with 3×350 mL of water, followed by a brine wash (350 mL), dried over magnesium sulfate, filtered, and volatiles were removed under reduced pressure to provide the crude product as an off-white solid which was used “as is” for hydrolysis of the ethyl ester. ¹H NMR (500 MHz, CDCl₃) δ 8.51 (dd, J=19.7, 1.7 Hz, 1H), 7.51-7.45 (m, 1H), 6.87 (dt, J=8.7, 2.6 Hz, 1H), 6.79 (d, J=2.4 Hz, 1H), 4.57 (p, J=2.2 Hz, 2H), 4.39 (qd, J=7.1, 2.1 Hz, 1H), 4.11 (qd, J=7.2, 1.9 Hz, 2H), 4.04 (tt, J=6.4, 1.4 Hz, 3H), 3.93 (d, J=2.1 Hz, 2H), 3.86 (tt, J=7.9, 2.4 Hz, 2H), 3.80-3.69 (m, 2H), 3.50 (dddt, J=15.0, 6.8, 3.7, 1.7 Hz, 2H), 3.44-3.34 (m, 2H), 2.04 (d, J=1.9 Hz, 3H), 1.88-1.36 (m, 16H), 1.25 (td, J=7.1, 1.9 Hz, 3H).

### Synthesis of 2-oxo-7-((6-((tetrahydro-2H-pyran-2-yl)oxy)hexyl)oxy)-2H-chromene-3-carboxylic acid

Ethyl 2-oxo-7-((6-((tetrahydro-2H-pyran-2-yl)oxy)hexyl)oxy)-2H-chromene-3-carboxylate (60.0 g, ˜0.144 mol) was stirred in 300 mL of ethyl alcohol+180 mL of water, and 57.5 g of 50 weight percent aqueous sodium hydroxide (˜0.718 mol, 5 eq.) was added to the solution. The mixture was heated at a mantle temperature of 40° C. and stirred until all of the ethyl ester was consumed. All volatile components were evaporated under reduced pressure, after which 600 mL of ethyl acetate was added to the mixture prior to acidification using 2 N HCl to a pH of ˜3 while constantly stirring the mixture. Some product precipitated out of solution at this point. The mixture was filtered over a fritted glass funnel and the solids were washed with water. The filtrate was washed with 3 additional batches of water (3×250 mL) and with brine (200 mL), dried over sodium sulfate, filtered, and concentrated under reduced pressure. The combined solids (residue from filtration+dried organic fraction) were then washed with hexanes over a fritted glass funnel and dried in a rotary evaporator, followed by a vacuum oven at 40° C. prior to further derivatization. ¹H NMR (500 MHz, CDCl₃) δ 8.85 (d, J=0.6 Hz, 1H), 7.63 (d, J=8.8 Hz, 1H), 7.00 (dd, J=8.8, 2.4 Hz, 1H), 6.90 (d, J=2.4 Hz, 1H), 4.57 (dd, J=4.6, 2.7 Hz, 1H), 4.09 (t, J=6.5 Hz, 2H), 3.87 (ddd, J=10.9, 7.3, 3.3 Hz, 1H), 3.76 (dt, J=9.6, 6.7 Hz, 1H), 3.54-3.46 (m, 1H), 3.41 (dt, J=9.6, 6.5 Hz, 1H), 1.91-1.83 (m, 2H), 1.86-1.77 (m, 1H), 1.76-1.41 (m, 7H).

### Synthesis of 1-(3-Hydroxypropyl)-3-(2-ethylhexyl)thymine

The synthesis of 1-(3-hydroxypropyl)-3-(2-ethylhexyl)thymine is a multi-step process starting with thymine as described below.

### Synthesis of 1-Acetyl Thymine

Reflux a stirred mixture of 16.0 g thymine (˜0.127 mol) and 100 mL of acetic anhydride under a nitrogen atmosphere for 4 hours. Cool the mixture to room temperature and add 100 mL of a 50/50 mixture of ethyl acetate and hexanes. Filter the solids formed and wash twice with 100 mL of a 75/25 ethyl acetate/hexanes mixture. Dry the product in a vacuum oven at 40° C. and store in the absence of moisture and avoid contact with any protic substances.

### Synthesis of 3-(2-Ethylhexyl)thymine

Sodium carbonate (64 g, ˜0.606 mol) was charged in a 1 L round bottom flask equipped with a magnetic stirrer and a reflux condenser and dried under vacuum at 100 C (mantle temperature) for 2 hours. After cooling the flask to room temperature, 34 g of N-acetylthymine (˜0.202 mol), 2-ethylhexyl-1-bromo hexane (43 g, ˜0.222 mol) were added to the flask, followed by 350 mL of anhydrous DMF, and the mixture heated and stirred at a mantle temperature of 100 C under a nitrogen atmosphere for 36 hours.

Deionized water 100 mL was added to the mixture, which was then stirred for an additional hour at room temperature. The organics were dissolved in 300 mL of ethyl acetate, followed by extraction with 3×500 mL of 5% aqueous sodium chloride. After concentration of the organics by rotary evaporation, the product was isolated after chromatography over a silica gel column. ¹H NMR (500 MHz, CDCl₃) δ 7.00 (q, J=1.2 Hz, 1H), 4.15-4.07 (m, 1H), 3.94-3.81 (m, 3H), 3.65-3.54 (m, 2H), 3.49 (pd, J=7.3, 4.6 Hz, 1H), 2.89 (t, J=6.2 Hz, 1H), 1.93 (dd, J=4.2, 1.2 Hz, 3H), 1.94-1.85 (m, 1H), 1.88-1.83 (m, 1H), 1.87-1.77 (m, 1H), 1.37-1.17 (m, 6H), 0.93-0.82 (m, 6H). Note: to make the 3-butylthymine, the 2-ethylhexyl bromide is replaced with n-butylbromide.

### Synthesis of 1-(3-Hydroxypropyl)-3-(2-ethylhexyl)thymine

To the crude 3-(2-ethylhexyl)thymine (3.0 g, ˜13 mmol) in a round bottom flask equipped with a magnetic stirrer and a reflux condenser, 5.0 g of cesium carbonate (51.4 mmol) and 30 mL of anhydrous DMF were added, followed by 2.55 g of 3-btomopropyl acetate (˜14 mmol) The mixture was heated and stirred under a nitrogen atmosphere at a mantle temperature of 100° C. and the reaction was monitored by TLC. Upon completion, the mixture was cooled to room temperature and poured into 200 mL of water. Methyl alcohol was added to the mixture, followed by 5.0 g of sodium carbonate and the suspension was stirred overnight at room temperature to hydrolyze the ester. Volatiles were evaporated under reduced pressure, organics were extracted into ethyl acetate and the product was purified the product by flash chromatography. ¹H NMR (500 MHz, CDCl₃) δ 7.00 (q, J=1.2 Hz, 1H), 4.15-4.07 (m, 1H), 3.94-3.81 (m, 3H), 3.65-3.54 (m, 2H), 3.49 (pd, J=7.3, 4.6 Hz, 1H), 2.89 (t, J=6.2 Hz, 1H), 1.93 (dd, J=4.2, 1.2 Hz, 3H), 1.94-1.85 (m, 1H), 1.88-1.83 (m, 1H), 1.87-1.77 (m, 1H), 1.37-1.17 (m, 6H), 0.93-0.82 (m, 6H). Note: to make the 1-(3-hydroxypropyl)-3-(2-butyl)thymine, the 3-(2-ethylhexyl)thymine is replaced with 3-butylthymine.

### Synthesis of 3-(3-(2-ethylhexyl)-5-methyl-2,6-dioxo-3,6-dihydropyrimidin-1(2H)-yl)propyl 7-((6-hydroxyhexyl)oxy)-2-oxo-2H-chromene-3-carboxylate

1-Ethyl-3-(3-dimethylaminopropyl)carbodiimide hydrochloride (EDC·HCl, 7.7 g, ˜40 mmol) was added in batches to a stirred mixture of 2-oxo-7-((6-((tetrahydro-2H-pyran-2-yl)oxy)hexyl)oxy)-2H-chromene-3-carboxylic acid (14.67 g, ˜37.6 mmol), 1-(2-ethylhexyl)-3-(3-hydroxypropyl)-5-methylpyrimidine-2,4(1H,3H)-dione (10.25 g, ˜36.34 mmol), and 200 mg of N,N-dimethylamino pyridine in 150 mL of dichloromethane at room temperature. The mixture was stirred at room temperature and reaction progress monitored by thin layer chromatography. The crude material was flushed through a short silica gel plug to obtain the desired THP protected intermediate, which was deprotected after evaporation of the volatiles from the chromatography. Addition of methanol to the isolate resulted in the precipitation of most of the THP ether, which was separated from the mother liquor. Catalytic amounts of toluenesulfonic acid were added to the precipitated compound after dissolution in a small volume of dichloromethane, and addition of methanol. The mother liquor was treated with a small amount of toluenesulfonic acid and both batches were stirred at room temperature until the deprotection was complete. Each mixture was quenched using triethylamine and evaporated to dryness under reduced pressure. The precipitated portion was dissolved in ethyl acetate and washed with dilute aqueous HCl, dried over sodium sulfate and dried in a rotary evaporator to obtain the desired product. The mother liquor portion was further purified after aqueous acid treatment evaporation of volatiles under reduced pressure and chromatography over a silica gel column.

### Synthesis of 3-(3-(2-ethylhexyl)-5-methyl-2,4-dioxo-3,4-dihydropyrimidin-1(2H)-yl)propyl 7-((6-(acryloyloxy)hexyl)oxy)-2-oxo-2H-chromene-3-carboxylate (TECA)

To a stirred solution of 3-(3-(2-ethylhexyl)-5-methyl-2,6-dioxo-3,6-dihydropyrimidin-1(2H)-yl)propyl 7-((6-hydroxyhexyl)oxy)-2-oxo-2H-chromene-3-carboxylate (16.0 g, ˜27.4 mmol) and triethylamine (3.40 g, ˜34 mmol in 150 mL of chilled dichloromethane, acryloyl chloride (3.08 g, ˜1.25 eq) was added in a dropwise fashion over a few minutes, while constantly stirring the mixture and maintaining the temperature <25 C at all times. Methanol (10 mL) was added to the mixture and volatile evaporated under reduced pressure while maintaining bath temperature below 20 C. The product is isolated after dissolution in ethyl acetate, washing with 1N aqueous HCl, drying over sodium sulfate and filtration through a short silica gel plug. ¹H NMR (500 MHz, CDCl₃) δ 8.52 (s, 1H), 7.56-7.41 (m, 1H), 7.30 (d, J=1.4 Hz, 1H), 6.96-6.82 (m, 1H), 6.82-6.70 (m, 1H), 6.37 (dd, J=17.4, 1.4 Hz, 1H), 6.10 (dd, J=17.4, 10.4 Hz, 1H), 5.80 (dd, J=10.4, 1.5 Hz, 1H), 4.34 (t, J=5.8 Hz, 2H), 4.15 (t, J=6.7 Hz, 2H), 4.04 (t, J=6.4 Hz, 2H), 3.99-3.78 (m, 4H), 2.15 (p, J=6.4 Hz, 2H), 1.97-1.60 (m, 9H), 1.59-1.38 (m, 4H), 1.38-1.08 (m, 8H), 0.85 (dt, J=9.0, 7.1 Hz, 6H).

When 1-(3-hydroxypropyl)-3-(2-butyl)thymine is used instead of 1-(3-hydroxypropyl)-3-(2-ethylhexyl)thymine, 3-(3-butyl-5-methyl-2,4-dioxo-3,4-dihydropyrimidin-1(2H)-yl)propyl 7-((6-(acryloyloxy)hexyl)oxy)-2-oxo-2H-chromene-3-carboxylate is synthesized. ¹H NMR (500 MHz, CDCl₃) δ 8.53 (s, 1H), 7.51 (d, J=8.7 Hz, 1H), 7.30 (q, J=1.2 Hz, 1H), 6.89 (dd, J=8.7, 2.4 Hz, 1H), 6.81 (d, J=2.3 Hz, 1H), 6.40 (dd, J=17.3, 1.5 Hz, 1H), 6.12 (dd, J=17.3, 10.4 Hz, 1H), 5.82 (dd, J=10.4, 1.5 Hz, 1H), 4.39-4.33 (m, 2H), 4.17 (t, J=6.6 Hz, 2H), 4.06 (t, J=6.4 Hz, 2H), 3.99-3.90 (m, 4H), 2.21-2.13 (m, 2H), 1.90 (d, J=1.2 Hz, 3H), 1.88-1.80 (m, 2H), 1.77-1.68 (m, 2H), 1.63-1.41 (m, 5H), 1.36 (h, J=7.4 Hz, 2H), 0.93 (t, J=7.4 Hz, 3H).

### Examples 3-5: CECA and TECA Compositions

Under yellow lighting, reactive monomer mixtures were prepared from the formulations listed in Table 1. All components were accurately weighed into an amber glass container and tightly capped with a PTFE lined screw cap. The container was placed on jar roller and rolled until a homogeneous mixture was obtained. The RMM was filtered through a 0.45 μm PTFE membrane (Pall Corporation, Part #66148) using a luer lock glass syringe and a stainless-steel filter fixture and subsequently degassed using a rotary evaporator at the following conditions: 150 millibars, 130 rpm, room temperature, 30 minutes. The degassed RMM was placed in the RMM mold filling compartment of the glove box (<0.1% oxygen, room temperature); the cap was unscrewed; and the RMM was equilibrated for about 3 minutes before use.

In a glove box with a nitrogen gas atmosphere and less than 0.1 percent oxygen gas (v/v), about 250-300 μL of the reactive mixture were dosed using an Eppendorf pipet at room temperature into the front curve mold made of polypropylene. The back curve mold also made of polypropylene was then placed onto the front curve mold. The cavity formed by the front and back curve molds was in the shape of a disc (alternatively, the cavity can form a lens, for instance, an intraocular lens including haptics). The polypropylene molds were equilibrated for a minimum of twelve hours in the glove box prior to dosing. The assemblies were transferred into an adjacent glove box maintained at about 45° C., and the lenses were cured from the top and bottom for a total of one hundred and fifty minutes using 435 nm LED lights on both sides with the following intensity profile: 2250 seconds at 5 mW/cm² (2.5 mW/cm² top and 2.5 mW/cm² bottom), 2250 seconds at 10 mW/cm² (5 mW/cm²; top and 5 mW/cm² bottom), and 4500 seconds at 20 mW/cm² (10 mW/cm² top and 10 mW/cm² bottom). The polypropylene molds were removed manually, and the resulting polymeric discs were extracted with acetonitrile and dried in a vacuum oven, completely protected from ambient light sources. In the extraction process, the polymer discs were extracted at a ratio of about 20 mL acetonitrile per disc overnight (15-17 hours) on an orbital shaker. The solvent was decanted and replenished with a fresh aliquot of acetonitrile and extracted for about 2 hours. The polymer discs were dried overnight at room temperature, and subsequently placed in a vacuum oven at 65° C. for 4 days. The polymeric discs were tested for refractive index and Abbe number before (unexposed) and after (exposed) being irradiated with 368 nm LED lights at 6 mW/cm² for 3600 seconds, and the results are listed in Table 1.

TABLE 1

| | Ex. 3 | Ex. 4 | Ex. 5 |
|---|---|---|---|
| Components | | | |
| (weight %) | | | |
| CECA | 40 | 50 | 0 |
| TECA | 0 | 0 | 50 |
| EGDCA | 19.85 | 13.2 | 0 |
| HBA | 13.85 | 13 | 18 |
| NBMA | 24 | 22 | 29.7 |
| TCDA | 2 | 1.5 | 0 |
| EGDMA | 0 | 0 | 2 |
| Omnirad 819 | 0.3 | 0.3 | 0.3 |
| Σ Components | 100 | 100 | 100 |
| Properties | | | |
| RI (25) - Unexposed | 1.51996 | 1.52616 | 1.52068 |
| RI (25) - Exposed | 1.51153 | 1.51630 | 1.50606 |
| \| Δ RI \| | 0.0083 | 0.0099 | 0.0146 |
| Abbe # (25) - Unexposed | 35 | 29 | 32 |
| Abbe # (25) - Exposed | 52 | 47 | 52 |
| Δ Abbe | 17 | 18 | 20 |

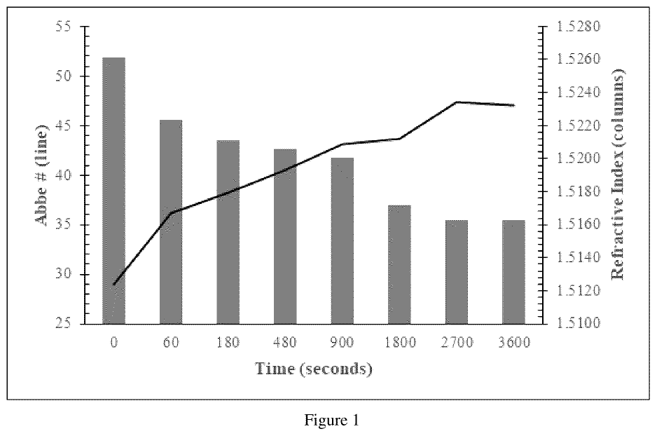

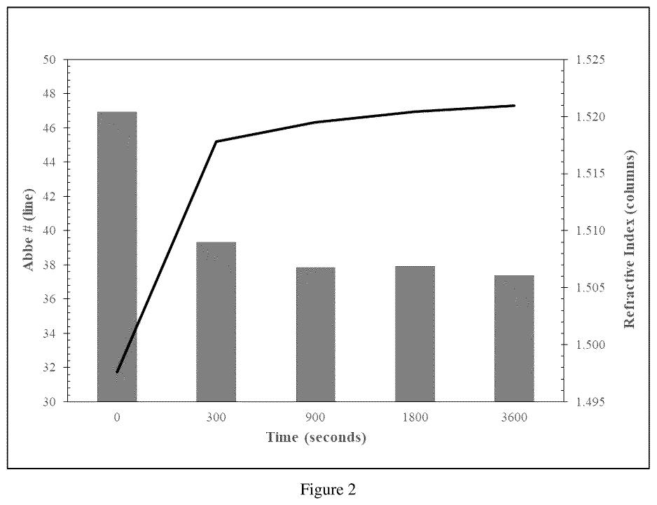

The discs made in Examples 3-5 showed significant decreases in refractive indices upon exposure to ultraviolet light radiation from an LED lamp having an average wavelength of 368 nm and intensity of 6 mW/cm², because of the photochemically-allowed, intramolecular [2+2]cycloaddition reactions (one-photon excitation) in the coumarin pendant groups with concomitant loss of conjugation. For the same reason, the materials made in Examples 3-5 showed significant increases in Abbe number upon exposure to the same ultraviolet radiation. The changes in refractive index and Abbe number as a function of exposure time are shown in FIG. 1 for Example 4 discs (CECA) and FIG. 2 for Example 5 discs (TECA). It is clear from comparing FIGS. 1 and 2 that the CECA material changed gradually over time under the experimental conditions, while the TECA material changed more abruptly during the first 300 seconds and then gradually thereafter.

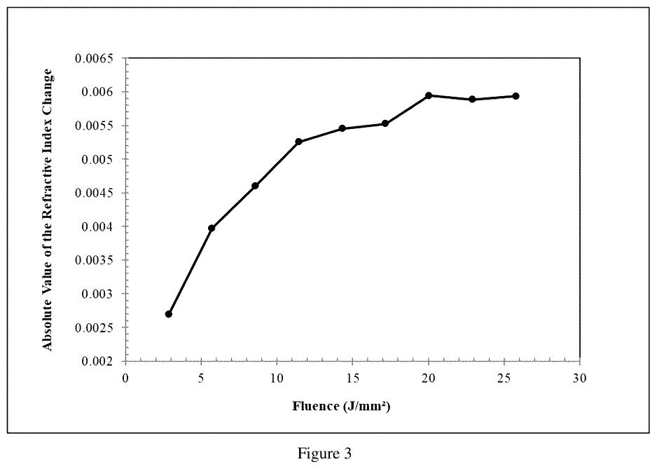

The disc made in Example 3 showed a significant decrease in refractive index upon exposure to visible light irradiation having a wavelength of 715 nm because of the photochemically-allowed, intramolecular [2+2]cycloaddition reactions (two-photon excitation) in the coumarin pendant groups with concomitant loss of conjugation. The absolute value of the change in refractive index (|ΔRI|) as a function of fluence is shown in FIG. 3. It is clear from FIG. 3 that the |ΔRI| depends on the fluence of the irradiation at 715 nm until about 20 Joules/mm² and then appears to level off. In this case, irradiation of the discs was performed by a femtosecond laser [Amplitude, Satsuma] equipped with an optical parametric amplifier [Amplitude, Mango SP or Mango HE] to convert the wavelength from 1030 nm to 715 nm and a digital laser scanning system [Cannon, KP Series]. The pulse energy was about 2 microjoules, the pulse duration was about 250 femtoseconds, the pulse frequency was about 250 kilohertz, and the beam diameter was about 30 micrometers. The scanning speed was about 0.25 micrometers/microsecond. The beam was projected onto the disc in a spiral-like pattern with a distance between treads of the spiral of about one micrometer. The spiral-like pattern was repeated five times.

### Example 6—UV-HEV Protective Coatings

### Synthesis of Poly[3-((9-(dicyanomethylene)-9H-xanthen-3-yl)oxy)propyl methacrylate-co-(2-ethylhexyl acrylate)] or Poly[HEV2-co-2-ethylhexyl acrylate]

A 3 neck, 250 mL round bottom flask equipped with a magnetic stir bar and a reflux condenser was charged with RXY-2 and 2-ethyl hexyl acrylate (5 g each), followed by 50 mL of DMSO. The suspension was stirred while being purged with nitrogen gas to displace dissolved oxygen gas. Thereafter, the apparatus was placed under a nitrogen gas blanket and heated to 75° C. whereupon the suspension became a homogeneous solution. 50 mg of AIBN in 1 mL of degassed DMSO was then added to the mixture. After 30 minutes at 75° C. with stirring, the mantle temperature was raised to 80° C., and the polymerization reaction continued for an additional 9.5 hours.

The resulting mixture was cooled to room temperature and 100 mL of methanol was added while stirring. The liquid was decanted, and residual solids were washed with an additional 100 mL of methanol. The liquid portion was decanted, and another 100 mL of methanol added to the resulting solids. The mixture was heated to reflux with constant stirring. The hot methanol was decanted after 15 minutes of stirring at reflux. This hot methanol washing process was repeated 2 more times. The resulting yellow solid, poly[HEV2-co-2-ethylhexyl acrylate], was dried in a rotary evaporator and used for following coating studies.

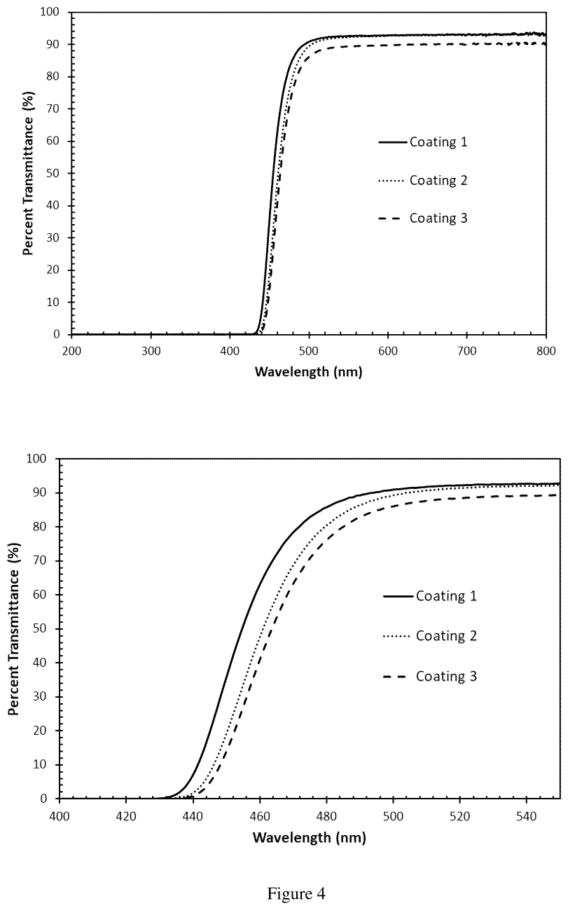

The discs made in Example 3 were coated on a Laurell Model WS-650Mz spin coater under various spin rates using concentrated solutions of aforementioned poly[HEV2-co-2-ethylhexyl acrylate]. The concentrated solutions were dispensed onto the spinning disc which were approximately two microns thick. The UV-VIS transmission spectra of three coated discs are shown in FIG. 4. The coated discs absorb all light at or below 430 nanometers. As a result, such ultraviolet light absorbing coatings can prevent the ultraviolet light, photochemically-allowed, intramolecular [2+2]cycloaddition reactions (one-photon excitation) from occurring at least at some depth within the CECA-containing polymeric discs depending on the absorption characteristics of the coating and the intensity and duration of the ultraviolet light exposure. From a practical point of view, for instance in an implanted, intraocular lens containing polymerized CECA, the ultraviolet light absorbing coating can prevent premature refractive index changes from occurring during normal use (i.e., from sunlight), but allow for optical refinements via a two-photon (TPA) or multi-photon absorption (MPA) process, by using a longer wavelength light source, such as a femtosecond laser.

### Examples 7-11: CECA Compositions

Under yellow lighting, reactive monomer mixtures were prepared from the formulations listed in Table 2. All components were accurately weighed into an amber glass container and tightly capped with a PTFE lined screw cap. The container was placed on jar roller and rolled until a homogeneous mixture was obtained. The RMM was filtered through a 0.45 μm PTFE membrane (Pall Corporation, Part #66148) using a luer lock glass syringe and a stainless-steel filter fixture and subsequently degassed using a rotary evaporator at the following conditions: 150 millibars, 130 rpm, room temperature, 30 minutes. The degassed RMM was placed in the RMM mold filling compartment of the glove box (<0.1% oxygen, room temperature); the cap was unscrewed; and the RMM was equilibrated for about 3 minutes before use.

In a glove box with a nitrogen gas atmosphere and less than 0.1 percent oxygen gas (v/v), about 250-300 μL of the reactive mixture were dosed using an Eppendorf pipet at room temperature into the front curve mold made of polypropylene. The back curve mold also made of polypropylene was then placed onto the front curve mold. The cavity formed by the front and back curve molds was in the shape of a disc (alternatively, the cavity can form a lens, for instance, an intraocular lens including haptics). The polypropylene molds were equilibrated for a minimum of twelve hours in the glove box prior to dosing. The assemblies were transferred into an adjacent glove box maintained at about 45° C., and the lenses were cured from the top and bottom for a total of one hundred and fifty minutes using 435 nm LED lights on both sides with the following intensity profile: 2250 seconds at 5 mW/cm² (2.5 mW/cm² top and 2.5 mW/cm² bottom), 2250 seconds at 10 mW/cm² (5 mW/cm²; top and 5 mW/cm² bottom), and 4500 seconds at 20 mW/cm² (10 mW/cm² top and 10 mW/cm² bottom). The polypropylene molds were removed manually, and the resulting polymeric discs were extracted with acetonitrile and dried in a vacuum oven, completely protected from ambient light sources (“unexposed”). In the extraction process, the polymer discs were extracted at a ratio of about 20 mL acetonitrile per disc overnight (15-17 hours) on an orbital shaker. The solvent was decanted and replenished with a fresh aliquot of acetonitrile and extracted for about 2 hours. The polymer discs were dried overnight at room temperature, and subsequently placed in a vacuum oven at 65° C. for 4 days. The unexposed, polymeric discs were tested for refractive index and Abbe number, and the results are listed in Table 2. These unexposed materials showed high refractive indices of over 1.50 and Abbe numbers over 29 (a few over 34), making them suitable substrates for editable intraocular lenses.

TABLE 2

| | Ex. 7 | Ex. 8 | Ex. 9 | Ex. 10 | Ex. 11 |
|---|---|---|---|---|---|
| Components | | | | | |
| (weight %) | | | | | |
| CECA | 40 | 40 | 40 | 40 | 40 |
| HBA | 18 | 18 | 18 | 18 | 18 |
| NBMA | 39.5 | 0 | 0 | 0 | 0 |
| NHMA | 0 | 39.5 | 0 | 0 | 0 |
| EHMA | 0 | 0 | 39.5 | 0 | 0 |
| ABDB | 0 | 0 | 0 | 39.5 | 0 |
| MEDB | 0 | 0 | 0 | 0 | 39.5 |
| EGDMA | 2 | 2 | 2 | 2 | 2 |
| HEV2 | 0.2 | 0.2 | 0.2 | 0.2 | 0.2 |
| Omnirad 819 | 0.3 | 0.3 | 0.3 | 0.3 | 0.3 |
| Σ Components | 100 | 100 | 100 | 100 | 100 |
| Properties | | | | | |
| RI (25) - Unexposed | 1.509712 | 1.509110 | 1.508340 | 1.509950 | 1.512623 |
| RI (25) Standard | 0.00022 | 0.00029 | 0.00023 | 0.00016 | 0.00026 |
| Deviation | | | | | |
| Abbe # (25) - Unexposed | 33.6617 | 34.1450 | 34.3033 | 32.7450 | 29.3967 |
| Abbe # Standard | 0.9579 | 0.14124 | 0.14067 | 0.10213 | 0.16452 |
| Deviation | | | | | |

### Examples 12, 13, and 14—Thermally Cured CECA Formulations

Under yellow lighting, reactive monomer mixtures were prepared from the formulations listed in Table 3. All components were accurately weighed into an amber glass container and tightly capped with a PTFE lined screw cap. The container was placed on jar roller and rolled until a homogeneous mixture was obtained. The resulting RMM was filtered through a 0.45 μm PTFE membrane (Pall Corporation, Part #66148) using a luer lock glass syringe and a stainless-steel filter fixture and subsequently degassed using a rotary evaporator at the following conditions: 150 millibars, 130 rpm, room temperature, 30 minutes. Enough AIBN was added to the degassed reactive monomer mixtures listed in Table 3 to make the final mixtures contain 0.50 weight percent AIBN. The resulting RMM was placed in the RMM mold filling compartment of the glove box (<0.1% oxygen, room temperature); the cap was unscrewed; and the RMM was equilibrated for about 3 minutes before use.

In a glove box with a nitrogen gas atmosphere and less than 0.1 percent oxygen gas (v/v), about 250-300 μL of the reactive mixture were dosed using an Eppendorf pipet at room temperature into the front curve mold made of polypropylene. The back curve mold also made of polypropylene was then placed onto the front curve mold. The cavity formed by the front and back curve molds was in the shape of a disc (alternatively, the cavity can form a lens, for instance, an intraocular lens including haptics). The polypropylene molds were equilibrated for a minimum of twelve hours in the glove box prior to dosing. The assemblies were transferred into an adjacent glove box containing an oven maintained at about 65° C. and cured for about 24 hours, and then, the temperature was raised to about 90° C., and the formulations cured for another 19 hours. The polypropylene molds were removed manually, and the resulting polymeric discs were extracted with acetonitrile and dried in a vacuum oven, completely protected from ambient light sources (“unexposed”). In the extraction process, the polymer discs were extracted at a ratio of about 20 mL acetonitrile per disc overnight (15-17 hours) on an orbital shaker. The solvent was decanted and replenished with a fresh aliquot of acetonitrile and extracted for about 2 hours. The polymer discs were dried overnight at room temperature, and subsequently placed in a vacuum oven at 65° C. for 4 days. The unexposed, polymeric discs were tested for refractive index and Abbe number, and the results are listed in Table 3. These unexposed materials showed high refractive indices of over 1.51 and Abbe numbers over 29, making them suitable substrates for editable intraocular lenses.

TABLE 3

| | Ex. 12 | Ex. 13 | Ex. 14 |
|---|---|---|---|
| Components | | | |
| (weight %) | | | |
| CECA | 40 | 40 | 40 |
| HBA | 18 | 18 | 18 |
| EMA | 39.8 | 25 | 25 |
| EA | 0 | 14.8 | 0 |
| NBA | 0 | 0 | 14.8 |
| EGDMA | 2 | 2 | 2 |
| HEV2 | 0.2 | 0.2 | 0.2 |
| Σ Components | 100 | 100 | 100 |
| Properties | | | |
| RI (25) - Unexposed | 1.512830 | 1.519262 | 1.518568 |
| RI (25) Standard | 0.000746 | 0.000906 | 0.000743 |
| Deviation | | | |
| Abbe # (25) - Unexposed | 33.36 | 29.24 | 29.68 |
| Abbe # Standard | 0.36 | 0.59 | 0.23 |
| Deviation | | | |

### Examples 15 to 29—Thermally Cured CECA Formulations

Under yellow lighting, reactive monomer mixtures were prepared from the formulations listed in Tables 4-6. All components were accurately weighed into an amber glass container and tightly capped with a PTFE lined screw cap. The container was placed on jar roller and rolled until a homogeneous mixture was obtained. The resulting RMM was filtered through a 0.45 μm PTFE membrane (Pall Corporation, Part #66148) using a luer lock glass syringe and a stainless-steel filter fixture and subsequently degassed using a rotary evaporator at the following conditions: 150 millibars, 130 rpm, room temperature, 30 minutes. Enough AIBN was added to the degassed reactive monomer mixtures listed in Tables 4-6 to make the final mixtures contain 0.50 weight percent AIBN. The resulting RMM was placed in the RMM mold filling compartment of the glove box (<0.1% oxygen, room temperature); the cap was unscrewed; and the RMM was equilibrated for about 3 minutes before use.

In a glove box with a nitrogen gas atmosphere and less than 0.1 percent oxygen gas (v/v), about 250-300 μL of the reactive mixture were dosed using an Eppendorf pipet at room temperature into the front curve mold made of polypropylene. The back curve mold also made of polypropylene was then placed onto the front curve mold. The cavity formed by the front and back curve molds was in the shape of a disc (alternatively, the cavity can form a lens, for instance, an intraocular lens including haptics). The polypropylene molds were equilibrated for a minimum of twelve hours in the glove box prior to dosing. The assemblies were transferred into an adjacent glove box containing an oven maintained at about 65° C. and cured for about 24 hours. The polypropylene molds were removed manually, and the resulting polymeric discs were extracted with acetonitrile and dried in a vacuum oven, completely protected from ambient light sources (“unexposed”). In the extraction process, the polymer discs were extracted at a ratio of about 20 mL acetonitrile per disc overnight (15-17 hours) on an orbital shaker. The solvent was decanted and replenished with a fresh aliquot of acetonitrile and extracted for about 2 hours. The polymer discs were dried overnight at room temperature, and subsequently placed in a vacuum oven at 65° C. for 4 days. The unexposed, polymeric discs were tested for refractive index and Abbe number.

TABLE 4

| Components | | | | | | |
|---|---|---|---|---|---|---|
| (weight %) | Ex. 15 | Ex. 16 | Ex. 17 | Ex. 18 | Ex. 19 | Ex. 20 |
| CECA | 30.99 | 34.98 | 34.4 | 34.82 | 31.61 | 30.81 |
| HBA | 18 | 18 | 18 | 18 | 18 | 18 |
| EMA | 39 | 35.47 | 38.6 | 30.02 | 33.85 | 31.93 |
| NBA | 9.01 | 8.55 | 6.01 | 14.16 | 13.54 | 16.26 |
| EGDMA | 2 | 2 | 2 | 2 | 2 | 2 |
| HEV2 | 0.3 | 0.3 | 0.3 | 0.3 | 0.3 | 0.3 |
| UVAM | 0.7 | 0.7 | 0.7 | 0.7 | 0.7 | 0.7 |
| Σ Components | 100 | 100 | 100 | 100 | 100 | 100 |

TABLE 5

| Components | | | | | | |
|---|---|---|---|---|---|---|
| (weight %) | Ex. 21 | Ex. 22 | Ex. 23 | Ex. 24 | Ex. 25 | Ex. 26 |
| CECA | 34.09 | 34.56 | 32.76 | 27.08 | 27.69 | 29.48 |
| HBA | 18 | 18 | 18 | 18 | 18 | 18 |
| EMA | 32.69 | 26.45 | 29.05 | 35.24 | 37.37 | 34.62 |
| NBA | 12.22 | 17.99 | 17.19 | 16.68 | 13.95 | 14.9 |
| EGDMA | 2 | 2 | 2 | 2 | 2 | 2 |
| HEV2 | 0.3 | 0.3 | 0.3 | 0.3 | 0.3 | 0.3 |
| UVAM | 0.7 | 0.7 | 0.7 | 0.7 | 0.7 | 0.7 |
| Σ Components | 100 | 100 | 100 | 100 | 100 | 100 |

TABLE 6

| Components | | | |
|---|---|---|---|
| (weight %) | Ex. 27 | Ex. 28 | Ex. 29 |
| CECA | 28.24 | 29 | 32.06 |
| HBA | 18 | 18 | 18 |
| EMA | 32.97 | 38.31 | 36.01 |
| NBA | 17.79 | 11.69 | 10.93 |
| EGDMA | 2 | 2 | 2 |
| HEV2 | 0.3 | 0.3 | 0.3 |
| UVAM | 0.7 | 0.7 | 0.7 |
| Σ Components | 100 | 100 | 100 |

### Examples 30 to 35—Thermally Cured CECA Formulations (Prophetic)

Under yellow lighting, reactive monomer mixtures are prepared from the formulations listed in Table 7. All components are accurately weighed into an amber glass container and tightly capped with a PTFE lined screw cap. The container is placed on jar roller and rolled until a homogeneous mixture is obtained. The resulting RMM is filtered through a 0.45 μm PTFE membrane (Pall Corporation, Part #66148) using a luer lock glass syringe and a stainless-steel filter fixture and subsequently degassed using a rotary evaporator at the following conditions: 150 millibars, 130 rpm, room temperature, 30 minutes. Enough AIBN is added to the degassed reactive monomer mixtures listed in Tables 4-6 to make the final mixtures contain 0.50 weight percent AIBN. The resulting RMM is placed in the RMM mold filling compartment of the glove box (<0.1% oxygen, room temperature); the cap is unscrewed; and the RMM is equilibrated for about 3 minutes before use.

In a glove box with a nitrogen gas atmosphere and less than 0.1 percent oxygen gas (v/v), about 255-300 μL of the reactive mixture are dosed using an Eppendorf pipet at room temperature into the front curve mold made of polypropylene. The back curve mold also made of polypropylene is then placed onto the front curve mold. The cavity formed by the front and back curve molds is in the shape of a disc (alternatively, the cavity can form a lens, for instance, an intraocular lens including haptics). The polypropylene molds are equilibrated for a minimum of twelve hours in the glove box prior to dosing. The assemblies are transferred into an adjacent glove box containing an oven maintained at about 65° C. and cured for about 24 hours. The polypropylene molds are removed manually, and the resulting polymeric discs are extracted with acetonitrile and dried in a vacuum oven, completely protected from ambient light sources (“unexposed”). In the extraction process, the polymer discs are extracted at a ratio of about 20 mL acetonitrile per disc overnight (15-17 hours) on an orbital shaker. The solvent is decanted and replenished with a fresh aliquot of acetonitrile and extracted for about 2 hours. The polymer discs are dried overnight at room temperature, and subsequently placed in a vacuum oven at 65° C. for 4 days. The unexposed, polymeric discs are tested for refractive index and Abbe number.

TABLE 7

| Components | | | | | | |
|---|---|---|---|---|---|---|
| (weight %) | Ex. 30 | Ex. 31 | Ex. 32 | Ex. 33 | Ex. 34 | Ex. 35 |
| CECA | 40 | 40 | 40 | 40 | 40 | 40 |
| HBA | 18 | 18 | 18 | 18 | 18 | 18 |
| CHMA | 29 | 19 | 14 | 29 | 19 | 14 |
| PEMA | 0 | 10 | 10 | 0 | 10 | 10 |
| PEGMEMA | 0 | 0 | 5 | 0 | 0 | 5 |
| NBA | 10 | 10 | 10 | 10 | 10 | 10 |
| EGDMA | 2 | 2 | 2 | 0 | 0 | 0 |
| TEGDMA | 0 | 0 | 0 | 2 | 2 | 2 |
| HEV2 | 0.3 | 0.3 | 0.3 | 0.3 | 0.3 | 0.3 |
| UVAM | 0.7 | 0.7 | 0.7 | 0.7 | 0.7 | 0.7 |
| Σ Components | 100 | 100 | 100 | 100 | 100 | 100 |

### Examples 36-52 (More Working Examples)

Under yellow lighting, reactive monomer mixtures were prepared from the formulations listed in Tables 8 and 9. All components were accurately weighed into an amber glass container and tightly capped with a PTFE lined screw cap. The container was placed on jar roller and rolled until a homogeneous mixture was obtained. The resulting RMM was filtered through a 0.45 μm PTFE membrane (Pall Corporation, Part #66148) using a luer lock glass syringe and a stainless-steel filter fixture and subsequently degassed using a rotary evaporator at the following conditions: 150 millibars, 130 rpm, 3° C., 10 minutes. The resulting RMM was placed in the RMM mold filling compartment of the glove box (<0.5% oxygen, room temperature); the cap was unscrewed; and enough AIBN was dissolved into the degassed reactive monomer mixtures listed in Tables 8 and 9 to make the final mixtures contain 0.50 weight percent AIBN.

In a glove box with a nitrogen gas atmosphere and less than 0.5 percent oxygen gas (v/v), about 255-300 μL of the reactive mixture were dosed using an Eppendorf pipet at room temperature into the front curve mold made of polypropylene. The back curve mold also made of polypropylene was then placed onto the front curve mold. The cavity formed by the front and back curve molds was in the shape of a disc (alternatively, the cavity can form a lens, for instance, an intraocular lens including haptics). The polypropylene molds were equilibrated for a minimum of twelve hours in the glove box prior to dosing. The assemblies were transferred into an adjacent glove box containing an oven maintained at about 65° C. and cured for about 24 hours. The polypropylene molds were removed manually, and the resulting polymeric discs were stored in an inert atmosphere and protected from ambient light [denoted as “unexposed, unextracted samples” in the tables].

Some polymeric discs were extracted with acetonitrile and dried in a vacuum oven, completely protected from ambient light sources. In the extraction process, the polymer discs were extracted at a ratio of about 20 mL acetonitrile per disc overnight (15-17 hours) on an orbital shaker. The solvent was decanted and replenished with a fresh aliquot of acetonitrile and extracted for about 2 hours. The polymer discs were dried overnight at room temperature, and subsequently, placed in a vacuum oven at 65° C. for 4 days [denoted as “unexposed, extracted samples” in the tables].

The unexposed, unextracted polymeric discs and the unexposed extracted polymeric discs were tested (six samples for each formulation) for refractive index and Abbe number, and the results are listed in Tables 10 and 11. The RI and Abbe number depended on the CECA content (weight percent) and varies linearly with CECA content (weight percent), which makes the formulation system more predictable in terms of controlling the RI and Abbe number in an ophthalmic device such as an intraocular lens. For the unexposed, unextracted samples, the RI=0.0010*(CECA wt. %)+1.4806 with R²=0.9892, and Abbe number=(−0.4793)*(CECA wt. %)+46.755 with R²=0.9863. For the unexposed, extracted samples, the RI=0.0011*(CECA wt. %)+1.4807 with R²=0.9804, and Abbe number=−(0.4905)*(CECA wt. %)+47.104 with R²=0.9852. As a result, for the unexposed, unextracted samples, RI was also linearly dependent on Abbe number: RI=(−0.0022)*(Abbe #)+1.582 with R²=0.985.

The water content and glass transition temperatures of the unexposed, extracted samples were measured and reported in Table 12. In these experiments, the heating rates and cooling rates were 10° C./minute under a nitrogen gas atmosphere. The glass transition temperatures were determined from the second heating scans. The water contents varied from 1.29 weight percent to 1.65 weight percent water, and the glass transition temperatures varied from 12.9° C. to 19.7° C. These physical properties are desirable to form many ophthalmic devices, such as foldable, glistening free intraocular lenses.

TABLE 8

| | Variable Formulation Components (weight percent) | | | |
|---|---|---|---|---|
| | | | | Σ Variable |
| Example | CECA | EMA | NBA | Components |
| 36 | 23.34 | 42.99 | 14.67 | 81 |
| 37 | 25.44 | 41.95 | 13.61 | 81 |
| 38 | 20.57 | 42.45 | 17.98 | 81 |
| 39 | 22.71 | 41.27 | 17.03 | 81 |
| 40 | 25.95 | 39.51 | 15.55 | 81 |
| 41 | 24.51 | 38.68 | 17.82 | 81 |
| 42 | 26.88 | 36.81 | 17.31 | 81 |
| 43 | 29.08 | 36.00 | 15.92 | 81 |
| 44 | 28.52 | 40.20 | 12.28 | 81 |
| 45 | 27.97 | 38.17 | 14.86 | 81 |
| 46 | 29.92 | 41.08 | 10.00 | 81 |
| 47 | 27.26 | 42.72 | 11.02 | 81 |
| 48 | 30.63 | 37.24 | 13.14 | 81 |
| 49 | 31.90 | 38.55 | 10.55 | 81 |
| 50 | 33.28 | 36.23 | 11.49 | 81 |
| 51 | 25.95 | 39.51 | 15.55 | 81 |
| 52 | 28.52 | 40.20 | 12.28 | 81 |

TABLE 9

| Fixed Formulation Components (weight percent) | Examples 36-52 |
|---|---|
| HEV2 | 0.3 |
| EGDMA | 2 |
| HBA | 16 |
| UVAM | 0.7 |
| Σ Fixed Components | 19 |

TABLE 10
Unexposed, Unextracted Samples

| | | RI (25) | | Abbe # (25) |
|---|---|---|---|---|
| | RI | Standard | Abbe # | Standard |
| Example | (25) | Deviation | (25) | Deviation |
| 36 | 1.505389 | 0.000185 | 35.53 | 0.22 |
| 37 | 1.507558 | 0.000308 | 34.28 | 0.24 |
| 38 | 1.502303 | 0.000257 | 36.95 | 0.36 |
| 39 | 1.504577 | 0.000384 | 35.61 | 0.19 |
| 40 | 1.507502 | 0.000251 | 34.49 | 0.17 |
| 41 | 1.505919 | 0.000179 | 35.00 | 0.09 |
| 42 | 1.507929 | 0.000207 | 34.39 | 0.29 |
| 43 | 1.510260 | 0.000194 | 32.71 | 0.12 |
| 44 | 1.510535 | 0.000162 | 33.04 | 0.14 |
| 45 | 1.509735 | 0.000160 | 33.25 | 0.25 |
| 46 | 1.512179 | 0.000223 | 32.38 | 0.07 |
| 47 | 1.509353 | 0.000306 | 33.86 | 0.50 |
| 48 | 1.512597 | 0.000199 | 31.87 | 0.30 |
| 49 | 1.514299 | 0.000298 | 31.46 | 0.24 |
| 50 | 1.515679 | 0.000140 | 30.77 | 0.11 |
| 51 | 1.507791 | 0.000158 | 34.34 | 0.30 |
| 52 | 1.510762 | 0.000170 | 33.26 | 0.83 |

TABLE 11
Unexposed, Extracted Samples

| | | RI (25) | | Abbe # (25) |
|---|---|---|---|---|
| | RI | Standard | Abbe # | Standard |
| Example | (25) | Deviation | (25) | Deviation |
| 36 | 1.505867 | 0.000230 | 35.77 | 0.35 |
| 37 | 1.508221 | 0.000269 | 34.41 | 0.34 |
| 38 | 1.502779 | 0.000173 | 37.08 | 0.32 |
| 39 | 1.505241 | 0.000245 | 35.81 | 0.32 |
| 40 | 1.507885 | 0.000202 | 34.65 | 0.25 |
| 41 | 1.506444 | 0.000144 | 34.94 | 0.14 |
| 42 | 1.508021 | 0.000106 | 34.41 | 0.13 |
| 43 | 1.510952 | 0.000327 | 32.65 | 0.08 |
| 44 | 1.510968 | 0.000167 | 32.87 | 0.25 |
| 45 | 1.510336 | 0.000140 | 33.44 | 0.17 |
| 46 | 1.513212 | 0.000168 | 32.47 | 0.45 |
| 47 | 1.510194 | 0.000219 | 33.94 | 0.42 |
| 48 | 1.513214 | 0.000100 | 32.00 | 0.15 |
| 49 | 1.514905 | 0.000229 | 31.54 | 0.18 |
| 50 | 1.516382 | 0.000322 | 30.80 | 0.18 |
| 51 | 1.508372 | 0.000771 | 34.14 | 0.40 |
| 52 | 1.511563 | 0.000209 | 33.04 | 0.21 |

TABLE 12
Unexposed, Extracted Samples

| | WC | WC | Glass Transition |
|---|---|---|---|
| | (weight | Standard | Temperature |
| Example | percent) | Deviation | (° C.) |
| 36 | 1.65 | 0.30 | 17.29 |
| 37 | 1.53 | 0.13 | 18.49 |
| 38 | 1.45 | 0.03 | 16.30 |
| 39 | 1.44 | 0.07 | 14.13 |
| 40 | 1.38 | 0.03 | 14.89 |
| 41 | 1.43 | 0.01 | 12.88 |
| 42 | 1.54 | 0.20 | 13.06 |
| 43 | 1.34 | 0.02 | 13.80 |
| 44 | 1.37 | 0.01 | 17.65 |
| 45 | 1.29 | 0.04 | 13.66 |
| 46 | 1.52 | 0.09 | 19.70 |
| 47 | 1.42 | 0.16 | 19.32 |
| 48 | 1.42 | 0.14 | 14.90 |
| 49 | 1.50 | 0.05 | 17.22 |
| 50 | 1.32 | 0.01 | 17.46 |
| 51 | 1.42 | 0.12 | 15.72 |
| 52 | 1.56 | 0.23 | 18.58 |

### Examples 53-55—CECA Discs Comprising UV-HEV Light Absorbers

Under yellow lighting, reactive monomer mixtures were prepared from the formulations listed in Table 13. All components were accurately weighed into an amber glass container and tightly capped with a PTFE lined screw cap. The container was placed on ajar roller and rolled until a homogeneous mixture was obtained. The resulting RMM was filtered through a 0.45 μm PTFE membrane (Pall Corporation, Part #66148) using a luer lock glass syringe and a stainless-steel filter fixture and subsequently degassed using a rotary evaporator at the following conditions: 150 millibars, 130 rpm, 3° C., 10 minutes. The resulting RMM was placed in the RMM mold filling compartment of the glove box (<0.5% oxygen, room temperature); the cap was unscrewed; and the required amount of AIBN was dissolved into the degassed reactive monomer mixture listed in Table 13 to make the final mixture contain 0.50 weight percent AIBN.

TABLE 13

| Components | | | |
|---|---|---|---|
| (weight %) | Ex. 53 | Ex. 54 | Ex. 55 |
| CECA | 30.00 | 35.00 | 34.95 |
| HBA | 18.00 | 18.00 | 18.00 |
| EMA | 36.75 | 34.15 | 34.15 |
| NBA | 12.25 | 9.85 | 9.85 |
| EGDMA | 2.00 | 2.00 | 2.00 |
| RXY-2 | 0.30 | 0.30 | 0.35 |
| UVAM | 0.70 | 0.70 | 0.70 |
| Σ Components | 100 | 100 | 100 |

In a glove box with a nitrogen gas atmosphere and less than 0.5 percent oxygen gas (v/v), about 255-300 μL of the reactive mixture were dosed using an Eppendorf pipet at room temperature into the front curve mold made of polypropylene. The back curve mold also made of polypropylene was then placed onto the front curve mold, forming a cavity between the front and back curve molds in the shape of a disc. The polypropylene molds were equilibrated for a minimum of twelve hours in the glove box prior to dosing. The assemblies were transferred into an adjacent glove box containing an oven maintained at about 65° C., with a nitrogen gas atmosphere with less than 0.5 percent oxygen (v/v), and cured for about 24 hours. The polypropylene molds were removed manually, and the resulting polymeric discs were extracted with acetonitrile and dried in a vacuum oven, completely protected from ambient light sources. In the extraction process, the polymer discs were extracted at a ratio of about 20 mL acetonitrile per disc overnight (15-17 hours) on an orbital shaker. The solvent was decanted and replenished with a fresh aliquot of acetonitrile and extracted for about 2 hours. The polymer discs were dried overnight at room temperature, and subsequently, placed in a vacuum oven at 65° C. for 4 or 6 days.

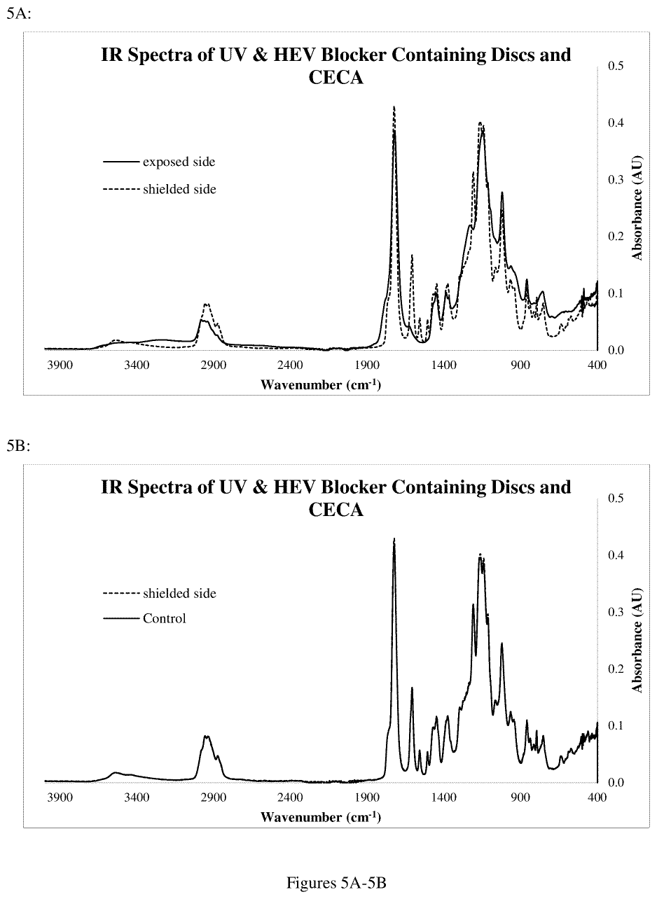

The polymer discs were then placed in an exposure chamber and irradiated on one side (referred to herein as the “exposed side”, the other non-irradiated side being referred to as the “shielded side”) with an OmniCure LX500 spot cure system at a wavelength of 365 nm. Control discs were not irradiated on either side. FIG. 5A compares the infrared spectra of the exposed side and the shielded side of discs prepared as described above, while FIG. 5B shows the infrared spectrum of a shielded side of an irradiated disc, and that of a non-irradiated control disc. The data suggest that exposure to the cure radiation led to consumption of the coumarin present on the exposed side of the disc while the shielded side remained structurally unchanged as compared to the control.

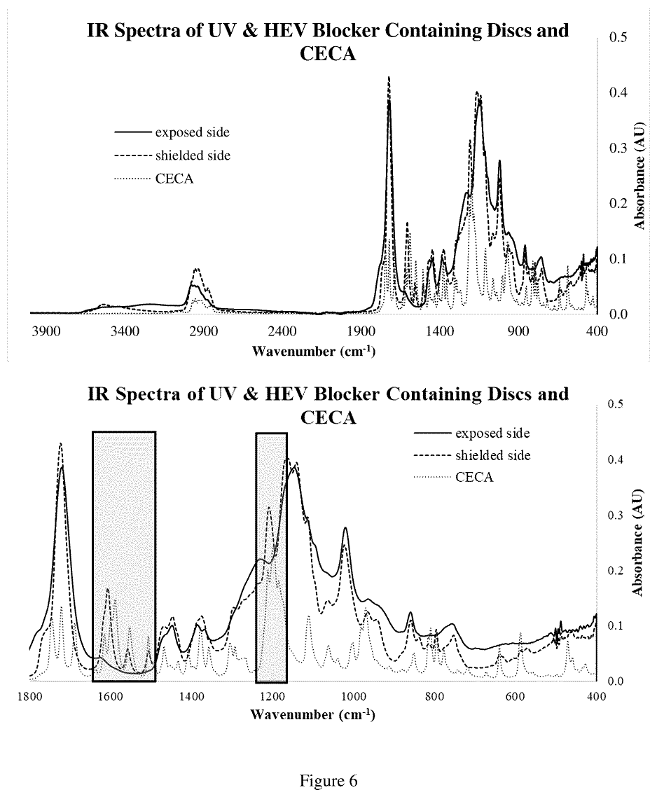

Attenuated Total Reflectance—Infrared Spectroscopy (ATR-IR) studies on the surfaces of shielded and exposed sides of the polymer discs show the disappearance of the C═C stretches between 1615 and 1506 cm⁻¹ and a significant change ˜1210 cm⁻¹ (possibly the C—O sigma bond of the coumarin ester) upon photo-exposure in the exposed side compared to the shielded side which remained completely unchanged relative to the control. The IR spectral overlay in FIG. 6 highlights the C═C stretches and that at ˜1210 cm⁻¹ in polymer discs compared to CECA. The additional signal in the 1615 to 1506 cm⁻¹ region present in CECA is from the C═C stretch of the acryloyl moiety, and is absent in the polymerized material.

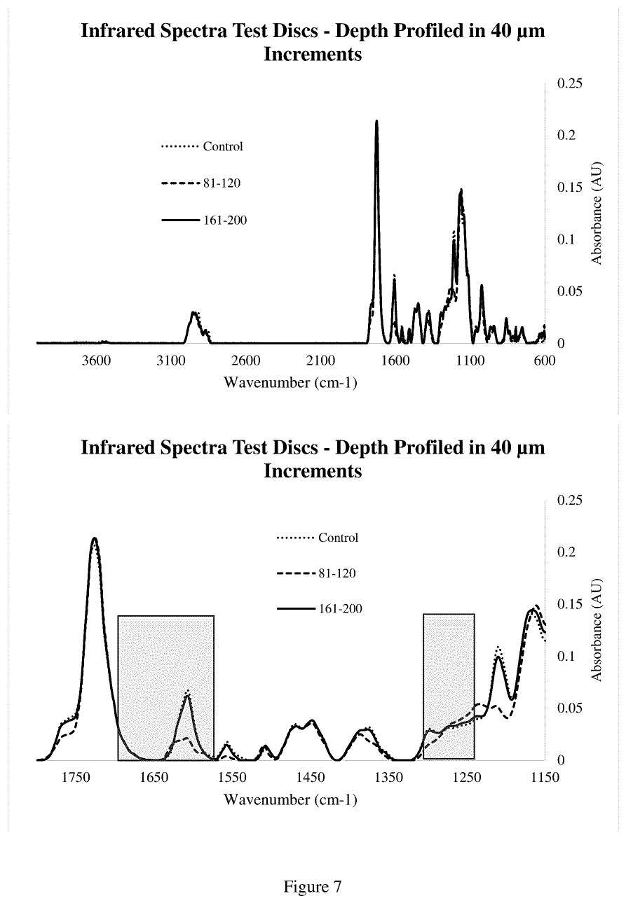

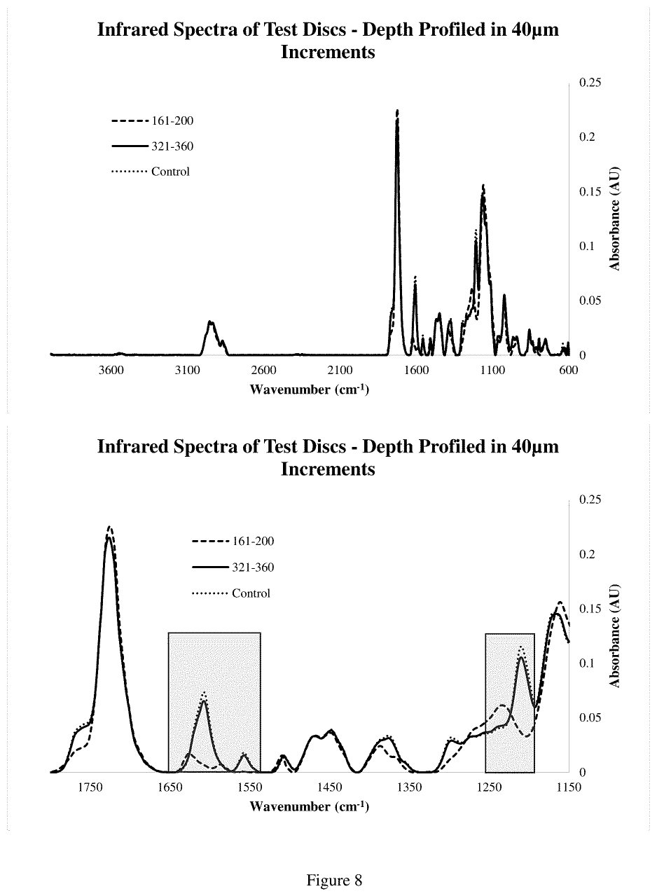

Discs of the same composition were also subjected to radiation on one side of each disc, in a Suntest® CPS+ unit under “daylight reduced IR” conditions at maximum intensity (6.5 mW/cm²) and samples were removed after 7 and 21 days of continuous exposure. Cross-sectional FTIR analysis showed significant cyclization of CECA at both timepoints, but clear attenuation and reduced reaction rates were observed away from the exposed surfaces of discs, as compared to non-irradiated controls. At the 7-day timepoint, samples (FIG. 7) show significant cyclization of CECA up to a depth of ˜160 micrometers from the exposed side surface. At the 21-day timepoint, samples do not show any significant cyclization beyond a depth of 360 nm from the exposed side surface (FIG. 8).

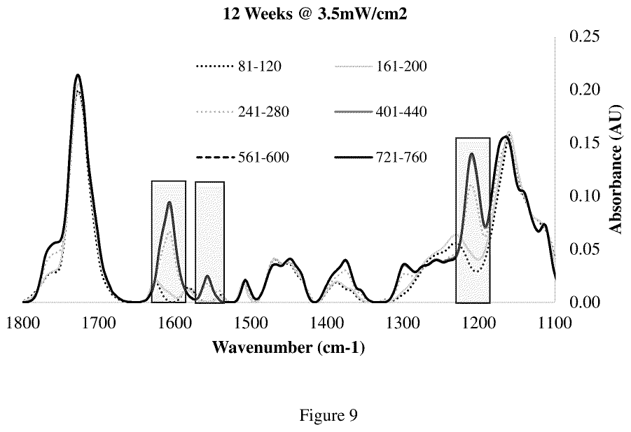

Exposure at a reduced intensity of 3.5 mW/cm² was also evaluated. After 12 weeks of continuous exposure on one side of the disc, the CECA appeared unaffected at depths beyond 400 μm from the exposed surface. FIG. 9 shows the infrared spectra of disc cross-sections obtained in 40 μm increments. Three distinct signals corresponding to the CECA skeleton are observed at ˜1605, 1558, and 1210 cm⁻¹ (FIG. 9). Cyclization of CECA resulted in a loss of the olefinic stretches at 1605 and 1558 cm⁻¹, and a change in the C—O stretch of the ester 1210 cm⁻¹.

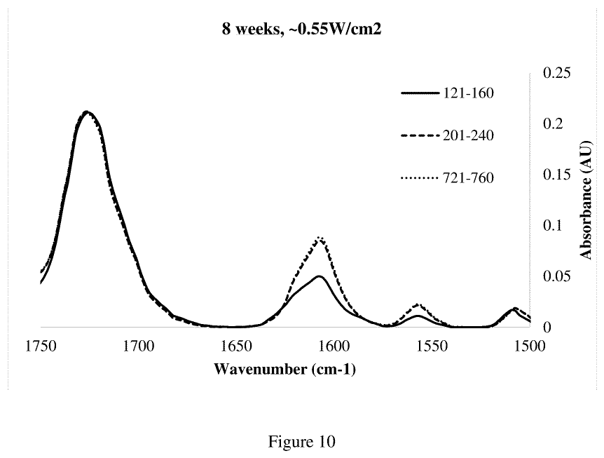

Signal overlap at about 1210 cm⁻¹ does not allow for the use of the stretch in quantifying the extent of cyclization. The 1558 and 1608 cm⁻¹ stretches, on the other hand, are well resolved and may be used in estimating the extent of CECA consumption at various depths. With the total C═O stretches in the formulation (˜1725 cm⁻¹) remaining unchanged, absorbances at 1558 and 1608 cm⁻¹ evaluated against that at 1725 cm⁻¹ are a reasonable indicator of how much CECA has been consumed at different depths from the exposed side surface within the disc. For example, FIG. 10 shows the consumption of CECA in discs of Example 55 after 8 weeks of continuous exposure at 0.55 mW/cm² intensity at 3 different cross-sectional depths of a sample disc.

The absorbances on the surface shielded from light (control side) are 0.2104, 0.0877, and 0.0213 at 1725, 1608, and 1558 cm⁻¹, respectively. These correspond to absorbance ratios of 0.4168, and 0.1012 at these frequencies when normalized to that at 1725 cm⁻¹. The corresponding ratios at depths of 121-160 μm are 0.2369 and 0.0503, and those at the 201-240 μm slice are 0.3989 and 0.959. Ratios approximating the extent of cyclization are shown in Table 14.

TABLE 14
Approximate Residual CECA in polymer discs of Example 55, measured through
comparison of absorbance ratios in irradiated sample discs at varying
depths from the exposed side surface, relative to non-irradiated control
discs. Reported data was averaged over four samples.

| | | | | 1558 cm\hat{ }(−1):1725 | | 1605 cm\hat{ }(−1):1725 | |
|---|---|---|---|---|---|---|---|
| | | | | cm\hat{ }(−1) Absorbance | | cm\hat{ }(−1) Absorbance | |
| | | | | Ratio | | Ratio | |
| | | | | | For | | For |
| | | | | | sample | | sample |
| | | | | | disc | | disc |
| | Absorbance (AU) | | | For | relative to | For | relative to |
| Depth | at 1558 | at 1605 | at 1725 | sample | control | sample | control |
| (μm) | cm\hat{ }(−1) | cm\hat{ }(−1) | cm\hat{ }(−1) | disc | disc | disc | disc |
| 121-160 | 0.0106 | 0.0499 | 0.2106 | 0.0503 | 0.497 | 0.2369 | 0.568 |
| 201-240 | 0.0203 | 0.0844 | 0.2116 | 0.0959 | 0.948 | 0.3989 | 0.957 |
| 721-760 | 0.0213 | 0.0877 | 0.2104 | 0.1012 | 1.000 | 0.4168 | 1.000 |
| (shielded | | | | | | | |
| side) | | | | | | | |

The ratios against those of the controls indicate that approximately 50-55 of the CECA has undergone the cyclization reaction at a depth up to 160 μm, while only about 500 of the compound is lost at a depth between 201 and 240 μm.

Tables 15 and 16 show the relative consumption of CECA under exposure intensities of 3.5 and 6.5 mW/cm\hat{ }2 for 2 to 12 weeks, in discs of Example 53. For calculation purposes, a fraction of 0.95 or above in residual CECA relative to that of the control was considered to be the depth of penetration at a given time point.

TABLE 15
Comparative consumption of CECA - Residual CECA at various depths from the
exposed side surface upon irradiation of discs of Example 53, for 4, 8 or 12 weeks
at 3.5 mW/cm². Reported data was averaged over four samples.

| | Average 1605 cm\hat{ }(−1) | | | | Average 1558 cm\hat{ }(−1) | | | |
|---|---|---|---|---|---|---|---|---|
| | to 1725 cm\hat{ }(−1) | | | | to 1725 cm\hat{ }(−1) | | | |
| | absorbance ratio | | | | absorbance ratio | | | |
| Depth | 12 | 8 | 4 | 2 | 12 | 8 | 4 | 2 |
| (μm) | weeks | weeks | weeks | weeks | weeks | weeks | weeks | weeks |
| 0-40 | 0.0174 | 0.0167 | 0.0253 | 0.0102 | 0.1059 | 0.0438 | 0.0376 | 0.1506 |
| 41-80 | 0.0310 | 0.0149 | 0.0217 | 0.0095 | 0.1659 | 0.0481 | 0.0223 | 0.1534 |
| 81-120 | 0.0171 | 0.0310 | 0.0535 | 0.0093 | 0.0176 | 0.0338 | 0.0876 | 0.1973 |
| 121-160 | 0.1204 | 0.1308 | 0.1146 | 0.9808 | 0.3877 | 0.0828 | 0.0665 | 0.9175 |
| 161-200 | 0.1814 | 0.3307 | 0.3599 | 3.0781 | 0.1914 | 0.2668 | 0.3056 | 1.3647 |
| 201-240 | 0.4094 | 0.5613 | 0.6055 | 1.0526 | 0.3561 | 0.5124 | 0.5500 | 1.0594 |
| 241-280 | 0.4898 | 0.7339 | 0.8051 | 0.9886 | 0.4377 | 0.6949 | 0.7659 | 0.9825 |
| 281-320 | 0.7283 | 0.8424 | 0.9066 | 1.0004 | 0.7131 | 0.8142 | 0.8795 | 0.9928 |
| 321-360 | 0.8174 | 0.9001 | 0.9527 | 1.0068 | 0.7842 | 0.8915 | 0.9394 | 1.0020 |
| 361-400 | 0.8736 | 0.9333 | 0.9730 | 1.0075 | 0.8350 | 0.9242 | 0.9711 | 1.0043 |
| 401-440 | 0.9381 | 0.9560 | 0.9840 | 1.0086 | 0.9123 | 0.9406 | 0.9743 | 1.0026 |
| 441-480 | 0.9426 | 0.9700 | 0.9954 | 1.0082 | 0.9186 | 0.9557 | 0.9898 | 1.0060 |
| 481-520 | 0.9601 | 0.9824 | 0.9991 | 1.0084 | 0.9380 | 0.9683 | 0.9922 | 1.0069 |
| 521-560 | 0.9715 | 0.9922 | 1.0009 | 1.0060 | 0.9563 | 0.9830 | 0.9962 | 1.0078 |
| 561-600 | 0.9820 | 0.9974 | 1.0030 | 1.0025 | 0.9698 | 0.9903 | 0.9977 | 1.0032 |
| 601-640 | 0.9889 | 1.0008 | 1.0055 | 1.0047 | 0.9786 | 1.0039 | 1.0033 | 1.0076 |
| 641-680 | 0.9942 | 1.0011 | 1.0047 | 1.0032 | 0.9861 | 1.0014 | 1.0012 | 1.0035 |
| 681-720 | 1.0008 | 1.0018 | 1.0026 | 1.0015 | 1.0173 | 1.0060 | 0.9999 | 1.0012 |
| 721-760 | 1.0000 | 1.0000 | 1.0000 | 1.0000 | 1.0000 | 1.0000 | 1.0000 | 1.0000 |

TABLE 16
Comparative consumption of CECA - Residual CECA at various
depths from the exposed side surface upon irradiation of discs
of Example 53, for 1 or 3 weeks at 6.5 mW/cm². Reported
data was averaged over four samples.

| | Average 1605 cm\hat{ }(−1) to | | Average 1558 cm\hat{ }(−1) to | |
|---|---|---|---|---|
| | 1725 cm\hat{ }(−1) | | 1725 cm\hat{ }(−1) | |
| Depth | absorbance ratio | | absorbance ratio | |
| (μm) | 3 weeks | 1 week | 3 weeks | 1 week |
| 0-40 | 0.0909 | 0.1300 | 0.5386 | 0.0695 |
| 41-80 | 0.0148 | 0.0961 | 0.1573 | 0.0403 |
| 81-120 | 0.0191 | 0.2861 | 0.0693 | 0.2313 |
| 121-160 | 0.0265 | 0.6814 | 0.0137 | 0.6576 |
| 161-200 | 0.0768 | 0.8609 | 0.0272 | 0.8554 |
| 201-240 | 0.2142 | 0.9342 | 0.1426 | 0.9483 |
| 241-280 | 0.4528 | 0.9649 | 0.3991 | 0.9681 |
| 281-320 | 0.7009 | 0.9782 | 0.6699 | 0.9726 |
| 321-360 | 0.8440 | 0.9857 | 0.8203 | 0.9895 |
| 361-400 | 0.9134 | 0.9927 | 0.9034 | 1.0009 |
| 401-440 | 0.9449 | 0.9991 | 0.9323 | 0.9911 |
| 441-480 | 0.9663 | 1.0025 | 0.9581 | 1.0106 |
| 481-520 | 0.9789 | 1.0015 | 0.9802 | 1.0099 |
| 521-560 | 0.9876 | 1.0041 | 0.9828 | 1.0100 |
| 561-600 | 0.9932 | 1.0081 | 0.9989 | 1.0042 |
| 601-640 | 0.9960 | 1.0068 | 0.9952 | 1.0189 |
| 641-680 | 0.9980 | 1.0056 | 0.9925 | 1.0075 |
| 681-720 | 1.0000 | 1.0000 | 1.0000 | 1.0000 |

| | |
|---|---|
| 721-760 | Not measured |

The data indicate that reducing intensities allows for relatively greater attenuation and improved protection of CECA. The discs exposed at 3.5 mW/cm² appear to have lost ˜5% of CECA at a depth of 450 μm at the end of a 12-week exposure, while the 6.5 mW/cm² exposure resulted in similar consumption at a depth of ˜480 μm at 3 weeks of exposure. Upon exposure at 1.1 mW/cm², discs of Example 54 (containing 0.70% of UVAM and 0.30% of RXY-2 by weight), and Example 55 (containing 0.70% of UVAM and 0.35% of RXY-2 by weight) experienced less than 5% CECA consumption at a depth around 360 μm at 12 weeks of exposure. The marginal increase in RXY-2 content appears to provide a very small improvement in protection for the coumarin (Tables 17 and 18).

TABLE 17
Comparative consumption of CECA - Residual CECA at various
depths from the exposed side surface upon irradiation
of discs of Example 54, for 4, 8 and 12 weeks at 1.1
mW/cm². Reported data was averaged over four samples.

| | Average 1558 cm\hat{ }(−1) | | | Average 1605 cm\hat{ }(−1) | | |
|---|---|---|---|---|---|---|
| | to 1725 cm\hat{ }(−1) | | | to 1725 cm\hat{ }(−1) | | |
| | absorbance ratio | | | absorbance ratio | | |
| Depth | 4 | 8 | 12 | 4 | 8 | 12 |
| (μm) | weeks | weeks | weeks | weeks | weeks | weeks |
| 0-40 | 0.0548 | 0.0000 | 0.0109 | 0.1240 | 0.0000 | 0.0550 |
| 41-80 | 0.0368 | 0.0000 | 0.0452 | 0.0987 | 0.0000 | 0.0580 |
| 81-120 | 0.0810 | 0.0000 | 0.0596 | 0.1504 | 0.0000 | 0.1079 |
| 121-160 | 0.3951 | 0.0000 | 0.2253 | 0.4663 | 0.0000 | 0.2859 |
| 161-200 | 0.7063 | 0.2762 | 0.4214 | 0.7466 | 0.3506 | 0.4928 |
| 201-240 | 0.8834 | 0.6238 | 0.6509 | 0.8982 | 0.6871 | 0.7036 |
| 241-280 | 0.9667 | 0.9006 | 0.8268 | 0.9613 | 0.9246 | 0.8564 |
| 281-320 | 0.9973 | 0.9908 | 0.9178 | 0.9854 | 0.9959 | 0.9346 |
| 321-360 | 1.0084 | 1.0173 | 0.9606 | 0.9931 | 1.0195 | 0.9670 |
| 361-400 | 1.0143 | 1.0289 | 0.9809 | 0.9955 | 1.0272 | 0.9830 |
| 401-440 | 1.0175 | 1.0356 | 0.9896 | 0.9972 | 1.0321 | 0.9909 |
| 441-480 | 1.0212 | 1.0388 | 0.9956 | 0.9990 | 1.0337 | 0.9998 |
| 481-520 | 1.0233 | 1.0427 | 0.9985 | 1.0006 | 1.0349 | 1.0014 |
| 521-560 | 1.0224 | 1.0385 | 1.0006 | 1.0028 | 1.0345 | 1.0042 |
| 561-600 | 1.0220 | 1.0428 | 0.9995 | 1.0033 | 1.0343 | 1.0050 |
| 601-640 | 1.0204 | 1.0261 | 1.0020 | 1.0034 | 1.0235 | 1.0054 |
| 641-680 | 1.0196 | 1.0021 | 1.0025 | 1.0026 | 1.0059 | 1.0049 |
| 681-720 | 1.0130 | 0.9716 | 1.0032 | 1.0000 | 0.9835 | 1.0023 |
| 721-760 | 1.0000 | 1.0773 | 1.0000 | 1.0000 | 1.1085 | 1.0000 |

TABLE 18
Comparative consumption of CECA - Residual CECA at various
depths from the exposed side surface upon irradiation
of discs of Example 55, for 4, 8 and 12 weeks at 1.1
mW/cm². Reported data was averaged over four samples.

| | Average 1558 cm\hat{ }(−1) | | | Average 1605 cm\hat{ }(−1) | | |
|---|---|---|---|---|---|---|
| | to 1725 cm\hat{ }(−1) | | | to 1725 cm\hat{ }(−1) | | |
| | absorbance ratio | | | absorbance ratio | | |
| Depth | 4 | 8 | 12 | 4 | 8 | 12 |
| (μm) | weeks | weeks | weeks | weeks | weeks | weeks |
| 0-40 | 0.0075 | 0.1151 | 0.0511 | 0.0565 | 0.1987 | 0.1201 |
| 41-80 | 0.0137 | 0.0929 | 0.0384 | 0.2993 | 0.1769 | 0.1053 |
| 81-120 | 0.0017 | 0.2575 | 0.0848 | 0.1065 | 0.3387 | 0.1458 |
| 121-160 | 0.2459 | 0.6176 | 0.2690 | 0.3336 | 0.6673 | 0.3403 |
| 161-200 | 0.6866 | 0.8403 | 0.5073 | 0.7262 | 0.8688 | 0.5570 |
| 201-240 | 0.9031 | 0.9798 | 0.6785 | 0.9142 | 0.9955 | 0.7030 |
| 241-280 | 0.9740 | 1.0304 | 0.8288 | 0.9734 | 1.0411 | 0.8346 |
| 281-320 | 0.9993 | 1.0462 | 0.9328 | 0.9931 | 1.0557 | 0.9227 |
| 321-360 | 1.0038 | 1.0558 | 0.9839 | 0.9987 | 1.0625 | 0.9662 |
| 361-400 | 1.0133 | 1.0642 | 1.0027 | 1.0001 | 1.0661 | 0.9845 |
| 401-440 | 1.0149 | 1.0656 | 1.0033 | 1.0034 | 1.0676 | 0.9912 |
| 441-480 | 1.0157 | 1.0669 | 1.0034 | 1.0024 | 1.0672 | 0.9954 |
| 481-520 | 1.0167 | 1.0650 | 1.0076 | 1.0046 | 1.0663 | 0.9987 |
| 521-560 | 1.0149 | 1.0637 | 1.0111 | 1.0036 | 1.0634 | 1.0023 |
| 561-600 | 1.0070 | 1.0621 | 1.0111 | 1.0036 | 1.0581 | 1.0042 |
| 601-640 | 1.0097 | 1.0467 | 1.0111 | 1.0026 | 1.0475 | 1.0045 |
| 641-680 | 1.0046 | 0.9701 | 1.0071 | 1.0021 | 1.0607 | 1.0043 |
| 681-720 | 1.0019 | 1.0230 | 1.0019 | 1.0012 | 1.0247 | 1.0034 |
| 721-760 | 1.0000 | 1.0000 | 1.0000 | 1.0000 | 1.0000 | 1.0000 |

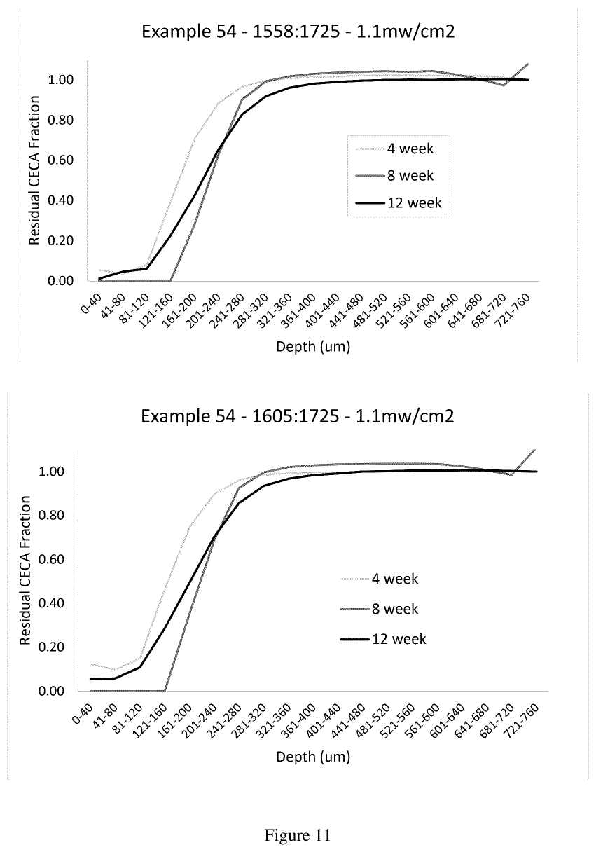

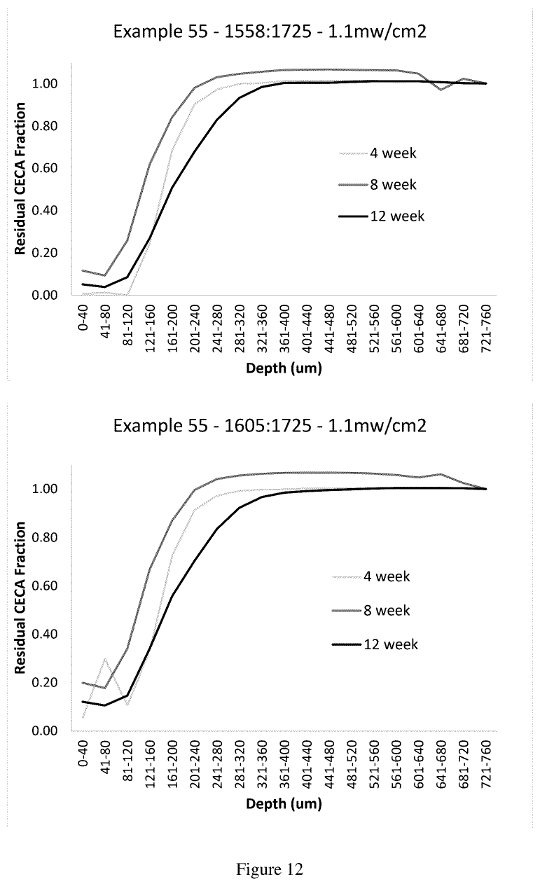

Plots of CECA content along the cross section of one-sided photo-exposed discs of Examples 54 (FIG. 11) and 55 (FIG. 12) indicate significant attenuation and a reduction in conversion rates over time. The 5% CECA consumption penetration depth is ˜280 μm at the end of 4 weeks but is ˜360 μm over the next 8 weeks of exposure.

### Example 56—Vacuum Annealing at 65° C., −30 inHg

Under yellow lighting, reactive monomer mixtures were prepared from the formulation listed in Table 19. All components were accurately weighed into an amber glass container and tightly capped with a PTFE lined screw cap. The container was placed on jar roller and rolled until a homogeneous mixture was obtained. The resulting RMM was filtered through a 0.45 μm PTFE membrane (Pall Corporation, Part #66148) using a luer lock glass syringe and a stainless-steel filter fixture and subsequently degassed using a rotary evaporator at the following conditions: 150 millibars, 130 rpm, room temperature, 30 minutes. Enough AIBN was added to the degassed reactive monomer mixtures listed in Table 19 to make the final mixtures contain 0.50 weight percent AIBN. The resulting RMM was placed in the RMM mold filling compartment of the glove box (<0.1% oxygen, room temperature); the cap was unscrewed; and the RMM was equilibrated for about 3 minutes before use.

In a glove box with a nitrogen gas atmosphere and less than 0.1 percent oxygen gas (v/v), about 250-300 μL of the reactive mixture were dosed using an Eppendorf pipet at room temperature into the front curve mold made of polypropylene. The back curve mold also made of polypropylene was then placed onto the front curve mold. The cavity formed by the front and back curve molds was in the shape of a disc (alternatively, the cavity can form a lens, for instance, an intraocular lens including haptics). The polypropylene molds were equilibrated for a minimum of twelve hours in the glove box prior to dosing. The assemblies were transferred into an adjacent glove box containing an oven maintained at about 65° C. and cured for about 24 hours. The polypropylene molds were removed manually, and the resulting polymeric discs and IOLs were placed in a vacuum oven at about 65° C. and −30 inHg, completely protected from ambient light sources (“unexposed”).

TABLE 19

| Components | |
|---|---|
| (weight %) | Ex. 56 |
| CECA | 27.69 |
| HBA | 18.00 |
| EMA | 37.37 |
| NBA | 13.95 |
| EGDMA | 2.00 |
| HEV2 | 0.30 |
| UVAM | 0.70 |
| Σ Components | 100 |

Samples were harvested a function of time and tested for refractive index and Abbe number and the results are listed in Table 20. These physical properties are desirable to form many ophthalmic devices, such as foldable, glistening free intraocular lenses.

TABLE 20

| | | RI (25) | | Abbe # (25) |
|---|---|---|---|---|
| Number of | RI | Standard | Abbe # | Standard |
| Days | (25) | Deviation | (25) | Deviation |
| 0 | 1.510451 | 0.000253 | 33.20 | 0.20 |
| 1 | 1.510482 | 0.000127 | 33.15 | 0.32 |
| 3 | 1.510057 | 0.000120 | 33.20 | 0.20 |
| 5 | 1.510742 | 0.000221 | 33.26 | 0.25 |
| 8 | 1.511197 | 0.000815 | 33.17 | 0.31 |
| 10 | 1.511143 | 0.000250 | 33.21 | 0.32 |
| 12 | 1.511098 | 0.000129 | 32.97 | 0.29 |

### CLAUSES

Certain aspects of the invention as described hereto can be combined in whole or in part. The following clauses list some non-limiting aspects of the disclosure.

Clause 1. A compound having a chemical structure depicted by Formula I:

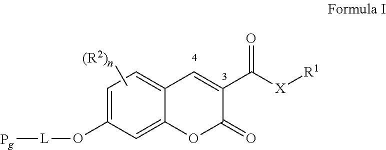

wherein P_{g} is a polymerizable group, L is a linking group, X is O or NH, R¹ is a substituent with at least one double bond capable of undergoing an intramolecular [2+2]cycloaddition reaction with the coumarin double bond in the 3-4 position to form a multi-ring structure including one cyclobutane ring, and R² is independently at each occurrence hydrogen, a C₁-C₆ alkyl group, a C₁-C₆ alkoxy group, a C₁-C₆ thioalkyl group, a C₃-C₇ cycloalkyl group, phenyl, benzyl, halo, hydroxy, amino, or NR³R⁴, wherein R³ and R⁴ are independently hydrogen or a C₁-C₆ alkyl group.

Clause 2. The compound of clause 1 wherein the polymerizable group comprises (meth)acrylate, (meth)acrylamide, N-vinyl lactam, N-vinyl amide, vinyl carbonate, vinyl carbamate, vinyl ether, or styryl.

Clause 3. The compound of clause 2 wherein the polymerizable group is (meth)acrylate.

Clause 4. The compound of any one of clauses 1 to 3 wherein the linking group comprises an alkylene, oxaalkylene, alkyleneoxy, cycloalkylene, heterocycloalkylene, arylene, heteroarylene, alkylene-amide-alkylene, alkylene-amine-alkylene, or combinations thereof.

Clause 5. The compound of clause 4 wherein the linking group is an alkylene having between one and twenty-five carbon atoms.

Clause 6. The compound of clause 5 wherein the alkylene is hexamethylene (CH₂)₆.

Clause 7. The compound of any one of clauses 1 to 6 wherein the R¹ of the compound depicted by Formula I has the chemical structure:

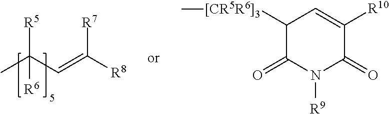

wherein R⁵, R⁶, R⁷, and R⁸ are independently hydrogen or a C₁-C₆ alkyl group, or when R⁷ and R⁸ are connected to form cycloaliphatic ring, the cycloaliphatic ring can contain between five and eight carbon atoms; and wherein R⁹ and R¹⁰ are independently hydrogen or C₁-C₂₀ alkyl groups.

Clause 8. The compound of clause 1 wherein the compound is 3,7-dimethyloct-6-en-1-yl 7-((6-(acryloyloxy)hexyl)oxy)-2-oxo-2H-chromene-3-carboxylate, having the chemical structure shown in Formula II:

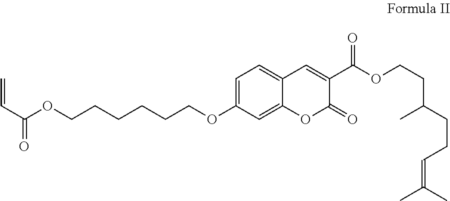 [SMILES: C=CC(=O)OCCCCCCOc1ccc2cc(C(=O)OCCC(C)CCC=C(C)C)c(=O)oc2c1]

Clause 9. The compound of clause 1 wherein the compound is 3-(3-(2-ethylhexyl)-5-methyl-2,4-dioxo-3,4-dihydropyrimidin-1(2H)-yl)propyl-7-((6-(acryloyloxy)hexyl)oxy)-2-oxo-2H-chromene-3-carboxylate, having the chemical structure shown in Formula III:

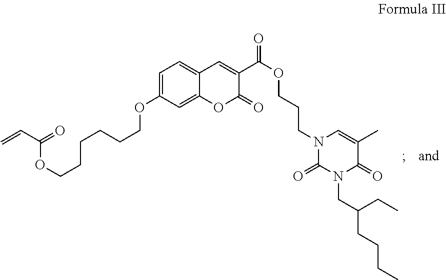 [SMILES: C=CC(=O)OCCCCCCOc1ccc2cc(C(=O)OCCCn3cc(C)c(=O)n(CC(CC)CCCC)c3=O)c(=O)oc2c1]

Clause 10. The compound of clause 1 wherein the compound is 3-(3-butyl-5-methyl-2,4-dioxo-3,4-dihydropyrimidin-1(2H)-yl)propyl-7-((6-(acryloyloxy)hexyl)oxy)-2-oxo-2H-chromene-3-carboxylate, having the chemical structure shown in Formula IV:

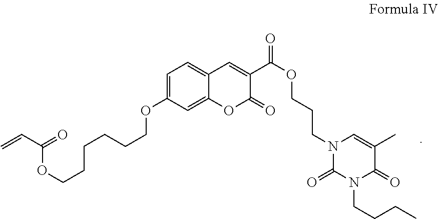 [SMILES: C=CC(=O)OCCCCCCOc1ccc2cc(C(=O)OCCCn3cc(C)c(=O)n(CCCC)c3=O)c(=O)oc2c1]

Clause 11. A composition made by a free radical polymerization of a reactive monomer mixture comprising a compound having the chemical structure depicted by Formula I:

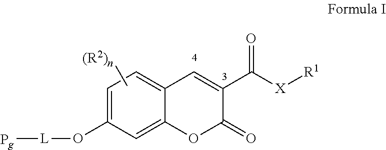

wherein P_{g} is a polymerizable group, L is a linking group, X is O or NH, R¹ is a substituent with at least one double bond capable of undergoing an intramolecular [2+2]cycloaddition reaction with the coumarin double bond in the 3-4 position to form a multi-ring structure including one cyclobutane ring, and R² is independently at each occurrence hydrogen, a C₁-C₆ alkyl group, a C₁-C₆ alkoxy group, a C₁-C₆ thioalkyl group, a C₃-C₇ cycloalkyl group, phenyl, benzyl, halo, hydroxy, amino, or NR³R⁴, wherein R³ and R⁴ are independently hydrogen or a C₁-C₆ alkyl group.

Clause 12. The composition of clause 11 wherein the polymerizable group is (meth)acrylate, (meth)acrylamide, N-vinyl lactam, N-vinyl amide, vinyl carbonate, vinyl carbamate, vinyl ether, or styryl.

Clause 13. The composition of clause 12 wherein the polymerizable group is (meth)acrylate.

Clause 14. The composition of any one of clauses 11 to 13 wherein the reactive monomer mixture contains between 15 weight percent and 80 weight percent of the compound depicted by Formula I.

Clause 15. The composition of clause 14 wherein the reactive monomer mixture contains between 20 weight percent and 60 weight percent of the compound depicted by Formula I.

Clause 16. The composition of clause 15 wherein the reactive monomer mixture contains between 20 weight percent and 50 weight percent of the compound depicted by Formula I.

Clause 17. The composition of any one of clauses 11 to 16 wherein the linking group comprises an alkylene, oxaalkylene, alkyleneoxy, cycloalkylene, heterocycloalkylene, arylene, heteroarylene, alkylene-amide-alkylene, alkylene-amine-alkylene, or combinations thereof.

Clause 18. The composition of clause 17 wherein the linking group is an alkylene having between one and twenty-five carbon atoms.

Clause 19. The composition of clause 18 wherein the alkylene is hexamethylene (CH₂)₆.

Clause 20. The composition of any one of clauses 11 to 19 wherein the R¹ of the compound depicted by Formula I has the chemical structure:

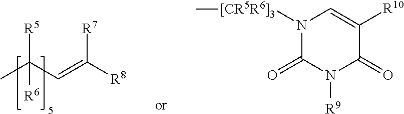

wherein R⁵, R⁶, R⁷, and R⁸ are independently hydrogen or a C₁-C₆ alkyl group, or when R⁷ and R⁸ are connected to form cycloaliphatic ring, the cycloaliphatic ring can contain between five and eight carbon atoms; and wherein R⁹ and R¹⁰ are independently hydrogen or a C₁-C₂₀ alkyl group.

Clause 21. The composition of clause 11 wherein the compound is 3,7-dimethyloct-6-en-1-yl 7-((6-(acryloyloxy)hexyl)oxy)-2-oxo-2H-chromene-3-carboxylate, having the chemical structure shown in Formula II:

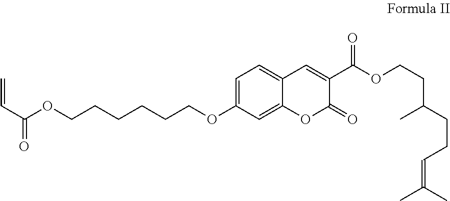 [SMILES: C=CC(=O)OCCCCCCOc1ccc2cc(C(=O)OCCC(C)CCC=C(C)C)c(=O)oc2c1]

Clause 22. The composition of clause 11 wherein the compound is 3-(3-(2-ethylhexyl)-5-methyl-2,4-dioxo-3,4-dihydropyrimidin-1(2H)-yl)propyl-7-((6-(acryloyloxy)hexyl)oxy)-2-oxo-2H-chromene-3-carboxylate, having the chemical structure shown in Formula III:

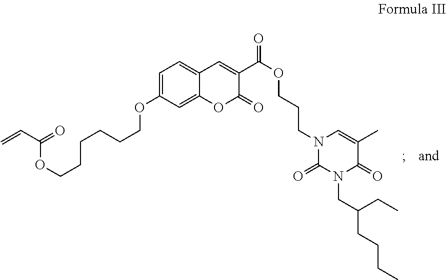 [SMILES: C=CC(=O)OCCCCCCOc1ccc2cc(C(=O)OCCCn3cc(C)c(=O)n(CC(CC)CCCC)c3=O)c(=O)oc2c1]

Clause 23. The composition of clause 11 wherein the compound is 3-(3-butyl-5-methyl-2,4-dioxo-3,4-dihydropyrimidin-1(2H)-yl)propyl-7-((6-(acryloyloxy)hexyl)oxy)-2-oxo-2H-chromene-3-carboxylate, having the chemical structure shown in Formula IV:

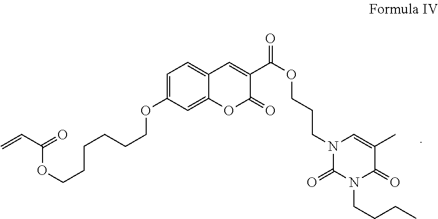 [SMILES: C=CC(=O)OCCCCCCOc1ccc2cc(C(=O)OCCCn3cc(C)c(=O)n(CCCC)c3=O)c(=O)oc2c1]

Clause 24. The composition of any one of clauses 11 to 23 further comprising an additional monomer selected from the group consisting of an aliphatic (meth)acrylate, a cycloaliphatic (meth)acrylate, an aryl (meth)acrylate, ((meth)acryloxy)alkyl alkanoates, and combinations thereof.

Clause 25. The composition of clause 24 wherein the aliphatic (meth)acrylate comprises alkyl groups having between one and twenty-five carbon atoms.

Clause 26. The composition of clause 24 wherein the cycloaliphatic (meth)acrylate comprises cycloalkyl groups having between one and twenty-five carbon atoms.

Clause 27. The composition of clause 24 wherein the ((meth)acryloxy)alkyl alkanoate comprises between one and thirty carbon atoms.

Clause 28. The composition of clause 24 wherein the additional monomer has one or more hydroxy groups.

Clause 29. The composition of any one of clauses 11 to 28 further comprising a cross-linking agent selected from the group consisting of an aliphatic cross-linking agent, a cycloaliphatic cross-linking agent, an aryl cross-linking agents, and combinations thereof.

Clause 30. The composition of clause 29 wherein the aliphatic cross-linking agent has between one and twenty-five carbon atoms.

Clause 31. The composition of clause 29 wherein the cycloaliphatic cross-linking agent has between one and twenty-five carbon atoms.

Clause 32. The composition of any one of clauses 11 to 31 further comprising a free radical initiator.

Clause 33. The composition of clause 32 wherein the free radical initiator is selected from the group consisting of a thermal initiator, a photochemical initiator, and combinations thereof.

Clause 34. The composition of any one of clauses 11 to 33 further comprising a light absorbing compound.

Clause 35. The composition of clause 34 wherein the light absorbing compound is selected from the group consisting of an ultraviolet light absorber, ultraviolet-high energy visible light absorber, visible light absorber, and combinations thereof.

Clause 36. The composition of clause 35 wherein the ultraviolet light absorber is a benzotriazole.

Clause 37. The composition of clause 36 wherein the benzotriazole is selected from the group consisting of 2-(2′-hydroxy-5-methacrylyloxyethylphenyl)-2H-benzotriazole, 3-(3-(tert-butyl)-5-(5-chloro-2H-benzo[d][1,2,3]triazol-2-yl)-4-hydroxyphenyl)-propyl methacrylate, and 2-(tert-butyl)-6-(5-chloro-2H-benzo[d][1,2,3]triazol-2-yl)-4-vinylphenol.

Clause 38. The composition of clause 35 wherein the ultraviolet-high energy visible light absorber has the chemical structure depicted by Formula V:

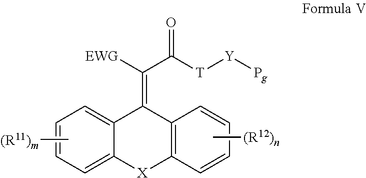

wherein m and n are independently 0, 1, 2, 3, or 4; T is a bond, O, or NR¹³; X is O, S, NR, SO, or SO₂; Y is a linking group; P_{g} is a polymerizable group; R¹³ at each occurrence is independently H, C₁-C₆ alkyl, cycloalkyl, heterocycloalkyl, aryl, heteroaryl, or Y—P_{g}; R″ and R¹², when present, are independently at each occurrence C₁-C₆ alkyl, C₁-C₆ alkoxy, C₁-C₆ thioalkyl, C₃-C₇ cycloalkyl, aryl (preferably unsubstituted phenyl or phenyl substituted with alkyl or halo), halo, hydroxy, amino, NR¹⁴R¹⁵, or benzyl, wherein R¹⁴ and R¹⁵ are independently H or C₁-C₆ alkyl, or two adjacent R¹¹ or R¹² groups, together with the carbon atoms to which they are attached, combine to form a cycloalkyl or aryl ring; and EWG is an electron withdrawing group.

Clause 39. The composition of clause 38 wherein the ultraviolet-high energy visible light absorber has the chemical structure depicted by Formula V and the EWG is cyano.

Clause 40. The composition of any one of clauses 38 to 39 wherein the ultraviolet-high energy visible light absorber is selected from the group consisting of 2-(2-cyano-2-(9H-thioxanthen-9-ylidene)acetamido)ethyl methacrylate, 2-(2-cyano-2-(9H-xanthen-9-ylidene)acetamido)ethyl methacrylate, 2-(2-cyano-2-(10-methylacridin-9(10H)-ylidene)acetamido)ethyl methacrylate, 2-(2-cyano-2-(2-methoxy-10-propylacridin-9(10H)-ylidene)acetamido)ethyl methacrylate, 2-(2-cyano-2-(2-methoxy-10-butylacridin-9(10H)-ylidene)acetamido)ethyl methacrylate, 2-(2-cyano-2-(9H-xanthen-9-ylidene)acetamido)ethyl methacrylate, (E)-2-(2-cyano-2-(3-methoxy-9H-xanthen-9-ylidene)acetamido)ethyl methacrylate, (E)-2-(2-(3-butoxy-9H-xanthen-9-ylidene)-2-cyanoacetamido)ethyl methacrylate, and any combination thereof.

Clause 41. The composition of clause 35 wherein the ultraviolet-high energy visible light absorber has the chemical structure depicted by Formula VI:

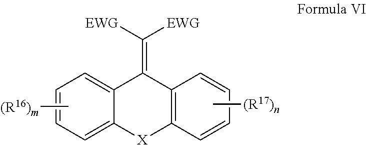

wherein m and n are independently 0, 1, 2, 3, or 4; X is O, S, NR¹⁸, SO, or SO₂; R¹⁸ at each occurrence is independently H, C₁-C₆ alkyl, cycloalkyl, heterocycloalkyl, aryl, heteroaryl, or Y—P_{g}, wherein Y is a linking group and P_{g} is a polymerizable group; R¹⁶ and R¹⁷, when present, are independently at each occurrence C₁-C₆ alkyl, C₁-C₆ alkoxy, C₁-C₆ thioalkyl, C₃-C₇ cycloalkyl, aryl (preferably unsubstituted phenyl or phenyl substituted with alkyl or halo), halo, hydroxy, amino, NR¹⁹R²⁰, or benzyl, wherein R¹⁹ and R²⁰ are independently H or C₁-C₆ alkyl, two adjacent R¹⁶ or R¹⁷ groups, together with the carbon atoms to which they are attached, combine to form a cycloalkyl or aryl ring, Y—P_{g}, or T-Y—P_{g}, wherein T is a bond, O, or NR¹⁸; and EWG is an electron withdrawing group.

Clause 42. The composition of clause 41 wherein the ultraviolet-high energy visible light absorber has the chemical structure depicted by Formula VI and the EWG is cyano.

Clause 43. The composition of any one of clauses 41 to 42 wherein the ultraviolet-high energy visible light absorber is 3-((9-(dicyanomethylene)-9H-xanthen-3-yl)oxy)propyl methacrylate.

Clause 44. The composition of any of clauses 11 to 43 wherein the water content is less than or equal to 15 weight percent.

Clause 45. The composition of clause 44 wherein the water content is less than or equal to 5 weight percent.

Clause 46. The composition of any of clauses 11 to 45 wherein the unexposed Abbe number is greater than or equal to 25.

Clause 47. The composition of clause 46 wherein the unexposed Abbe number is greater than or equal to 45.

Clause 48. The composition of any of clauses 11 to 47 wherein the unexposed refractive index is greater than or equal to 1.40.

Clause 49. The composition of clause 48 wherein the unexposed refractive index is greater than or equal to 1.50.

Clause 50. An ophthalmic device made from any one of the compositions of clauses 11 to 49.

Clause 51. The ophthalmic device of clause 50 wherein the concentration of the ultraviolet light absorber controls the amount and the location of the intramolecular [2+2]cycloaddition reactions.

Clause 52. The ophthalmic device of clause 50 wherein the concentration of the ultraviolet-high energy visible light absorber controls the amount and the location of the intramolecular [2+2]cycloaddition reactions.

Clause 53. The ophthalmic device of clause 50 further comprising a coating.

Clause 54. The ophthalmic device of clause 51 wherein the coating absorbs light selected from the group consisting of ultraviolet light, HEV light, and combinations thereof.

Clause 55. The ophthalmic device of clause 54 wherein the coating controls the amount and the location of the intramolecular [2+2]cycloaddition reactions.

Clause 56. An ophthalmic device made by a free radical polymerization of a reactive monomer mixture comprising:
(a) a compound having the chemical structure depicted in Formula I:

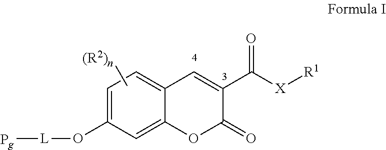

wherein P_{g} is a polymerizable group, L is a linking group, X is O or NH, R¹ is a substituent with at least one double bond capable of undergoing an intramolecular [2+2]cycloaddition reaction with the coumarin double bond in the 3-4 position to form a multi-ring structure including one cyclobutane ring, and R² is independently at each occurrence hydrogen, a C₁-C₆ alkyl group, a C₁-C₆ alkoxy group, a C₁-C₆ thioalkyl group, a C₃-C₇ cycloalkyl group, phenyl, benzyl, halo, hydroxy, amino, or NR³R⁴, wherein R³ and R⁴ are independently hydrogen or a C₁-C₆ alkyl group;
(b) one or more monomers suitable for making the ophthalmic device;
(c) a cross-linking agent; and
(d) a free radical initiator.

Clause 57. The ophthalmic device of clause 56 wherein the polymerizable group comprises (meth)acrylate, (meth)acrylamide, N-vinyl lactam, N-vinyl amide, vinyl carbonate, vinyl carbamate, vinyl ether, or styryl.

Clause 58. The ophthalmic device of clause 57 wherein the polymerizable group is (meth)acrylate.

Clause 59. The ophthalmic device of any one of clauses 56 to 58 wherein the reactive monomer mixture contains between 15 weight percent and 80 weight percent of the compound depicted by Formula I.

Clause 60. The ophthalmic device of clause 59 wherein the reactive monomer mixture contains between 20 weight percent and 60 weight percent of the compound depicted by Formula I.

Clause 61. The ophthalmic device of clause 60 wherein the reactive monomer mixture contains between 20 weight percent and 50 weight percent of the compound depicted by Formula I.

Clause 62. The ophthalmic device of any one of clauses 56 to 61 wherein the linking group comprises an alkylene, oxaalkylene, alkyleneoxy, cycloalkylene, heterocycloalkylene, arylene, heteroarylene, alkylene-amide-alkylene, alkylene-amine-alkylene, or combinations thereof.

Clause 63. The ophthalmic device of clause 62 wherein the linking group is an alkylene having between one and twenty-five carbon atoms.

Clause 64. The ophthalmic device of clause 63 wherein the alkylene is hexamethylene (CH₂)₆.

Clause 65. The ophthalmic device of any one of clauses 56 to 64 wherein the R¹ of the compound depicted by Formula I has the chemical structure:

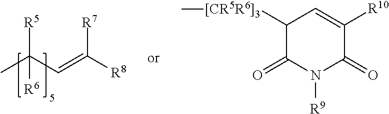

wherein R⁵, R⁶, R⁷, and R⁸ are independently hydrogen or a C₁-C₆ alkyl group, or when R⁷ and R⁸ are connected to form cycloaliphatic ring, the cycloaliphatic ring can contain between five and eight carbon atoms; and wherein R⁹ and R¹⁰ are independently hydrogen or a C₁-C₂₀ alkyl group.

Clause 66. The ophthalmic device of clause 56 wherein the compound is 3,7-dimethyloct-6-en-1-yl 7-((6-(acryloyloxy)hexyl)oxy)-2-oxo-2H-chromene-3-carboxylate, having the chemical structure shown in Formula II:

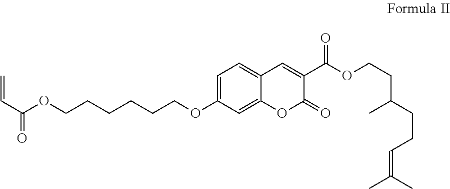 [SMILES: C=CC(=O)OCCCCCCOc1ccc2cc(C(=O)OCCC(C)CCC=C(C)C)c(=O)oc2c1]

Clause 67. The ophthalmic device of clause 56 wherein the compound is 3-(3-(2-ethylhexyl)-5-methyl-2,4-dioxo-3,4-dihydropyrimidin-1(2H)-yl)propyl 7-((6-(acryloyloxy)hexyl)oxy)-2-oxo-2H-chromene-3-carboxylate, having the chemical structure shown in Formula III:

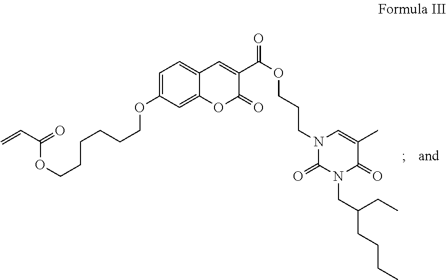 [SMILES: C=CC(=O)OCCCCCCOc1ccc2cc(C(=O)OCCCn3cc(C)c(=O)n(CC(CC)CCCC)c3=O)c(=O)oc2c1]

Clause 68. The ophthalmic device of clause 56 wherein the compound is 3-(3-butyl-5-methyl-2,4-dioxo-3,4-dihydropyrimidin-1(2H)-yl)propyl-7-((6-(acryloyloxy)hexyl)oxy)-2-oxo-2H-chromene-3-carboxylate, having the chemical structure shown in Formula IV:

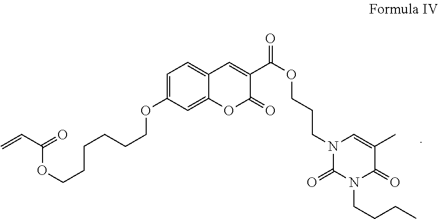 [SMILES: C=CC(=O)OCCCCCCOc1ccc2cc(C(=O)OCCCn3cc(C)c(=O)n(CCCC)c3=O)c(=O)oc2c1]

Clause 69. The ophthalmic device of any one of clauses 56 to 68 wherein the monomer suitable for making the ophthalmic device is selected from the group consisting of the aliphatic (meth)acrylate, a cycloaliphatic (meth)acrylate, an aryl (meth)acrylate, ((meth)acryloxy)alkyl alkanoates, and combinations thereof.

Clause 70. The ophthalmic device of clause 69 wherein the aliphatic (meth)acrylate comprises alkyl groups having between one and twenty-five carbon atoms.

Clause 71. The ophthalmic device of clause 69 wherein the cycloaliphatic (meth)acrylate comprises cycloalkyl groups having between one and twenty-five carbon atoms.

Clause 72. The ophthalmic device of clause 69 wherein the ((meth)acryloxy)alkyl alkanoate comprises have between one and thirty carbon atoms.

Clause 73. The ophthalmic device of clause 69 wherein the monomer has one or more hydroxy groups.

Clause 74. The ophthalmic device of any one of clauses 56 to 73 wherein the cross-linking agent selected from the group consisting of an aliphatic cross-linking agent, a cycloaliphatic cross-linking agent, an aryl cross-linking agents, and combinations thereof.

Clause 75. The ophthalmic device of clause 74 wherein the aliphatic cross-linking agent has between one and twenty-five carbon atoms.

Clause 76. The ophthalmic device of clause 74 wherein the cycloaliphatic cross-linking agent has between one and twenty-five carbon atoms.

Clause 77. The ophthalmic device of any one of clauses 56 to 76 wherein the free radical initiator is selected from the group consisting of a thermal initiator, a photochemical initiator, and combinations thereof.

Clause 78. The ophthalmic device of any one of clauses 56 to 77 further comprising a light absorbing compound.

Clause 79. The ophthalmic device of clause 78 wherein the light absorbing compound is selected from the group consisting of an ultraviolet light absorber, ultraviolet-high energy visible light absorber, visible light absorber, and combinations thereof.

Clause 80. The ophthalmic device of clause 79 wherein the ultraviolet light absorber is a benzotriazole.

Clause 81. The ophthalmic device of clause 80 wherein the benzotriazole is selected from the group consisting of 2-(2′-hydroxy-5-methacrylyloxyethylphenyl)-2H-benzotriazole, 3-(3-(tert-butyl)-5-(5-chloro-2H-benzo[d][1,2,3]triazol-2-yl)-4-hydroxyphenyl)-propyl methacrylate, and 2-(tert-butyl)-6-(5-chloro-2H-benzo[d][1,2,3]triazol-2-yl)-4-vinylphenol.

Clause 82. The ophthalmic device of clause 79 wherein the ultraviolet-high energy visible light absorber has the chemical structure depicted by Formula V:

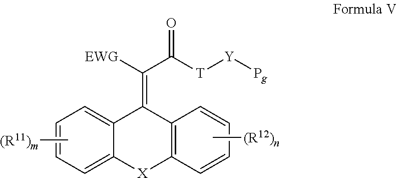

wherein m and n are independently 0, 1, 2, 3, or 4; T is a bond, O, or NR¹³; X is O, S, NR, SO, or SO₂; Y is a linking group; P_{g} is a polymerizable group; R¹³ at each occurrence is independently H, C₁-C₆ alkyl, cycloalkyl, heterocycloalkyl, aryl, heteroaryl, or Y—P_{g}; R″ and R¹², when present, are independently at each occurrence C₁-C₆ alkyl, C₁-C₆ alkoxy, C₁-C₆ thioalkyl, C₃-C₇ cycloalkyl, aryl (preferably unsubstituted phenyl or phenyl substituted with alkyl or halo), halo, hydroxy, amino, NR¹⁴R¹⁵, or benzyl, wherein R¹⁴ and R¹⁵ are independently H or C₁-C₆ alkyl, or two adjacent R¹¹ or R¹² groups, together with the carbon atoms to which they are attached, combine to form a cycloalkyl or aryl ring; and EWG is an electron withdrawing group.

Clause 83. The ophthalmic device of clause 82 wherein the ultraviolet-high energy visible light absorber has the chemical structure depicted by Formula V and the EWG is cyano.

Clause 84. The ophthalmic device of any one of clauses 82 to 83 wherein the ultraviolet-high energy visible light absorber is selected from the group consisting of 2-(2-cyano-2-(9H-thioxanthen-9-ylidene)acetamido)ethyl methacrylate, 2-(2-cyano-2-(9H-xanthen-9-ylidene)acetamido)ethyl methacrylate, 2-(2-cyano-2-(10-methylacridin-9(10H)-ylidene)acetamido)ethyl methacrylate, 2-(2-cyano-2-(2-methoxy-10-propylacridin-9(10H)-ylidene)acetamido)ethyl methacrylate, 2-(2-cyano-2-(2-methoxy-10-butylacridin-9(10H)-ylidene)acetamido)ethyl methacrylate, and any combination thereof.

Clause 85. The ophthalmic device of clause 79 wherein the ultraviolet-high energy visible light absorber has the chemical structure depicted by Formula VI:

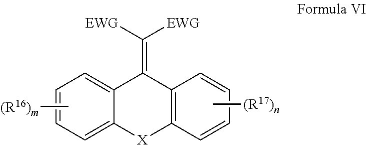

wherein m and n are independently 0, 1, 2, 3, or 4; X is O, S, NR¹⁸, SO, or SO₂; R¹⁸ at each occurrence is independently H, C₁-C₆ alkyl, cycloalkyl, heterocycloalkyl, aryl, heteroaryl, or Y—P_{g}, wherein Y is a linking group and P_{g} is a polymerizable group; R¹⁶ and R¹⁷, when present, are independently at each occurrence C₁-C₆ alkyl, C₁-C₆ alkoxy, C₁-C₆ thioalkyl, C₃-C₇ cycloalkyl, aryl (preferably unsubstituted phenyl or phenyl substituted with alkyl or halo), halo, hydroxy, amino, NR¹⁹R²⁰, or benzyl, wherein R¹⁹ and R²⁰ are independently H or C₁-C₆ alkyl, two adjacent R¹⁶ or R¹⁷ groups, together with the carbon atoms to which they are attached, combine to form a cycloalkyl or aryl ring, Y—P_{g}, or T-Y—P_{g}, wherein T is a bond, O, or NR¹⁸; and EWG is an electron withdrawing group.

Clause 86. The ophthalmic device of clause 85 wherein the ultraviolet-high energy visible light absorber has the chemical structure depicted by Formula VI and the EWG is cyano.

Clause 87. The ophthalmic device of any one of clauses 85 to 86 wherein the ultraviolet-high energy visible light absorber is 3-((9-(dicyanomethylene)-9H-xanthen-3-yl)oxy)propyl methacrylate.

Clause 88. The ophthalmic device of any of clauses 56 to 87 wherein the water content is less than or equal to 15 weight percent.

Clause 89. The ophthalmic device of clause 88 wherein the water content is less than or equal to 5 weight percent.

Clause 90. The ophthalmic device of any of clauses 56 to 89 wherein the unexposed Abbe number is greater than or equal to 25.

Clause 91. The ophthalmic device of clause 90 wherein the unexposed Abbe number is greater than or equal to 45.

Clause 92. The ophthalmic device of any of clauses 56 to 91 wherein the unexposed refractive index is greater than or equal to 1.40.

Clause 93. The ophthalmic device of clause 92 wherein the unexposed refractive index is greater than or equal to 1.50.

Clause 94. The ophthalmic device of any one of clauses 56 to 93 wherein the concentration of the ultraviolet light absorber controls the amount and the location of the intramolecular [2+2]cycloaddition reactions.

Clause 95. The ophthalmic device of any one of clauses 56 to 93 wherein the concentration of the ultraviolet-high energy visible light absorber controls the amount and the location of the intramolecular [2+2]cycloaddition reactions.

Clause 96. The ophthalmic device of any one of clauses 56 to 95 further comprising a coating.

Clause 97. The ophthalmic device of clause 96 wherein the coating absorbs ultraviolet light, high energy visible light, or combinations thereof.

Clause 98. The ophthalmic device of clause 97 wherein the coating absorbs ultraviolet light and controls the amount and the location of the intramolecular [2+2]cycloaddition reactions.

Clause 99. The ophthalmic device of any one of clauses 56 to 98 wherein the refractive index is changed at least in selected regions by irradiation of the ophthalmic device with ultraviolet light having a wavelength shorter than or equal to 400 nanometers during which the intramolecular [2+2]cycloaddition reaction occurs by a single photon mechanism.

Clause 100. The ophthalmic device of any one of clauses 56 to 98 wherein the refractive index is changed at least in selected regions by irradiation of the ophthalmic device with visible light having a wavelength longer than or equal to 650 nanometers during which the intramolecular [2+2]cycloaddition reaction occurs by a two-photon or multi-photon absorption mechanism.

Clause 101. The ophthalmic device of any one of clauses 56 to 100 wherein the ophthalmic device is selected from the group consisting of an intraocular lens, phakic intraocular lens, contact lens, corneal inlay, corneal outlay, or corneal insert.

Clause 102. The ophthalmic device of clause 101 wherein the ophthalmic device is an intraocular lens.

Clause 103. A method for making an ophthalmic device, the method comprising:
(a) providing a composition of any one of clauses 11 to 49; and
(b) forming an ophthalmic device.

Clause 104. A method for making an ophthalmic device, the method comprising:
(a) preparing a blank from the composition any of clauses to 11 to 49; and
(b) machining an ophthalmic device from the blank.

Clause 105. A method for making an ophthalmic device, the method comprising:
(a) molding the device from the composition of any of clauses 11 to 49.

Clause 106. A method for making an ophthalmic device, the method comprising:
(a) providing a reactive monomer mixture, comprising a compound having a chemical structure selected from the group consisting of Formula I, Formula II, Formula III, Formula IV, or combinations thereof, in a mold assembly;
(b) forming an ophthalmic device by a free radical polymerization; and
(c) demolding the ophthalmic device from the mold assembly.

Clause 107. A method for making an ophthalmic device, the method comprising:
(a) providing a reactive monomer mixture, comprising a compound having a chemical structure selected from the group consisting of Formula I, Formula II, Formula III, Formula IV, or combinations thereof, in a mold assembly;
(b) forming an ophthalmic device by a free radical photopolymerization reaction; and
(c) demolding the ophthalmic device from the mold assembly.

Clause 108. The method of clause 107, wherein the free radical photopolymerization reaction comprises irradiating the mold assembly from the top and the bottom with 435 nanometer light emitting diodes having an intensity profile:
(a) 2250 seconds at 5 mW/cm² (2.5 mW/cm² top and 2.5 mW/cm² bottom)
(b) 2250 seconds at 10 mW/cm² (5 mW/cm² top and 5 mW/cm² bottom); and
(c) 4500 seconds at 20 mW/cm² (10 mW/cm² top and 10 mW/cm² bottom).

Clause 109. A method for making an ophthalmic device, the method comprising:
(a) providing a reactive monomer mixture, comprising a compound having a chemical structure selected from the group consisting of Formula I, Formula II, Formula III, Formula IV, or combinations thereof, in a mold assembly;
(b) forming an ophthalmic device by a free radical thermal polymerization reaction; and
(c) demolding the ophthalmic device from the mold assembly.

Clause 110. The method of clause 109 wherein the free radical thermal polymerization is conducted at temperatures between 40° C. and 120° C.; between 40° C. and 100° C.; or between 40° C. and 90° C.

Clause 111. The method of clause 110 wherein the free radical thermal polymerization is conducted in stages at different temperatures.

Clause 112. The method of any of clauses 103 to 111, further comprising the step of extracting the ophthalmic device with a solvent.

Clause 113. The method of clause 112 wherein the solvent is selected from the group consisting of acetonitrile, isopropanol, ethyl acetate, ethanol, and mixtures thereof, and aqueous solutions of acetonitrile or isopropanol or ethanol.

Clause 114. The method of any of clauses 103 to 111, further comprising the step of annealing the ophthalmic device with a vacuum oven, heated nitrogen environment, heated dry air environment, or any combination thereof.

Clause 115. The method of any of clauses 103 to 114, further comprising the step of hydrating the extracted ophthalmic device with at least one aqueous solution.

Clause 116. The method of any of clauses 103 to 115, further comprising a step of sterilizing the ophthalmic device.

Clause 117. The method of clause 116, further comprising a step of irradiating the ophthalmic device using a femtosecond laser or diode laser either before or after sterilization.

Clause 118. The method of clause 117, comprising a step of irradiating the ophthalmic device using a with ultraviolet light having a wavelength shorter than or equal to 400 nanometers during which an intramolecular [2+2]cycloaddition reaction occurs by a single photon mechanism.

Clause 119. The method of clause 117, comprising a step of irradiating the ophthalmic device with visible light having a wavelength longer than or equal to 650 nanometers during which the intramolecular [2+2]cycloaddition reaction occurs by a two-photon or multi-photon absorption mechanism.

Clause 120. The method of any one of clauses 117 to 119, wherein the irradiation step is performed on an implanted ophthalmic device.

Clause 121. The method of any one of clauses 113 to 120 wherein the irradiation is conducted in selected regions of the ophthalmic device.

Clause 122. The method of any of clauses 103 to 121, wherein the ophthalmic device is selected from the group consisting of an intraocular lens, phakic intraocular lens, contact lens, corneal inlay, corneal outlay, or corneal insert.

Clause 123. The method of clause 122, wherein the ophthalmic device is an intraocular lens.

Clause 124. An ophthalmic device made by a free radical polymerization of a reactive monomer mixture comprising:
a) 3,7-dimethyloct-6-en-1-yl 7-((6-(acryloyloxy)hexyl)oxy)-2-oxo-2H-chromene-3-carboxylate, having the chemical structure shown in Formula II:

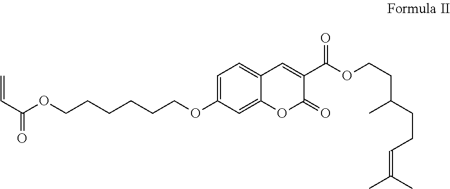 [SMILES: C=CC(=O)OCCCCCCOc1ccc2cc(C(=O)OCCC(C)CCC=C(C)C)c(=O)oc2c1]

b) 4-hydroxybutyl acrylate;
c) N-butyl acrylate;
d) ethyl methacrylate;
e) ethylene glycol dimethacrylate; and
f) azobisisobutyronitrile;
   wherein the concentration of Formula II in the reactive monomer mixture is between 20 and 35 weight percent; wherein the concentration of 4-hydroxybutyl acrylate in the reactive monomer mixture is between 15 and 20 weight percent; wherein the concentration of N-butyl acrylate in the reactive monomer mixture is between 5 and 20 weight percent; wherein the concentration of ethyl methacrylate in the reactive monomer mixture is between 25 and 45 weight percent; wherein the concentration of ethylene glycol dimethacrylate in the reactive monomer mixture is between 1 and 3 weight percent; and wherein the concentration of azobisisobutyronitrile in the reactive monomer mixture is between 0.1 and 3 weight percent.

Clause 125. The ophthalmic device of clause 124 further comprising 3-((9-(dicyanomethylene)-9H-xanthen-3-yl)oxy)propyl methacrylate wherein the concentration of 3-((9-(dicyanomethylene)-9H-xanthen-3-yl)oxy)propyl methacrylate in the reactive monomer mixture is between 0.1 and 2 weight percent.

Clause 126. The ophthalmic device of clause 125 further comprising 2-(tert-butyl)-6-(5-chloro-2H-benzo[d][1,2,3]triazol-2-yl)-4-vinylphenol wherein the concentration of 2-(tert-butyl)-6-(5-chloro-2H-benzo[d][1,2,3]triazol-2-yl)-4-vinylphenol in the reactive monomer mixture is between 0.1 and 2 weight percent.

Clause 127. The ophthalmic device of any one of clauses 124 to 126 wherein the ophthalmic device is an intraocular lens.

Clause 128. An ophthalmic device made by a free radical polymerization of a reactive monomer mixture comprising:
a) a compound comprising a polymerizable group and at least two double bonds capable of undergoing a [2+2]cycloaddition reaction;
b) one or more monomers suitable for making the ophthalmic device;
c) a cross-linking agent;
d) a free radical initiator; and
e) one or more light absorbers selected from ultraviolet (UV) and ultraviolet/high energy visible (HEV) light absorbers, present in a total amount effective to form a protective layer that limits transmission of UV and/or HEV light beyond a penetration depth from a surface of the ophthalmic device.

Clause 120. The ophthalmic device of clause 128, wherein the protective layer has a thickness that does not exceed about 500 microns, about 400 microns, about 300 microns, about 200 microns, about 100 microns, about 50 microns, or about 25 microns.

Clause 130. The ophthalmic device of clause 128 or clause 129, wherein the one or more light absorbers are present in the reactive monomer mixture in a total amount from about 0.2% to about 5% by weight, about 0.2% to about 1.0% by weight, about 0.5% to about 2.5% by weight, about 2.0% to about 3.0% by weight, about 3.0% to about 4.0% by weight, or about 4.0% to about 5.0% by weight.

Clause 131. The ophthalmic device of any one of clauses 128 to 130, wherein the light absorber is a benzotriazole.

Clause 132. The ophthalmic device of clause 131, wherein the benzotriazole is selected from the group consisting of 2-(2′-hydroxy-5-methacrylyloxyethylphenyl)-2H-benzotriazole, 3-(3-(tert-butyl)-5-(5-chloro-2H-benzo[d][1,2,3]triazol-2-yl)-4-hydroxyphenyl)-propyl methacrylate, and 2-(tert-butyl)-6-(5-chloro-2H-benzo[d][1,2,3]triazol-2-yl)-4-vinylphenol.

Clause 133. The ophthalmic device of any one of clauses 128 to 130, wherein the light absorber has the chemical structure depicted by Formula V:

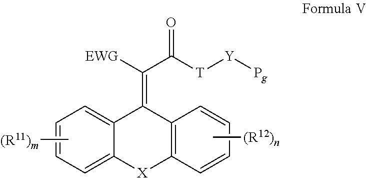

wherein m and n are independently 0, 1, 2, 3, or 4; T is a bond, O, or NR¹³; X is O, S, NR, SO, or 502; Y is a linking group; P_{g} is a polymerizable group; R¹³ at each occurrence is independently H, C₁-C₆ alkyl, cycloalkyl, heterocycloalkyl, aryl, heteroaryl, or Y—P_{g}; R″ and R¹², when present, are independently at each occurrence C₁-C₆ alkyl, C₁-C₆ alkoxy, C₁-C₆ thioalkyl, C₃-C₇ cycloalkyl, aryl (preferably unsubstituted phenyl or phenyl substituted with alkyl or halo), halo, hydroxy, amino, NR¹⁴R¹⁵, or benzyl, wherein R¹⁴ and R¹⁵ are independently H or C₁-C₆ alkyl, or two adjacent R¹¹ or R¹² groups, together with the carbon atoms to which they are attached, combine to form a cycloalkyl or aryl ring; and EWG is an electron withdrawing group.

Clause 134. The ophthalmic device of clause 133, wherein the light absorber has the chemical structure depicted by Formula V and the EWG is cyano.

Clause 135. The ophthalmic device of clause 133 or clause 134, wherein the light absorber is selected from the group consisting of 2-(2-cyano-2-(9H-thioxanthen-9-ylidene)acetamido)ethyl methacrylate, 2-(2-cyano-2-(9H-xanthen-9-ylidene)acetamido)ethyl methacrylate, 2-(2-cyano-2-(10-methylacridin-9(10H)-ylidene)acetamido)ethyl methacrylate, 2-(2-cyano-2-(2-methoxy-10-propylacridin-9(10H)-ylidene)acetamido)ethyl methacrylate, 2-(2-cyano-2-(2-methoxy-10-butylacridin-9(10H)-ylidene)acetamido)ethyl methacrylate, and any combination thereof.

Clause 136. The ophthalmic device of any one of clauses 128 to 130, wherein the light absorber has the chemical structure depicted by Formula VI:

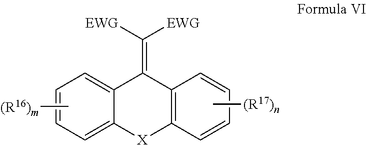

wherein m and n are independently 0, 1, 2, 3, or 4; X is O, S, NR¹⁸, SO, or SO₂; R¹⁸ at each occurrence is independently H, C₁-C₆ alkyl, cycloalkyl, heterocycloalkyl, aryl, heteroaryl, or Y—P_{g}, wherein Y is a linking group and P_{g} is a polymerizable group; R¹⁶ and R¹⁷, when present, are independently at each occurrence C₁-C₆ alkyl, C₁-C₆ alkoxy, C₁-C₆ thioalkyl, C₃-C₇ cycloalkyl, aryl (preferably unsubstituted phenyl or phenyl substituted with alkyl or halo), halo, hydroxy, amino, NR¹⁹R²⁰, or benzyl, wherein R¹⁹ and R²⁰ are independently H or C₁-C₆ alkyl, two adjacent R¹⁶ or R¹⁷ groups, together with the carbon atoms to which they are attached, combine to form a cycloalkyl or aryl ring, Y—P_{g}, or T-Y—P_{g}, wherein T is a bond, O, or NR¹⁸; and EWG is an electron withdrawing group.

Clause 137. The ophthalmic device of clause 136, wherein the light absorber has the chemical structure depicted by Formula VI and the EWG is cyano.

Clause 138. The ophthalmic device of clause 136 or clause 137, wherein the light absorber is 3-((9-(dicyanomethylene)-9H-xanthen-3-yl)oxy)propyl methacrylate.

Clause 139. The ophthalmic device of any one of clauses 128 to 138, wherein the compound comprises two double bonds capable of undergoing an intramolecular [2+2]cycloaddition to form a cyclobutane ring.

Clause 140. The ophthalmic device of any one of clauses 128 to 138, wherein the compound comprises a coumarin moiety and a substituent with at least one double bond capable of undergoing an intramolecular [2+2]cycloaddition reaction with a double bond in the 3-4 position of the coumarin to form a multi-ring structure including one cyclobutane ring.

Clause 141. The ophthalmic device of any one of clauses 128 to 138, wherein the compound has the chemical structure depicted in Formula I:

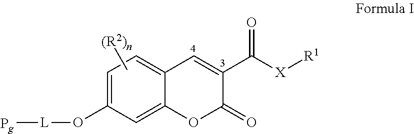

wherein P_{g} is the polymerizable group, L is a linking group, X is O or NH, R¹ is a substituent with at least one double bond capable of undergoing an intramolecular [2+2]cycloaddition reaction with the coumarin double bond in the 3-4 position to form a multi-ring structure including one cyclobutane ring, and R² is independently at each occurrence hydrogen, a C₁-C₆ alkyl group, a C₁-C₆ alkoxy group, a C₁-C₆ thioalkyl group, a C₃-C₇ cycloalkyl group, phenyl, benzyl, halo, hydroxy, amino, or NR³R⁴, wherein R³ and R⁴ are independently hydrogen or a C₁-C₆ alkyl group;

Clause 142. The ophthalmic device of clause 141, wherein the polymerizable group P_{g} comprises (meth)acrylate, (meth)acrylamide, N-vinyl lactam, N-vinyl amide, vinyl carbonate, vinyl carbamate, vinyl ether, or styryl.

Clause 143. The ophthalmic device of clause 142, wherein the polymerizable group P_{g} is (meth)acrylate.

Clause 144. The ophthalmic device of any one of clauses 141 to 143, wherein the reactive monomer mixture contains between 15 weight percent and 80 weight percent of the compound depicted by Formula I.

Clause 145. The ophthalmic device of clause 144, wherein the reactive monomer mixture contains between 20 weight percent and 60 weight percent of the compound depicted by Formula I.

Clause 146. The ophthalmic device of clause 145, wherein the reactive monomer mixture contains between 20 weight percent and 50 weight percent of the compound depicted by Formula I.

Clause 147. The ophthalmic device of any one of clauses 141 to 146, wherein the linking group comprises an alkylene, oxaalkylene, alkyleneoxy, cycloalkylene, heterocycloalkylene, arylene, heteroarylene, alkylene-amide-alkylene, alkylene-amine-alkylene, or combinations thereof.

Clause 148. The ophthalmic device of clause 147, wherein the linking group is an alkylene having between one and twenty-five carbon atoms.

Clause 149. The ophthalmic device of clause 148, wherein the alkylene is hexamethylene (CH₂)₆.

Clause 150. The ophthalmic device of any one of clauses 141 to 149, wherein the R¹ of the compound depicted by Formula I has the chemical structure:

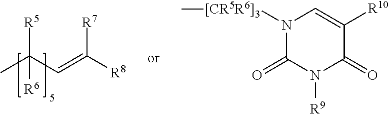

wherein R⁵, R⁶, R⁷, and R⁸ are independently hydrogen or a C₁-C₆ alkyl group, or when R⁷ and R⁸ are connected to form cycloaliphatic ring, the cycloaliphatic ring can contain between five and eight carbon atoms; and wherein R⁹ and R¹⁰ are independently hydrogen or a C₁-C₂₀ alkyl group.

Clause 151. The ophthalmic device of clause 141, wherein the compound is 3,7-dimethyloct-6-en-1-yl 7-((6-(acryloyloxy)hexyl)oxy)-2-oxo-2H-chromene-3-carboxylate, having the chemical structure shown in Formula II:

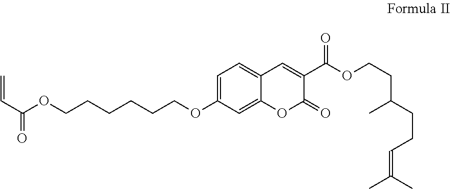 [SMILES: C=CC(=O)OCCCCCCOc1ccc2cc(C(=O)OCCC(C)CCC=C(C)C)c(=O)oc2c1]

Clause 152. The ophthalmic device of clause 141, wherein the compound is 3-(3-(2-ethylhexyl)-5-methyl-2,4-dioxo-3,4-dihydropyrimidin-1(2H)-yl)propyl-7-((6-(acryloyloxy)hexyl)oxy)-2-oxo-2H-chromene-3-carboxylate, having the chemical structure shown in Formula III:

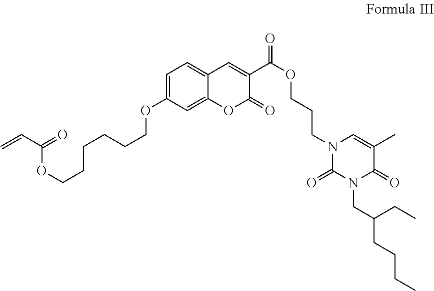 [SMILES: C=CC(=O)OCCCCCCOc1ccc2cc(C(=O)OCCCn3cc(C)c(=O)n(CC(CC)CCCC)c3=O)c(=O)oc2c1]

Clause 153. The ophthalmic device of clause 141, wherein the compound is 3-(3-butyl-5-methyl-2,4-dioxo-3,4-dihydropyrimidin-1(2H)-yl)propyl-7-((6-(acryloyloxy)hexyl)oxy)-2-oxo-2H-chromene-3-carboxylate, having the chemical structure shown in Formula IV:

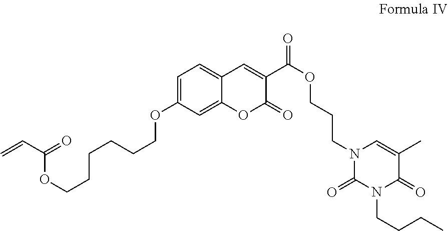 [SMILES: C=CC(=O)OCCCCCCOc1ccc2cc(C(=O)OCCCn3cc(C)c(=O)n(CCCC)c3=O)c(=O)oc2c1]

Clause 154. The ophthalmic device of any one of clauses 128 to 153, wherein the monomer suitable for making the ophthalmic device is selected from the group consisting of an aliphatic (meth)acrylate, a cycloaliphatic (meth)acrylate, an aryl (meth)acrylate, ((meth)acryloxy)alkyl alkanoates, and combinations thereof.

Clause 155. The ophthalmic device of clause 154, wherein the aliphatic (meth)acrylate comprises alkyl groups having between one and twenty-five carbon atoms.

Clause 156. The ophthalmic device of clause 154, wherein the cycloaliphatic (meth)acrylate comprises cycloalkyl groups having between one and twenty-five carbon atoms.

Clause 157. The ophthalmic device of clause 154, wherein the ((meth)acryloxy)alkyl alkanoate comprises have between one and thirty carbon atoms.

Clause 158. The ophthalmic device of clause 154, wherein the monomer has one or more hydroxy groups.

Clause 159. The ophthalmic device of any one of clauses 128 to 158, wherein the cross-linking agent selected from the group consisting of an aliphatic cross-linking agent, a cycloaliphatic cross-linking agent, an aryl cross-linking agents, and combinations thereof.

Clause 160. The ophthalmic device of clause 159, wherein the aliphatic cross-linking agent has between one and twenty-five carbon atoms.

Clause 161. The ophthalmic device of clause 159, wherein the cycloaliphatic cross-linking agent has between one and twenty-five carbon atoms.

Clause 162. The ophthalmic device of any one of clauses 128 to 161, wherein the free radical initiator is selected from the group consisting of a thermal initiator, a photochemical initiator, and combinations thereof.

Clause 163. The ophthalmic device of any of clauses 128 to 162, wherein the water content is less than or equal to 15 weight percent.

Clause 164. The ophthalmic device of clause 163, wherein the water content is less than or equal to 5 weight percent.

Clause 165. The ophthalmic device of any of clauses 128 to 164, wherein the unexposed Abbe number is greater than or equal to 25.

Clause 166. The ophthalmic device of clause 165, wherein the unexposed Abbe number is greater than or equal to 45.

Clause 167. The ophthalmic device of any of clauses 128 to 166, wherein the unexposed refractive index is greater than or equal to 1.40.

Clause 168. The ophthalmic device of clause 167, wherein the unexposed refractive index is greater than or equal to 1.50.

Clause 169. The ophthalmic device of any one of clauses 128 to 168, further comprising a coating.

Clause 170. The ophthalmic device of clause 169, wherein the coating absorbs ultraviolet light, high energy visible light, or combinations thereof.

Clause 171. The ophthalmic device of clause 170, wherein the coating absorbs ultraviolet light and controls the amount and the location of the intramolecular [2+2]cycloaddition reactions.

Clause 172. The ophthalmic device of any one of clauses 128 to 171, wherein the refractive index is changed at least in selected regions by irradiation of the ophthalmic device with ultraviolet light having a wavelength shorter than or equal to 400 nanometers during which the intramolecular [2+2]cycloaddition reaction occurs by a single photon mechanism.

Clause 173. The ophthalmic device of any one of clauses 128 to 171, wherein the refractive index is changed at least in selected regions by irradiation of the ophthalmic device with visible light having a wavelength longer than or equal to 650 nanometers during which the intramolecular [2+2]cycloaddition reaction occurs by a two-photon or multi-photon absorption mechanism.

Clause 174. The ophthalmic device of any one of clauses 128 to 173, wherein the ophthalmic device is selected from the group consisting of an intraocular lens, phakic intraocular lens, contact lens, corneal inlay, corneal outlay, or corneal insert.

Clause 175. The ophthalmic device of clause 174, wherein the ophthalmic device is an intraocular lens.

1. A compound having a chemical structure depicted by Formula I: 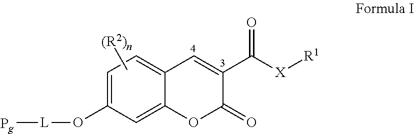
wherein P_{g} is a polymerizable group, L is a linking group, X is O or NH, R¹ is a substituent with at least one double bond capable of undergoing an intramolecular [2+2]cycloaddition reaction with the coumarin double bond in the 3-4 position to form a multi-ring structure including one cyclobutane ring, and R² is independently at each occurrence hydrogen, a C₁-C₆ alkyl group, a C₁-C₆ alkoxy group, a C₁-C₆ thioalkyl group, a C₃-C₇ cycloalkyl group, phenyl, benzyl, halo, hydroxy, amino, or NR³R⁴, wherein R³ and R⁴ are independently hydrogen or a C₁-C₆ alkyl group.

2. (canceled)

3. The compound of claim 1 wherein the polymerizable group is (meth)acrylate.

4. (canceled)

5. The compound of claim 1 wherein the linking group is an alkylene having between one and twenty-five carbon atoms.

6. (canceled)

7. The compound of claim 1 wherein the R¹ of the compound depicted by Formula I has the chemical structure: 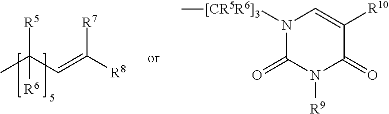
wherein R⁵, R⁶, R⁷, and R⁸ are independently hydrogen or a C₁-C₆ alkyl group, or when R⁷ and R⁸ are connected to form cycloaliphatic ring, the cycloaliphatic ring can contain between five and eight carbon atoms; and wherein R⁹ and R¹⁰ are independently hydrogen or a C₁-C₂₀ alkyl group.

8. The compound of claim 1 wherein the compound is selected from the group consisting of:
3,7-dimethyloct-6-en-1-yl 7-((6-(acryloyloxy)hexyl)oxy)-2-oxo-2H-chromene-3-carboxylate, having the chemical structure shown in Formula II: 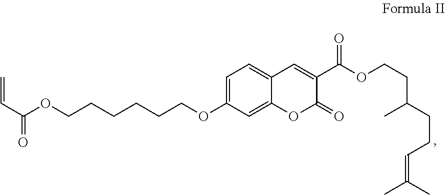 [SMILES: C=CC(=O)OCCCCCCOc1ccc2cc(C(=O)OCCC(C)CCC=C(C)C)c(=O)oc2c1]
3-(3-(2-ethylhexyl)-5-methyl-2,4-dioxo-3,4-dihydropyrimidin-1(2H)-yl)propyl-7-((6-(acryloyloxy)hexyl)oxy)-2-oxo-2H-chromene-3-carboxylate, having the chemical structure shown in Formula III: 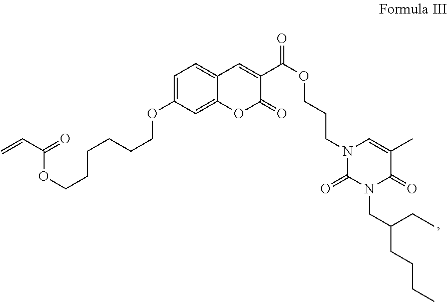 [SMILES: C=CC(=O)OCCCCCCOc1ccc2cc(C(=O)OCCCn3cc(C)c(=O)n(CC(CC)CCCC)c3=O)c(=O)oc2c1]
and
3-(3-butyl-5-methyl-2,4-dioxo-3,4-dihydropyrimidin-1(2H)-yl)propyl-7-((6-(acryloyloxy)hexyl)oxy)-2-oxo-2H-chromene-3-carboxylate, having the chemical structure shown in Formula IV:

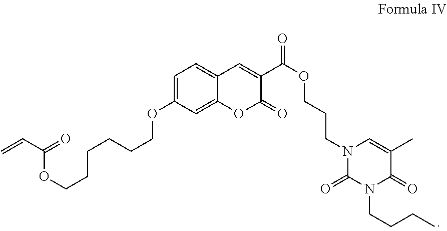 [SMILES: C=CC(=O)OCCCCCCOc1ccc2cc(C(=O)OCCCn3cc(C)c(=O)n(CCCC)c3=O)c(=O)oc2c1]

9.-10. (canceled)

11. A composition made by a free radical polymerization of a reactive monomer mixture comprising a compound of claim 1.

12.-15. (canceled)

16. The composition of claim 11 wherein the reactive monomer mixture contains between 20 weight percent and 50 weight percent of the compound depicted by Formula I.

17.-23. (canceled)

24. The composition of claim 11 further comprising at least one of:
an additional monomer selected from the group consisting of an aliphatic (meth)acrylate, a cycloaliphatic (meth)acrylate, an aryl (meth)acrylate, ((meth)acryloxy)alkyl alkanoates, and combinations thereof;
a cross-linking agent selected from the group consisting of an aliphatic cross-linking agent, a cycloaliphatic cross-linking agent, an aryl cross-linking agents, and combinations thereof;
a free radical initiator; and
a light absorbing compound.

25.-44. (canceled)

45. The composition of claim 11 wherein the water content is less than or equal to 5 weight percent, the unexposed Abbe number is greater than or equal to 45, and/or the unexposed refractive index is greater than or equal to 1.50.

46.-49. (canceled)

50. An ophthalmic device made from the composition of claim 11.

51.-55. (canceled)

56. An ophthalmic device made by a free radical polymerization of a reactive monomer mixture comprising:
a) a compound having the chemical structure depicted in Formula I: 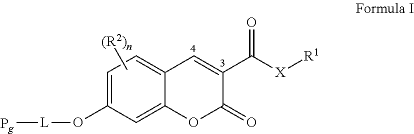
wherein P_{g} is a polymerizable group, L is a linking group, X is O or NH, R¹ is a substituent with at least one double bond capable of undergoing an intramolecular [2+2]cycloaddition reaction with the coumarin double bond in the 3-4 position to form a multi-ring structure including one cyclobutane ring, and R² is independently at each occurrence hydrogen, a C₁-C₆ alkyl group, a C₁-C₆ alkoxy group, a C₁-C₆ thioalkyl group, a C₃-C₇ cycloalkyl group, phenyl, benzyl, halo, hydroxy, amino, or NR³R⁴, wherein R³ and R⁴ are independently hydrogen or a C₁-C₆ alkyl group;
b) one or more monomers suitable for making the ophthalmic device;
c) a cross-linking agent; and
d) a free radical initiator.

57.-65. (canceled)

66. The ophthalmic device of claim 56 wherein the compound is selected from the group consisting of:
3,7-dimethyloct-6-en-1-yl 7-((6-(acryloyloxy)hexyl)oxy)-2-oxo-2H-chromene-3-carboxylate, having the chemical structure shown in Formula II: 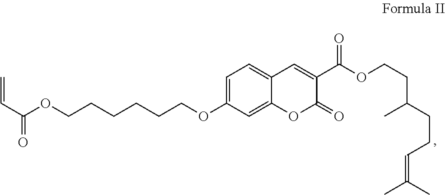 [SMILES: C=CC(=O)OCCCCCCOc1ccc2cc(C(=O)OCCC(C)CCC=C(C)C)c(=O)oc2c1]
3-(3-(2-ethylhexyl)-5-methyl-2,4-dioxo-3,4-dihydropyrimidin-1(2H)-yl)propyl-7-((6-(acryloyloxy)hexyl)oxy)-2-oxo-2H-chromene-3-carboxylate, having the chemical structure shown in Formula III: 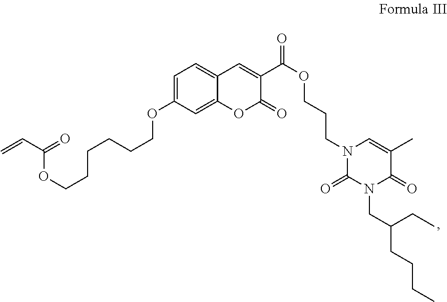 [SMILES: C=CC(=O)OCCCCCCOc1ccc2cc(C(=O)OCCCn3cc(C)c(=O)n(CC(CC)CCCC)c3=O)c(=O)oc2c1]
and
3-(3-butyl-5-methyl-2,4-dioxo-3,4-dihydropyrimidin-1(2H)-yl)propyl-7-((6-(acryloyloxy)hexyl)oxy)-2-oxo-2H-chromene-3-carboxylate, having the chemical structure shown in Formula IV:

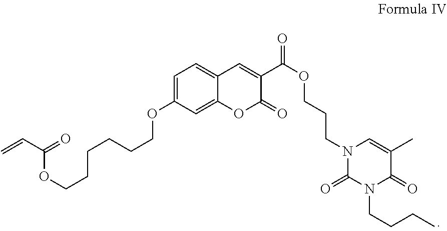 [SMILES: C=CC(=O)OCCCCCCOc1ccc2cc(C(=O)OCCCn3cc(C)c(=O)n(CCCC)c3=O)c(=O)oc2c1]

67.-100. (canceled)

101. The ophthalmic device of claim 56 wherein the ophthalmic device is selected from the group consisting of an intraocular lens, phakic intraocular lens, contact lens, corneal inlay, corneal outlay, or corneal insert.

102. (canceled)

103. A method for making an ophthalmic device, the method comprising:
a) providing a composition of claim 11; and
b) forming an ophthalmic device.

104. A method for making an ophthalmic device, the method comprising:
a) preparing a blank from the composition claim 11; and
b) machining an ophthalmic device from the blank.

105. A method for making an ophthalmic device, the method comprising:
a) molding the device from the composition claim 11.

106. A method for making an ophthalmic device, the method comprising:
a) providing a reactive monomer mixture comprising a compound of claim 1 in a mold assembly;
b) forming an ophthalmic device by a free radical polymerization; and
c) demolding the ophthalmic device from the mold assembly.

107. A method for making an ophthalmic device, the method comprising:
a) providing a reactive monomer mixture comprising a compound of claim 1 in a mold assembly;
b) forming an ophthalmic device by a free radical photopolymerization reaction; and
c) demolding the ophthalmic device from the mold assembly.

108. (canceled)

109. A method for making an ophthalmic device, the method comprising:
a) providing a reactive monomer mixture comprising a compound of claim 1 in a mold assembly;
b) forming an ophthalmic device by a free radical thermal polymerization reaction; and
c) demolding the ophthalmic device from the mold assembly.

110.-123. (canceled)

124. An ophthalmic device made by a free radical polymerization of a reactive monomer mixture comprising:
a) 3,7-dimethyloct-6-en-1-yl 7-((6-(acryloyloxy)hexyl)oxy)-2-oxo-2H-chromene-3-carboxylate, having the chemical structure shown in Formula II: 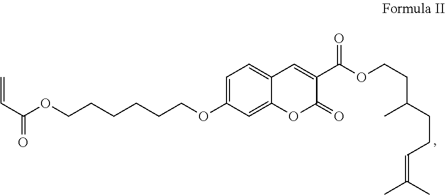 [SMILES: C=CC(=O)OCCCCCCOc1ccc2cc(C(=O)OCCC(C)CCC=C(C)C)c(=O)oc2c1]
b) 4-hydroxybutyl acrylate;
c) N-butyl acrylate;
d) ethyl methacrylate;
e) ethylene glycol dimethacrylate; and
f) azobisisobutyronitrile;
wherein the concentration of Formula II in the reactive monomer mixture is between 20 and 35 weight percent; wherein the concentration of 4-hydroxybutyl acrylate in the reactive monomer mixture is between 15 and 20 weight percent; wherein the concentration of N-butyl acrylate in the reactive monomer mixture is between 5 and 20 weight percent; wherein the concentration of ethyl methacrylate in the reactive monomer mixture is between 25 and 45 weight percent; wherein the concentration of ethylene glycol dimethacrylate in the reactive monomer mixture is between 1 and 3 weight percent; and wherein the concentration of azobisisobutyronitrile in the reactive monomer mixture is between 1 and 3 weight percent.

125.-127. (canceled)
